SEQUENCE LISTING (26 sequences)

SEQ ID NO: 1 - DNA, 1275 bp, synthetic construct
  misc_feature 1..1275: note = Synthetic: D2-1275 bp Ad2 sequence
  source 1..1275: mol_type = other DNA
ccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaactgccctcacgtccaagtggacgtaaaggctaaacccttcagggtgaatctcctctataaacattccagcaccttcaaccatgcccaaataattttcatctcgccaccttatcaatatgtctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagcccctatgtaagcttgttgcatgggcggcgatataaaatgcaaggtgctgctcaaaaaatcaggcaaagcctcgcgcaaaaaagcaagcacatcgtagtcatgctcatgcagataaaggcaggtaagttccggaaccaccacagaaaaagacaccatttttctctcaaacatgtctgcgggttcctgcattaaacacaaaataaaataacaaaaaaaaaaacatttaaacattagaagcctgtcttacaacaggaaaaacaacccttataagcataagacggactacggccatgccggcgtgaccgtaaaaaaactggtcaccgtgattaaaaagcaccaccgacagttcctcggtcatgtccggagtcataatgtaagactcggtaaacacatcaggttggttaacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcc

SEQ ID NO: 2 - DNA, 711 bp, synthetic construct
  misc_feature 1..711: note = Synthetic: D1-711 bp Ad2 sequence
  source 1..711: mol_type = other DNA
ctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcac

SEQ ID NO: 3 - DNA, 1194 bp, synthetic construct
  misc_feature 1..1194: note = Synthetic: D3-1897 bp Ad2 sequence
  source 1..1194: mol_type = other DNA
tacaaatttgcgaaggtaagccgacgtccacagccccggagtgagtttcaaccccggagccgcggacttttcgtcaggcgagggaccctgcagctcaaaggtaccgataatttgactttcgctaagcagttgcgaattgcagaccagggagcggtgcggggtgcataggttgcagcgacagtgacactccagtaggccgtcaccgctcacgtcttccatgatgtcggagtggtaggcaaggtagttggctagctgcagaaggtagcagtgaccccaaagcggcggagggcattcacggtacttaatgggcacaaagtcgctaggaagcgcacagcaggtggcgggcagaattcctgaacgctctaggataaagttcctaaagttttgcaacatgctttgactggtgaagtctggcagaccctgttgcagggttttaagcaggcgttcggggaagataatgtccgccaggtgcgcggccacggagcgctcgttgaaggccgtccataggtccttcaagttttgctttagcagcttctgcagctcctttaggttgcgctcctccaggcattgctgccacacgcccatggccgtttgccaggtgtagcacagaaataagtaaacgcagtcgcggacgtagtcgcggcgcgcctcgcccttgagcgtggaatgaagcacgttttgcccgaggcggttttcgtgcaaaattccaaggtaggagaccaggttgcagagctccacgttggaaattttgcaggcctggcgcacgtagccctggcgaaaggtgtagtgcaacgtttcctctagcttgcgctgcatctccgggtcagcaaagaaccgctgcatgcactcaagctccacggtaacaagcactgcggccatcattagcttgcgtcgctcctccaagtcggcaggctcgcgcgtctcaagccagcgcgccagctgctcatcgccaactgcgggtaggccctcctcggtttgttcttgcaagtttgcatccctctccaggggtcgtgcacggcgcacgatcagctcgctcatgactgtgctcataaccttggggggtaggttaagtgccgggtaggcaaagtgggtgacctcgatgctgcgtttcagcacggctaggcgcgcgttgtcaccctcaagttccaccagcactccacagtgactttcattttcgctgttttcttgttgcag

SEQ ID NO: 4 - DNA, 1590 bp, synthetic construct
  misc_feature 1..1590: note = Synthetic: Codon optimized Ad2 E2a coding sequence
  source 1..1590: mol_type = other DNA
atggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagagactgcggagacgcctggagtccgaggacgaggaagacagttcccaggacgctctggtgcctaggacaccatctccaagaccctccacttccaccgccgacctggccattgccagcaaaaaaaagaagaagaggccctcccctaaacctgaacgaccacccagcccagaagtgatcgtggactcagaggaagagcgcgaggacgtcgctctgcagatggtgggattcagcaatccacctgtgctcatcaagcacggcaaagggggaaaacggaccgtgcgcaggctgaacgaggatgatcctgtggcaaggggcatgaggacacaggaggagaaggaagaatcctccgaggccgagagcgagagcacagtgattaatccactgtccctgccaattgtgagcgcttgggagaagggaatggaggccgcccgggccctgatggacaagtaccacgtggacaacgacctgaaggctaatttcaaactgctgcctgaccaggtggaggccctggctgccgtgtgcaagacatggctgaatgaggagcatagagggctgcagctgacctttacatcaaacaagacctttgtgaccatgatgggcaggtttctgcaggcctacctgcaatctttcgccgaagtgacttacaagcaccacgagcccaccggctgcgccctgtggctgcacagatgcgccgagattgagggcgagctgaaatgcctgcacggctctatcatgattaacaaggagcacgtgatcgagatggatgtgacttccgaaaacggccagcgcgccctgaaggagcagtccagcaaggccaagatcgtgaagaacaggtggggcaggaacgtggtgcagatttctaacacagacgccagatgttgcgtgcatgacgccgcctgccctgccaatcagtttagcggcaagagctgcggaatgttcttcagcgagggagccaaggcgcaagtggccttcaagcagatcaaggccttcatgcaagccctgtatccaaatgcccagaccggccacggccacctcctgatgcccctgcgctgcgaatgtaactccaagcccggccacgcccctttcctgggcaggcagctgccaaaactgaccccttttgccctgagcaacgccgaagatctggatgccgatctgatcagcgacaagtccgtgctggcctccgttcaccaccccgccctgatcgtgttccagtgctgcaatcccgtgtacaggaatagcagagctcagggtggaggccccaactgcgacttcaagatctccgccccagatctgctgaacgctctcgtgatggtgcggagcctgtggtctgagaacttcacagagctgccccgcatggtggtgcccgagttcaagtggagcactaagcatcagtaccggaatgtgtctctgcccgtggcccactctgacgccagacagaatccttttgacttctga

SEQ ID NO: 5 - protein, 529 aa, synthetic construct
  REGION 1..529: note = Synthetic: Wild-type Ad2 E2a amino acid sequence
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 6 - DNA, 7686 bp, synthetic construct
  misc_feature 1..7686: note = Synthetic: mini-pHelper plasmid sequence
  source 1..7686: mol_type = other DNA
ggatccatcgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtactcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgatcatatggtttaaacgtcgacgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcctacatcgattctagtgaatccacagaaactagcgaggtaagcacttactctatgtcttttacatggtcctgggaaagtggaaaatacaccactgaaacttttgctaccaactcttacaccttctcctacattgcccaggaataaagaatcgtgaacctgttgcatgttatgtttcaacgtgtttatttttcaattgcagaaaatttcaagtcatttttcattcagtagtatagccccaccaccacatagcttatattgatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcgcttaagttcatgtcgctgtccagctgctgagccacaggctgctgtccaacttgcggttgctcaacgggcggcgaaggggaagtccacgcgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctggtcgcttagctcgctctgtgtagtagttgtagtatatccactctctcaaagcatccaggcgcctaggcaaaatagcaccctcccgctccagaacaacatacagcgcttccacagcggcagccataacagtcagccttaccagtaaaaaaacctattaaaaaacaccactcgacacggcaccagctcaatcagtcacagtgtaaaaagggccaagtacagagcgagtatatataggactaaaaaatgacgtaacggttaaagtccacaaaaaacacccagaaaaccgcacgcgaacctacgcccagaaacgaaagccaaaaaacccacaacttcctcaaatcttcacttccgttttcccacgatacgtcacttcccattttaaaaaaactacaattcccaatacatgcaagttactccgccctaaaacgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttcgaggagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttgctgaagagagcctccgcgtcttccagcgtgcgccgaagctgatcttcgcttttgtgatacaggcagctgcgggtgagggagcgcagagacctgttttttattttcagctcttgttcttggcccctgctttgttgaaatatagcatacagagtgggaaaaatcctatttctaagctcgcgggtcgatacgggttcgttgggcgccagacgcagcgctcctcctcctgctgctgccgccgctgtggatttcttgggctttgtcagagtcttgctatccggtcgcctttgcttctgtgtgaccgctgctgttgctgccgctgccgctgccgccggtgcagtaggggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccacgagcggtagccttggcgctgttgttgctcttgggctaacggcggcggctgcttagacttaccggccctggttccagtggtgtcccatctacggttgggtcggcgaacaggcagtgccggcggcgcctgaggagcggaggttgtagcgatgctgggaacggttgccaatttctggggcgccggcgaggggaatgcgaccgagggtgacggtgtttcgtctgacacctcttcggcctcggaagcttcgtctaggctgtcccagtcttccatcatctcctcctcctcgtccaaaacctcctctgcctgactgtcccagtattcctcctcgtccgtgggtggcggcggcggcagctgcagcttctttttgggtgccatcctgggaagcaagggcccgcggctgctgatagggctgcggcggcggggggattgggttgagctcctcgccggactgggggtccaggtaaaccccccgtccctttcgtagcagaaactcttggcgggctttgttgatggcttgcaattggccaaggatgtggccctgggtaatgacgcaggcggtaagctccgcatttggcgggcgggattggtcttcgtagaacctaatctcgtgggcgtggtagtcctcagggagaaggaaatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagagactgcggagacgcctggagtccgaggacgaggaagacagttcccaggacgctctggtgcctaggacaccatctccaagaccctccacttccaccgccgacctggccattgccagcaaaaaaaagaagaagaggccctcccctaaacctgaacgaccacccagcccagaagtgatcgtggactcagaggaagagcgcgaggacgtcgctctgcagatggtgggattcagcaatccacctgtgctcatcaagcacggcaaagggggaaaacggaccgtgcgcaggctgaacgaggatgatcctgtggcaaggggcatgaggacacaggaggagaaggaagaatcctccgaggccgagagcgagagcacagtgattaatccactgtccctgccaattgtgagcgcttgggagaagggaatggaggccgcccgggccctgatggacaagtaccacgtggacaacgacctgaaggctaatttcaaactgctgcctgaccaggtggaggccctggctgccgtgtgcaagacatggctgaatgaggagcatagagggctgcagctgacctttacatcaaacaagacctttgtgaccatgatgggcaggtttctgcaggcctacctgcaatctttcgccgaagtgacttacaagcaccacgagcccaccggctgcgccctgtggctgcacagatgcgccgagattgagggcgagctgaaatgcctgcacggctctatcatgattaacaaggagcacgtgatcgagatggatgtgacttccgaaaacggccagcgcgccctgaaggagcagtccagcaaggccaagatcgtgaagaacaggtggggcaggaacgtggtgcagatttctaacacagacgccagatgttgcgtgcatgacgccgcctgccctgccaatcagtttagcggcaagagctgcggaatgttcttcagcgagggagccaaggcgcaagtggccttcaagcagatcaaggccttcatgcaagccctgtatccaaatgcccagaccggccacggccacctcctgatgcccctgcgctgcgaatgtaactccaagcccggccacgcccctttcctgggcaggcagctgccaaaactgaccccttttgccctgagcaacgccgaagatctggatgccgatctgatcagcgacaagtccgtgctggcctccgttcaccaccccgccctgatcgtgttccagtgctgcaatcccgtgtacaggaatagcagagctcagggtggaggccccaactgcgacttcaagatctccgccccagatctgctgaacgctctcgtgatggtgcggagcctgtggtctgagaacttcacagagctgccccgcatggtggtgcccgagttcaagtggagcactaagcatcagtaccggaatgtgtctctgcccgtggcccactctgacgccagacagaatccttttgacttctgaacggcgcagacggcaagggtggggggtaaataatcacccgagagtgtacaaataaaaacatttgcctttattgaaagtgtctcctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttaagcatggagttgggtacc

SEQ ID NO: 7 - DNA, 453 bp, synthetic construct
  misc_feature 1..453: note = Synthetic: Codon optimized Ad2 E4orf6/7 coding sequence
  source 1..453: mol_type = other DNA
atgaccacaagcggcgtgccctttggcatgaccctgaggccaaccaggagcaggctgtccagacgcacaccatacagcagggaccggctgcctcctttcgagacagagaccagagccactatcctcgaggatcatcctctgctgcctgagtgcaatacactgaccatgcacaatgcctggacctccccttccccccccgtggaacagccacaggtgggacagcagcctgtggcccagcagctggacagcgacatgaacctgagcgaactgcccggcgagttcatcaatatcaccgacgagagactggccagacaggagaccgtgtggaatatcacccccaaaaatatgagcgtgacccacgacatgatgctgttcaaagccagccggggcgagagaaccgtgtacagcgtgtgctgggagggcggcggaagactgaatactagggtgctgtga

SEQ ID NO: 8 - protein, 150 aa, synthetic construct
  REGION 1..150: note = Synthetic: Wild-type Ad2 E4orf6/7 amino acid sequence
MTTSGVPFGMTLRPTRSRLSRRTPYSRDRLPPFETETRATILEDHPLLPECNTLTMHNAWTSPSPPVEQPQVGQQPVAQQLDSDMNLSELPGEFINITDERLARQETVWNITPKNMSVTHDMMLFKASRGERTVYSVCWEGGGRLNTRVL

SEQ ID NO: 9 - DNA, 7674 bp, synthetic construct
  misc_feature 1..7674: note = Synthetic: Mini pHelper 1.0
  source 1..7674: mol_type = other DNA
tttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcctacatcgattctagtgaatccacagaaactagcgaggtaagcacttactctatgtcttttacatggtcctgggaaagtggaaaatacaccactgaaacttttgctaccaactcttacaccttctcctacattgcccaggaataaagaatcgtgaacctgttgcatgttatgtttcaacgtgtttatttttcaattgcagaaaatttcaagtcatttttcattcagtagtatagccccaccaccacatagcttatattgatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcgcttaagttcatgtcgctgtccagctgctgagccacaggctgctgtccaacttgcggttgctcaacgggcggcgaaggggaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgtcgcttagctcgctctgtgtagtagttgtagtatatccactctctcaaagcatccaggcgcctaggcaaaatagcaccctcccgctccagaacaacatacagcgcttccacagcggcagccataacagtcagccttaccagtaaaaaaacctattaaaaaacaccactcgacacggcaccagctcaatcagtcacagtgtaaaaagggccaagtacagagcgagtatatataggactaaaaaatgacgtaacggttaaagtccacaaaaaacacccagaaaaccgcacgcgaacctacgcccagaaacgaaagccaaaaaacccacaacttcctcaaatcttcacttccgttttcccacgatacgtcacttcccattttaaaaaaactacaattcccaatacatgcaagttactccgccctaaaacgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttcgaggagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttgctgaagagagcctccgcgtcttccagcgtgcgccgaagctgatcttcgcttttgtgatacaggcagctgcgggtgagggagcgcagagacctgttttttattttcagctcttgttcttggcccctgctttgttgaaatatagcatacagagtgggaaaaatcctatttctaagctcgcgggtcgatacgggttcgttgggcgccagacgcagcgctcctcctcctgctgctgccgccgctgtggatttcttgggctttgtcagagtcttgctatccggtcgcctttgcttctgtgtgaccgctgctgttgctgccgctgccgctgccgccggtgcagtaggggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccacgagcggtagccttggcgctgttgttgctcttgggctaacggcggcggctgcttagacttaccggccctggttccagtggtgtcccatctacggttgggtcggcgaacaggcagtgccggcggcgcctgaggagcggaggttgtagcgatgctgggaacggttgccaatttctggggcgccggcgaggggaatgcgaccgagggtgacggtgtttcgtctgacacctcttcggcctcggaagcttcgtctaggctgtcccagtcttccatcatctcctcctcctcgtccaaaacctcctctgcctgactgtcccagtattcctcctcgtccgtgggtggcggcggcggcagctgcagcttctttttgggtgccatcctgggaagcaagggcccgcggctgctgatagggctgcggcggcggggggattgggttgagctcctcgccggactgggggtccaggtaaaccccccgtccctttcgtagcagaaactcttggcgggctttgttgatggcttgcaattggccaaggatgtggccctgggtaatgacgcaggcggtaagctccgcatttggcgggcgggattggtcttcgtagaacctaatctcgtgggcgtggtagtcctcagggagaaggaaatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagagactgcggagacgcctggagtccgaggacgaggaagacagttcccaggacgctctggtgcctaggacaccatctccaagaccctccacttccaccgccgacctggccattgccagcaaaaaaaagaagaagaggccctcccctaaacctgaacgaccacccagcccagaagtgatcgtggactcagaggaagagcgcgaggacgtcgctctgcagatggtgggattcagcaatccacctgtgctcatcaagcacggcaaagggggaaaacggaccgtgcgcaggctgaacgaggatgatcctgtggcaaggggcatgaggacacaggaggagaaggaagaatcctccgaggccgagagcgagagcacagtgattaatccactgtccctgccaattgtgagcgcttgggagaagggaatggaggccgcccgggccctgatggacaagtaccacgtggacaacgacctgaaggctaatttcaaactgctgcctgaccaggtggaggccctggctgccgtgtgcaagacatggctgaatgaggagcatagagggctgcagctgacctttacatcaaacaagacctttgtgaccatgatgggcaggtttctgcaggcctacctgcaatctttcgccgaagtgacttacaagcaccacgagcccaccggctgcgccctgtggctgcacagatgcgccgagattgagggcgagctgaaatgcctgcacggctctatcatgattaacaaggagcacgtgatcgagatggatgtgacttccgaaaacggccagcgcgccctgaaggagcagtccagcaaggccaagatcgtgaagaacaggtggggcaggaacgtggtgcagatttctaacacagacgccagatgttgcgtgcatgacgccgcctgccctgccaatcagtttagcggcaagagctgcggaatgttcttcagcgagggagccaaggcgcaagtggccttcaagcagatcaaggccttcatgcaagccctgtatccaaatgcccagaccggccacggccacctcctgatgcccctgcgctgcgaatgtaactccaagcccggccacgcccctttcctgggcaggcagctgccaaaactgaccccttttgccctgagcaacgccgaagatctggatgccgatctgatcagcgacaagtccgtgctggcctccgttcaccaccccgccctgatcgtgttccagtgctgcaatcccgtgtacaggaatagcagagctcagggtggaggccccaactgcgacttcaagatctccgccccagatctgctgaacgctctcgtgatggtgcggagcctgtggtctgagaacttcacagagctgccccgcatggtggtgcccgagttcaagtggagcactaagcatcagtaccggaatgtgtctctgcccgtggcccactctgacgccagacagaatccttttgacttctgaacggcgcagacggcaagggtggggggtaaataatcacccgagagtgtacaaataaaaacatttgcctttattgaaagtgtctcctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttaagcatggagttgggtaccggatccatcgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgattgacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgatcatatggtttaaacgtcgacgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctcct

SEQ ID NO: 10 - DNA, 12968 bp, synthetic construct
  misc_feature 1..12968: note = Synthetic: Mini-pHelper-AAV2
  source 1..12968: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgttcagaagtcaaaaggattctgtctggcgtcagagtgggccacgggcagagacacattccggtactgatgcttagtgctccacttgaactcgggcaccaccatgcggggcagctctgtgaagttctcagaccacaggctccgcaccatcacgagagcgttcagcagatctggggcggagatcttgaagtcgcagttggggcctccaccctgagctctgctattcctgtacacgggattgcagcactggaacacgatcagggcggggtggtgaacggaggccagcacggacttgtcgctgatcagatcggcatccagatcttcggcgttgctcagggcaaaaggggtcagttttggcagctgcctgcccaggaaaggggcgtggccgggcttggagttacattcgcagcgcaggggcatcaggaggtggccgtggccggtctgggcatttggatacagggcttgcatgaaggccttgatctgcttgaaggccacttgcgccttggctccctcgctgaagaacattccgcagctcttgccgctaaactgattggcagggcaggcggcgtcatgcacgcaacatctggcgtctgtgttagaaatctgcaccacgttcctgccccacctgttcttcacgatcttggccttgctggactgctccttcagggcgcgctggccgttttcggaagtcacatccatctcgatcacgtgctccttgttaatcatgatagagccgtgcaggcatttcagctcgccctcaatctcggcgcatctgtgcagccacagggcgcagccggtgggctcgtggtgcttgtaagtcacttcggcgaaagattgcaggtaggcctgcagaaacctgcccatcatggtcacaaaggtcttgtttgatgtaaaggtcagctgcagccctctatgctcctcattcagccatgtcttgcacacggcagccagggcctccacctggtcaggcagcagtttgaaattagccttcaggtcgttgtccacgtggtacttgtccatcagggcccgggcggcctccattcccttctcccaagcgctcacaattggcagggacagtggattaatcactgtgctctcgctctcggcctcggaggattcttccttctcctcctgtgtcctcatgccccttgccacaggatcatcctcgttcagcctgcgcacggtccgttttccccctttgccgtgcttgatgagcacaggtggattgctgaatcccaccatctgcagagcgacgtcctcgcgctcttcctctgagtccacgatcacttctgggctgggtggtcgttcaggtttaggggagggcctcttcttcttttttttgctggcaatggccaggtcggcggtggaagtggagggtcttggagatggtgtcctaggcaccagagcgtcctgggaactgtcttcctcgtcctcggactccaggcgtctccgcagtctcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctccctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagagcggccgcggtcctgtattagaggtcaacgtgagtgttttgcgacattttgcgacaccatgtggtcacgctgaggtatatatggccgagtgagcgagcaggatgcaacctccattttgaccgcgaaatttgaacgagcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggcaccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtacgtaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtaccagcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaacaattgcttgttaatcaataaaccgtttaattcgtttcagttgaactttggtctctgcgtatttctttcttatctagtttccatgctctaggatccactagtaacggccgccagtgtgctggaattcggctttgtagttaatgattaacccgccatgctacttatctacgtagccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatctatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagtacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 11 - DNA, 11148 bp, synthetic construct
  misc_feature 1..11148: note = Synthetic: Mini-pHelper- CMV-EGFP
  source 1..11148: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagctagcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgtacggatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgccaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagtacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatccggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgttcagaagtcaaaaggattctgtctggcgtcagagtgggccacgggcagagacacattccggtactgatgcttagtgctccacttgaactcgggcaccaccatgcggggcagctctgtgaagttctcagaccacaggctccgcaccatcacgagagcgttcagcagatctggggcggagatcttgaagtcgcagttggggcctccaccctgagctctgctattcctgtacacgggattgcagcactggaacacgatcagggcggggtggtgaacggaggccagcacggacttgtcgctgatcagatcggcatccagatcttcggcgttgctcagggcaaaaggggtcagttttggcagctgcctgcccaggaaaggggcgtggccgggcttggagttacattcgcagcgcaggggcatcaggaggtggccgtggccggtctgggcatttggatacagggcttgcatgaaggccttgatctgcttgaaggccacttgcgccttggctccctcgctgaagaacattccgcagctcttgccgctaaactgattggcagggcaggcggcgtcatgcacgcaacatctggcgtctgtgttagaaatctgcaccacgttcctgccccacctgttcttcacgatcttggccttgctggactgctccttcagggcgcgctggccgttttcggaagtcacatccatctcgatcacgtgctccttgttaatcatgatagagccgtgcaggcatttcagctcgccctcaatctcggcgcatctgtgcagccacagggcgcagccggtgggctcgtggtgcttgtaagtcacttcggcgaaagattgcaggtaggcctgcagaaacctgcccatcatggtcacaaaggtcttgtttgatgtaaaggtcagctgcagccctctatgctcctcattcagccatgtcttgcacacggcagccagggcctccacctggtcaggcagcagtttgaaattagccttcaggtcgttgtccacgtggtacttgtccatcagggcccgggcggcctccattcccttctcccaagcgctcacaattggcagggacagtggattaatcactgtgctctcgctctcggcctcggaggattcttccttctcctcctgtgtcctcatgccccttgccacaggatcatcctcgttcagcctgcgcacggtccgttttccccctttgccgtgcttgatgagcacaggtggattgctgaatcccaccatctgcagagcgacgtcctcgcgctcttcctctgagtccacgatcacttctgggctgggtggtcgttcaggtttaggggagggcctcttcttcttttttttgctggcaatggccaggtcggcggtggaagtggagggtcttggagatggtgtcctaggcaccagagcgtcctgggaactgtcttcctcgtcctcggactccaggcgtctccgcagtctcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctccctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgttt

SEQ ID NO: 12 - DNA, 15719 bp, synthetic construct
  misc_feature 1..15719: note = Synthetic: pAAVone-AAV2-CMV-EGFP
  source 1..15719: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagctagcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgtacggatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgccaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagtacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatccggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgttcagaagtcaaaaggattctgtctggcgtcagagtgggccacgggcagagacacattccggtactgatgcttagtgctccacttgaactcgggcaccaccatgcggggcagctctgtgaagttctcagaccacaggctccgcaccatcacgagagcgttcagcagatctggggcggagatcttgaagtcgcagttggggcctccaccctgagctctgctattcctgtacacgggattgcagcactggaacacgatcagggcggggtggtgaacggaggccagcacggacttgtcgctgatcagatcggcatccagatcttcggcgttgctcagggcaaaaggggtcagttttggcagctgcctgcccaggaaaggggcgtggccgggcttggagttacattcgcagcgcaggggcatcaggaggtggccgtggccggtctgggcatttggatacagggcttgcatgaaggccttgatctgcttgaaggccacttgcgccttggctccctcgctgaagaacattccgcagctcttgccgctaaactgattggcagggcaggcggcgtcatgcacgcaacatctggcgtctgtgttagaaatctgcaccacgttcctgccccacctgttcttcacgatcttggccttgctggactgctccttcagggcgcgctggccgttttcggaagtcacatccatctcgatcacgtgctccttgttaatcatgatagagccgtgcaggcatttcagctcgccctcaatctcggcgcatctgtgcagccacagggcgcagccggtgggctcgtggtgcttgtaagtcacttcggcgaaagattgcaggtaggcctgcagaaacctgcccatcatggtcacaaaggtcttgtttgatgtaaaggtcagctgcagccctctatgctcctcattcagccatgtcttgcacacggcagccagggcctccacctggtcaggcagcagtttgaaattagccttcaggtcgttgtccacgtggtacttgtccatcagggcccgggcggcctccattcccttctcccaagcgctcacaattggcagggacagtggattaatcactgtgctctcgctctcggcctcggaggattcttccttctcctcctgtgtcctcatgccccttgccacaggatcatcctcgttcagcctgcgcacggtccgttttccccctttgccgtgcttgatgagcacaggtggattgctgaatcccaccatctgcagagcgacgtcctcgcgctcttcctctgagtccacgatcacttctgggctgggtggtcgttcaggtttaggggagggcctcttcttcttttttttgctggcaatggccaggtcggcggtggaagtggagggtcttggagatggtgtcctaggcaccagagcgtcctgggaactgtcttcctcgtcctcggactccaggcgtctccgcagtctcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctccctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagagcggccgcggtcctgtattagaggtcaacgtgagtgttttgcgacattttgcgacaccatgtggtcacgctgaggtatatatggccgagtgagcgagcaggatgcaacctccattttgaccgcgaaatttgaacgagcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggcaccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtacgtaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtaccagcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaacaattgcttgttaatcaataaaccgtttaattcgtttcagttgaactttggtctctgcgtatttctttcttatctagtttccatgctctaggatccactagtaacggccgccagtgtgctggaattcggctttgtagttaatgattaacccgccatgctacttatctacgtagccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatctatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgttt

SEQ ID NO: 13 - DNA, 10582 bp, synthetic construct
  misc_feature 1..10582: note = Synthetic: Mini-pHelper-Rep
  source 1..10582: mol_type = other DNA
tttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcctacatcgatagatctgcagaattcggcttggcggctgcgcgttcaaacctcccgcttcaaaatggagaccctgcgtgctcactcgggcttaaatacccagcgtgaccacatggtgtcgcaaaatgtcgcaaaacactcacgtgacctctaatacaggacctctagagcatggctacaaatcatttattgttcaaagatgcagtcatccaaatccacattgaccagatcgcaggcagtgcaagcgtctggcacctttcccatgatatgatgaatgtagcacagtttctgatacgcctttttgacgacagaaacgggttgagattctgacacgggaaagcactctaaacagtctttctgtccgtgagtgaagcagatatttgaattctgattcattctctcgcattgtctgcagggaaacagcatcagattcatgcccacgtgacgagaacatttgtttacgtacctgtctgcgtagttgatcgaagcttccgcgtctgacgtcgatggctgcgcaactgactcgcgcacccgtttgggctcacttatatctgcgtcactgggggcgggtcttttcttggctccaccctttttgacgtagaattcatgctccacctcaaccacgtgatcctttgcccaccggaaaaagtctttgacttcctgcttggtgaccttcccaaagtcatgatccagacggcgggtgagttcaaatttgaacatccggtcttgcaacggctgctggtgttcgaaggtcgttgagttcccgtcaatcacggcgcacatgttggtgttggaggtgacgatcacgggagtcgggtctatctgggccgaggacttgcatttctggtccacgcgcaccttgcttcctccgagaatggctttggccgactccacgaccttggcggtcatcttcccctcctcccaccagatcaccatcttgtcgacacagtcgttgaagggaaagttctcattggtccagtttacgcacccgtagaagggcacagtgtgggctatggcctccgcgatgttggtcttcccggtagttgcaggcccaaacagccagatggtgttcctcttgccgaactttttcgtggcccatcccagaaagacggaagccgcatattggggatcgtacccgtttagttccaaaattttataaatccgattgctggaaatgtcctccacgggctgctggcccaccaggtagtcgggggcggttttagtcaggctcataatctttcccgcattgtccaaggcagccttgatttgggaccgcgagttggaggccgcattgaaggagatgtatgaggcctggtcctcctggatccactgcttctccgaggtaatccccttgtccacgagccacccgaccagctccatgtacctggctgaagtttttgatctgatcaccggcgcatcagaattgggattctgattctctttgttctgctcctgcgtctgcgacacgtgcgtcagatgctgcgccaccaaccgtttacgctccgtgagattcaaacaggcgcttaaatactgttccatattagtccacgcccactggagctcaggctgggttttggggagcaagtaattggggatgtagcactcatccaccaccttgttcccgcctccggcgccatttctggtctttgtgaccgcgaaccagtttggcaaagtcggctcgatcccgcggtaaattctctgaatcagtttttcgcgaatctgactcaggaaacgtcccaaaaccatggatttcaccccggtggtttccacgagcacgtgcatgtggaagtagctctctcccttctcaaattgcacaaagaaaagggcctccggtgccttactcacacggcgccattccgtcagaaagtcgcgctgcagcttctcggccacggtcaggggtgcctgctcaatcagattcagatccatgtcagaatctggcggcaactcccattccttctcggccacccagttcacaaagctgtcagaaatgccgggcagatgctcgtcaaggtcgctggggaccttaatcacaatctcgtaaaaccccggcatggcggctgctcgttcaaatttcgcggtcaaaatggaggttgcatcctgctcgctcactcggccatatatacctcagcgtgaccacatggtgtcgcaaaatgtcgcaaaacactcacgttgacctctaatacaggaccgcggccgctctagtgaatccacagaaactagcgaggtaagcacttactctatgtcttttacatggtcctgggaaagtggaaaatacaccactgaaacttttgctaccaactcttacaccttctcctacattgcccaggaataaagaatcgtgaacctgttgcatgttatgtttcaacgtgtttatttttcaattgcagaaaatttcaagtcatttttcattcagtagtatagccccaccaccacatagcttatattgatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcgcttaagttcatgtcgctgtccagctgctgagccacaggctgctgtccaacttgcggttgctcaacgggcggcgaaggggaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgtcgcttagctcgctctgtgtagtagttgtagtatatccactctctcaaagcatccaggcgcctaggcaaaatagcaccctcccgctccagaacaacatacagcgcttccacagcggcagccataacagtcagccttaccagtaaaaaaacctattaaaaaacaccactcgacacggcaccagctcaatcagtcacagtgtaaaaagggccaagtacagagcgagtatatataggactaaaaaatgacgtaacggttaaagtccacaaaaaacacccagaaaaccgcacgcgaacctacgcccagaaacgaaagccaaaaaacccacaacttcctcaaatcttcacttccgttttcccacgatacgtcacttcccattttaaaaaaactacaattcccaatacatgcaagttactccgccctaaaacgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttcgaggagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttgctgaagagagcctccgcgtcttccagcgtgcgccgaagctgatcttcgcttttgtgatacaggcagctgcgggtgagggagcgcagagacctgttttttattttcagctcttgttcttggcccctgctttgttgaaatatagcatacagagtgggaaaaatcctatttctaagctcgcgggtcgatacgggttcgttgggcgccagacgcagcgctcctcctcctgctgctgccgccgctgtggatttcttgggctttgtcagagtcttgctatccggtcgcctttgcttctgtgtgaccgctgctgttgctgccgctgccgctgccgccggtgcagtaggggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccacgagcggtagccttggcgctgttgttgctcttgggctaacggcggcggctgcttagacttaccggccctggttccagtggtgtcccatctacggttgggtcggcgaacaggcagtgccggcggcgcctgaggagcggaggttgtagcgatgctgggaacggttgccaatttctggggcgccggcgaggggaatgcgaccgagggtgacggtgtttcgtctgacacctcttcggcctcggaagcttcgtctaggctgtcccagtcttccatcatctcctcctcctcgtccaaaacctcctctgcctgactgtcccagtattcctcctcgtccgtgggtggcggcggcggcagctgcagcttctttttgggtgccatcctgggaagcaagggcccgcggctgctgatagggctgcggcggcggggggattgggttgagctcctcgccggactgggggtccaggtaaaccccccgtccctttcgtagcagaaactcttggcgggctttgttgatggcttgcaattggccaaggatgtggccctgggtaatgacgcaggcggtaagctccgcatttggcgggcgggattggtcttcgtagaacctaatctcgtgggcgtggtagtcctcagggagaaggaaatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagagactgcggagacgcctggagtccgaggacgaggaagacagttcccaggacgctctggtgcctaggacaccatctccaagaccctccacttccaccgccgacctggccattgccagcaaaaaaaagaagaagaggccctcccctaaacctgaacgaccacccagcccagaagtgatcgtggactcagaggaagagcgcgaggacgtcgctctgcagatggtgggattcagcaatccacctgtgctcatcaagcacggcaaagggggaaaacggaccgtgcgcaggctgaacgaggatgatcctgtggcaaggggcatgaggacacaggaggagaaggaagaatcctccgaggccgagagcgagagcacagtgattaatccactgtccctgccaattgtgagcgcttgggagaagggaatggaggccgcccgggccctgatggacaagtaccacgtggacaacgacctgaaggctaatttcaaactgctgcctgaccaggtggaggccctggctgccgtgtgcaagacatggctgaatgaggagcatagagggctgcagctgacctttacatcaaacaagacctttgtgaccatgatgggcaggtttctgcaggcctacctgcaatctttcgccgaagtgacttacaagcaccacgagcccaccggctgcgccctgtggctgcacagatgcgccgagattgagggcgagctgaaatgcctgcacggctctatcatgattaacaaggagcacgtgatcgagatggatgtgacttccgaaaacggccagcgcgccctgaaggagcagtccagcaaggccaagatcgtgaagaacaggtggggcaggaacgtggtgcagatttctaacacagacgccagatgttgcgtgcatgacgccgcctgccctgccaatcagtttagcggcaagagctgcggaatgttcttcagcgagggagccaaggcgcaagtggccttcaagcagatcaaggccttcatgcaagccctgtatccaaatgcccagaccggccacggccacctcctgatgcccctgcgctgcgaatgtaactccaagcccggccacgcccctttcctgggcaggcagctgccaaaactgaccccttttgccctgagcaacgccgaagatctggatgccgatctgatcagcgacaagtccgtgctggcctccgttcaccaccccgccctgatcgtgttccagtgctgcaatcccgtgtacaggaatagcagagctcagggtggaggccccaactgcgacttcaagatctccgccccagatctgctgaacgctctcgtgatggtgcggagcctgtggtctgagaacttcacagagctgccccgcatggtggtgcccgagttcaagtggagcactaagcatcagtaccggaatgtgtctctgcccgtggcccactctgacgccagacagaatccttttgacttctgaacggcgcagacggcaagggtggggggtaaataatcacccgagagtgtacaaataaaaacatttgcctttattgaaagtgtctcctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttaagcatggagttgggtaccggatccatcgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtactcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgatcatatggtttaaacgtcgacgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctcct

SEQ ID NO: 14 - DNA, 131 bp, synthetic construct
  misc_feature 1..131: note = Synthetic: Wild-type AAV2 P5 promoter (P5-AAV2)
  source 1..131: mol_type = other DNA
ggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 15 - DNA, 137 bp, synthetic construct
  misc_feature 1..137: note = Synthetic: Modified AAV2 P5 promoter (P5I)
  source 1..137: mol_type = other DNA
ggtcctgtattagaggtcaacgtgagtgttttgcgacattttgcgacaccatgtggtcacgctgaggtatatatggccgagtgagcgagcaggatgcaacctccattttgaccgcgaaatttgaacgagcagccgcc

SEQ ID NO: 16 - DNA, 414 bp, synthetic construct
  misc_feature 1..414: note = Synthetic: VA coding region of Ad2
  source 1..414: mol_type = other DNA
gggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagcccct

SEQ ID NO: 17 - DNA, 453 bp, synthetic construct
  misc_feature 1..453: note = Synthetic: Modified E4 coding region of mini-pHelper
  source 1..453: mol_type = other DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtga

SEQ ID NO: 18 - DNA, 1164 bp, synthetic construct
  misc_feature 1..1164: note = Synthetic: Modified E4 coding region of mini-pHelper1.0
  source 1..1164: mol_type = other DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtga

SEQ ID NO: 19 - DNA, 835 bp, synthetic construct
  misc_feature 1..835: note = Synthetic: Ad2 E4 promoter + E2a promoter sequence
  source 1..835: mol_type = other DNA
aacagtcagccttaccagtaaaaaaacctattaaaaaacaccactcgacacggcaccagctcaatcagtcacagtgtaaaaagggccaagtacagagcgagtatatataggactaaaaaatgacgtaacggttaaagtccacaaaaaacacccagaaaaccgcacgcgaacctacgcccagaaacgaaagccaaaaaacccacaacttcctcaaatcttcacttccgttttcccacgatacgtcacttcccattttaaaaaaactacaattcccaatacatgcaagttactccgccctaaaacgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttcgaggagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttgctgaagagagcctccgcgtcttccagcgtgcgccgaagctgatcttcgctttt

SEQ ID NO: 20 - DNA, 1863 bp, synthetic construct
  misc_feature 1..1863: note = Synthetic: AAV rep coding sequence
  source 1..1863: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggcaccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtacgtaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 21 - DNA, 2205 bp, synthetic construct
  misc_feature 1..2205: note = Synthetic: AAV2cap coding sequence
  source 1..2205: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtaccagcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctg

SEQ ID NO: 22 - DNA, 303 bp, synthetic construct
  misc_feature 1..303: note = Synthetic: Ad2 E4 promoter sequence
  source 1..303: mol_type = other DNA
gacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttgctgaagagagcctccgcgtcttccagcgtgcgccgaagctgatcttcgctttt

SEQ ID NO: 23 - DNA, 303 bp, synthetic construct
  misc_feature 1..303: note = Synthetic: Ad2 E2a promoter sequence
  source 1..303: mol_type = other DNA
gttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgtt

SEQ ID NO: 24 - DNA, 885 bp, synthetic construct
  misc_feature 1..885: note = Synthetic: Ad2 E4orf6 coding sequence
  source 1..885: mol_type = other DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtag

SEQ ID NO: 25 - DNA, 885 bp, synthetic construct
  misc_feature 1..885: note = Synthetic: Codon optimized Ad2 E4orf6 coding sequence
  source 1..885: mol_type = other DNA
atgacaacaagcggcgtgcccttcggcatgaccctgaggcctacaagaagccggctgagcagaagaaccccttacagcagagacagactgcctccattcgagacagagacacgggccaccatcctggaagatcaccctctgctgcccgagtgcaacaccctgacaatgcacaacgtgtcctacgtgcggggcctgccttgtagcgttggcttcacactgatccaagagtgggtcgtgccctgggacatggtgctgaccagagaggaactggtcatcctgcggaagtgtatgcacgtgtgcctgtgctgcgccaacatcgacatcatgaccagcatgatgatccacggctacgagagctgggccctgcactgtcactgttctagccctggcagcctgcagtgtattgctggtggacaggttctggccagctggttcagaatggtggtggacggcgccatgttcaaccagcggttcatctggtacagagaggtggtcaactacaacatgcccaaagaagtgatgttcatgagcagcgttttcatgcggggcagacacctgatctacctgcggctttggtacgatggccacgtgggatctgtggtgcctgccatgagctttggctacagcgccctgcattgcggcatcctgaacaacatcgtggtgctgtgctgcagctactgcgccgatctgagcgagatcagagtgcggtgttgtgccagacggaccagacggctgatgctgagagccgtgcggatcattgccgaagagacaaccgccatgctgtactcttgccggaccgagagaagaaggcagcagttcatcagagccctgctccagcaccaccgccctatcctgatgcacgattatgactctacccccatgtag

SEQ ID NO: 26 - protein, 294 aa, synthetic construct
  REGION 1..294: note = Synthetic: Wild-type Ad2 E4orf6 amino acid sequence
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

(19) United States
(12) Patent Application Publication
(10) Pub. No.: US 2024/0132911 A1
(43) Pub. Date: Apr. 25, 2024

(54) COMPOSITIONS AND METHODS FOR RECOMBINANT PARVOVIRUS PRODUCTION

(71) Applicant: AAVNERGENE INC., Rockville, MD (US)

(72) Inventors: Qizhao WANG, Rockville, MD (US); Daozhan YU, Rockville, MD (US); Yu LIU, Rockville, MD (US); Cuiping CHEN, Rockville, MD (US); Rongze YANG, Rockville, MD (US)

(21) Appl. No.: 18/347,891

(22) Filed: Jul. 5, 2023

Related U.S. Application Data
(63) Continuation-in-part of application No. 18/181,952, filed on Mar. 10, 2023.
(60) Provisional application No. 63/341,201, filed on May 12, 2022.

(51) Int. Cl. C12N 15/86; C12N 7/00; C12N 15/113

(52) U.S. Cl. CPC C12N 15/86; C12N 7/00; C12N 15/113; C12N 2710/10043; C12N 2750/14121; C12N 2750/14151

(57) ABSTRACT

Expression constructs and methods for recombinant parvovirus production are disclosed. In some embodiments, the expression construct encodes (1) adenovirus E4 and E2a proteins, (2) parvovirus proteins necessary for the production of the recombinant parvovirus, and (3) a recombinant parvovirus genome, thus allowing production of the recombinant parvovirus by transfecting a host cell with a single expression construct.

This application is a Continuation-In-Part (CIP) application of U.S. patent application Ser. No. 18/181,952, filed on Mar. 10, 2023, which claims priority from U.S. Provisional App. No. 63/341,201, filed May 12, 2022. The contents of these applications are expressly incorporated herein by reference for all purposes.

### FIELD

The present application relates generally to compositions and methods for producing recombinant viruses. More particularly, the application relates to compositions for production of recombinant parvoviruses, such as adeno-associated viruses (AAVs).

### BACKGROUND

Recombinant viruses, such as recombinant adeno-associated viruses (rAAV) have been developed as vectors by replacing all viral genes with a therapeutic transgene expression cassette, while retaining the only cis elements, the ITRs, which are required for vector packaging and DNA replication. Early methods of rAAV production relied on a plasmid system comprising: 1) an AAV helper plasmid (generally encompassing AAV Rep and Cap coding regions, while lacking AAV ITRs so it cannot replicate or package itself) and 2) an ITR-containing plasmid (generally encompassing a selected transgene of interest bounded by AAV ITRs which provides for viral replication and packaging functions). Both the helper plasmid and the ITR-containing plasmid bearing the selected gene can be introduced into suitable cells for production by transient transfection. The transfected cell can then be infected with a helper virus, such as an adenovirus or herpes simplex virus, which transactivates the AAV promoters present on the helper plasmid that direct the transcription and translation of AAV Rep and Cap regions. Regarding the Ad helper virus, the Ela, E1b, E2a, E4, and VA RNA genes can supply the helper functions necessary for rAAV production.

Infection of helper virus into producer cells to generate rAAV was effective in producing rAAV. However, a consequence is that it can also produce Ad helper virus particles that can elicit immune responses from the host. In certain platforms, the viral helper genes necessary for AAV manufacturing can be stably transfected into the manufacturing cell line (e.g., HEK293 cells), thereby reducing the possibility of an anti-helper virus immune response by the host immune system coming from trace level of residual helper virus.

More recently, a triple-plasmid transfection method has been developed. This method uses an AAV serotype-specific Rep and Cap plasmid as well as the transgene-containing plasmid but eliminates the use of helper virus infection by supplying the essential helper viral genes on a third plasmid (i.e., the viral coding sequences were removed or reduced), thus lowering the potential anti-helper virus immune response by the host immune system. Supplying the viral helper genes on the third plasmid greatly decreased helper viral production in the transfected cells, providing only rAAV. Multiplasmid transient transfection of adherent HEK293 cells is a commonly used method for rAAV production.

In a multiplasmid system, it is important to maintain an appropriate plasmid size and plasmid ratio to maximize transfection efficiency and AAV production. There is a need in the art for a simplified and efficient plasmid-based system for producing rAAVs, which provides for improved transfection and lowered immunogenicity, while still retaining optimum expression of the transgene. The present application is predicated on the discovery of dispensable adenovirus regions in traditional helper plasmids to provide such benefits.

### SUMMARY

One aspect of the present application relates to an expression construct for production of a recombinant parvovirus. The expression construct comprises: (1) a modified or unmodified adenovirus E4 coding region that encodes E4 proteins; (2) a modified or unmodified adenovirus E2a coding region that encodes an E2a protein; (3) an adenovirus VA RNA coding region that encodes adenovirus VAI and VAII RNA; (4) a parvovirus protein coding region that encodes parvovirus proteins necessary for the production of the recombinant parvovirus; (5) a recombinant parvovirus sequence comprising (a) an expression cassette comprising a nucleotide sequence encoding a transgene and a regulatory element operably linked to the nucleotide sequence, and (b) at least one ITR at one end of the expression cassette; and (6) one or more regulatory elements that allow expression of the E4 proteins the E2a protein, the adenovirus VA RNA, and the parvovirus proteins necessary for the production of the recombinant parvovirus in a host cell.

Another aspect of the present application relates to a polynucleotide expression construct for production of recombinant parvoviruses. The expression construct comprises (a) a modified adenovirus E4 coding region encoding adenovirus E4 proteins, wherein the modified adenovirus E4 coding region comprises (i) partial or complete deletion of E4orf6/7 intron 1, or (ii) a partial or complete deletion of E4orf6/7 intron 2, or both (i) and (ii); (b) a modified adenovirus E2a coding region encoding an adenovirus E2a protein, wherein the modified adenovirus E2a coding region comprises at least one deletion in E4orf6/7 intron 1 or E4orf6/7 intron 2; (c) a sequence encoding one or more adenovirus VA RNAs; and (d) one or more regulatory elements operably linked to (a), (b) and (c).

Another aspect of the present application relates to a method for producing a recombinant parvovirus particle using the expression construct of the present application. The method comprises the step of introducing the expression construct of the present application into a host cell, incubating the host cell harboring the expression construct for a desired period of time to produce recombinant parvovirus particles, and harvesting recombinant parvovirus particles after the incubation period.

### BRIEF DESCRIPTION OF THE DRAWINGS

FIG. 1 shows a map depicting the arrangement of adenovirus (Ad2) coding regions relative to various deletions in a series of exemplary expression cassettes of the present application. CoE2A: codon optimized E2A coding sequence.

FIG. 2 shows an expanded map depicting an Ad2 E4 coding region, the arrangement of Ad E4orfs in the coding region, and the deletions depicted in FIG. 1.

FIG. 3 shows an expanded map depicting an Ad2 E2a coding region, the Ad2 E2A promoter and Ad2 mRNAs expressed at early and late times in infection, and deletions depicted in FIG.

FIG. 4 shows a plasmid construct, mini-pHelper, that contains the corresponding arrangement of Ad helper genes shown in FIG. 1.

FIG. 5 shows a plasmid construct, mini-pHelper-1.0, that contains the corresponding arrangement of Ad helper genes shown in FIG. 1.

FIG. 6 shows the effects of various deletions and combination of deletions in FIG. 1 on the ability of the expression cassette to sufficiently provide helper functions for AAV packaging.

FIG. 7 is a composite of schematics showing mini-pHelper based triple-plasmid, dual-plasmid and single-plasmid virus production system.

FIG. 8 shows the package efficiency of mini-helper based AAV production system in adherent HEK 293T cells.

FIG. 9 shows the package efficiency of mini-helper based AAV production systems in suspension cultured HEK 293T cells.

FIG. 10 shows the package efficiency of mini-helper based dual-plasmid V2 system for different AAV serotypes.

FIG. 11 shows the use of mini-helper for AAV library production.

FIG. 12 shows an exemplary plasmid construct, pAAVone-AAV2-CMV-EGFP, for single-plasmid AAV production system.

FIG. 13 shows that a modified P5 promoter (PSI) increase AAV yield compared to wild-type AAV2 P5 promoter.

FIG. 14 shows the package efficiency of mini-helper 1.0 for different AAV serotypes.

FIG. 15 shows a list of nucleotide and amino acid sequences described in the instant application.

### DETAILED DESCRIPTION

### I. Definitions

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

As used herein, the term “polynucleotide” refers to polydeoxyribonucleotide, which may comprise unmodified DNA or modified DNA and may comprise both single- and double-stranded DNA.

As used herein, the term “heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector. As an example, “heterologous” when used in the context of nucleic acid sequences, such as coding sequences and control sequences, may refer to sequences that are not normally joined together, and/or are not normally associated with one another under ordinary circumstances found in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this application. Allelic variation or naturally occurring mutational events do not give rise to heterologous DNA, as used herein.

A “coding sequence,” as used herein, refers to a nucleic acid sequence which “encodes” a particular protein or functional nucleotide, such as a siRNA. The nucleic acid sequence in a polynucleotide is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence is usually located 3′ to the coding sequence.

A “coding region,” as used herein, refers to a region in a genomic DNA where a coding sequence for a gene is located. The coding region of a gene may include both the exon and intron sequences of the gene, as well as the regulatory sequences (e.g., promoter and enhances) for the gene. For example, an adenovirus E4 coding region may include nucleotide sequence 32645-35835 of the Ad2 genome, and an adenovirus E2a coding region may include nucleotide sequence 22233-27575 of the Ad2 genome.

A “modified coding region,” as used herein, refers to a coding region that have been modified by deletion, substitution and/or insertion of one or more nucleotides. For example, a modified adenovirus E4 coding region may include nucleotide sequence 32645-35835 of the Ad2 genome with a deletion of one or more nucleotides in E4orf6/7 intron 1 and/or intron 2, and a modified adenovirus E2a coding region may include nucleotide sequence 22233-27575 of the Ad2 genome with a deletion of one or more nucleotides in later-E2a intron 1 and/or intron 2.

The term “adenovirus E4 proteins” refers to proteins that are (1) produced by the adenovirus E4 region and (2) required for AAV production in host cell. Adenovirus E4 proteins may be encoded by different open reading frames in different adenoviruses. The term “adenovirus E4 proteins” as used herein, encompasses functional homologs and functional equivalents of adenovirus E4 proteins. Examples of the adenovirus E4 proteins include the protein encoded by E4 open reading frame 6 (E4orf6) E4 open reading frame 6/7 (E4orf6/7) and functional equivalents thereof.

The term “adenovirus E2a protein” refers to the protein encoded by the E2a region of an adenovirus genome. The adenovirus E2a protein is a DNA-binding protein that plays a role in the elongation phase of viral strand displacement replication by unwinding the template in an ATP-independent fashion. As used herein, the term “adenovirus E2a protein” encompasses functional homologs and functional equivalents of adenovirus E2a proteins.

As used herein, the term “adenovirus VA RNA” refers a type of non-coding RNA found in adenovirus that plays a role in regulating translation. VA RNAs include VAI and VAII. As used herein, the term “adenovirus VA RNA” encompasses functional homologs and functional equivalents of adenovirus VA RNA.

The term “adenovirus VA RNA coding region” refers to a region encoding adenovirus VA RNAs. An exemplary VA coding region includes nucleotide sequence 10426-11156 (SEQ ID NO:16) of the Ad2 genome.

The terms “regulatory element,” “regulatory sequence” and “regulatory region” are used with reference to promoters, enhancers, polyadenylation signals, transcription termination sequences, upstream regulatory domains, origins of replication, internal ribosome entry sites (“IRES”), and the like, which collectively provide for the replication, transcription, and translation of a coding sequence in a recipient cell. Not all of these regulatory sequences need always be present so long as the selected coding sequence is capable of being replicated, transcribed and translated in an appropriate host cell.

The terms “promoter” and “promoter region” are used herein with reference to a DNA regulatory sequence to which RNA polymerase binds, initiating transcription of a downstream (3′ direction) coding sequence. Further, these terms should be broadly construed as additionally encompassing other regulatory elements, including enhancer regions, intron splice donors and acceptors, other 5′ untranslated regions and the like. A promoter sequence may be homologous or heterologous to the desired gene sequence. A wide range of promoters are known and available in the art for the present application, including a wide range of viral and mammalian promoters. Cell type selective or tissue specific promoters can be utilized to target or enhance expression of gene sequences in specific cell populations relative to others. Suitable mammalian and viral promoters. A promoter may be constitutively active, conditionally active, or inducible, depending on the cell type.

The terms “enhancer” and “enhancer region” are used herein, denotes a polynucleotide sequence which in the cis location acts on the activity of a promoter and thus stimulates the transcription of a gene or coding sequence functionally connected to this promoter. Unlike promoters the effect of enhancers is independent of position and orientation and they can therefore be positioned in front of or behind a transcription unit, within an intron or even within the coding region. The enhancer may be located both in the immediate vicinity of the transcription unit and at a considerable distance from the promoter. It is also possible to have a physical and functional overlap with the promoter. The skilled artisan will be aware of a number of enhancers from various sources (and deposited in databanks such as GenBank, e.g. SV40 enhancers, CMV enhancers, polyoma enhancers, adenovirus enhancers) which are available as independent elements or elements cloned within polynucleotide sequences (e.g. deposited at the ATCC or from commercial and individual sources). A number of promoter sequences also contain enhancer sequences such as the frequently used CMV promoter. One example of an inducible enhancer is the metallothionein enhancer, which can be stimulated by glucocorticoids or heavy metals.

“Operably linked” refers to an arrangement of elements wherein the components so described are configured so as to perform their usual function. Thus, control sequences operably linked to a coding sequence are capable of effecting the expression of the coding sequence. Operably linking a heterologous sequence to a promoter, results in a chimeric gene. The control sequences need not be contiguous with the coding sequence, so long as they function to direct the expression thereof. Thus, for example, intervening untranslated yet transcribed sequences can be present between a promoter sequence and the coding sequence, and the promoter sequence can still be considered “operably linked” to the coding sequence.

For the purpose of describing the relative position of a nucleotide sequence in a particular nucleic acid molecule throughout the present application, such as when a particular nucleotide sequence is described as being situated “upstream”, “downstream”, “3”, or “5” relative to another sequence, these modifiers should be construed as relating sequence portions in the “sense” or “coding” strand of a DNA molecule, as is conventional in the art.

The term “transgene” refers to a polynucleotide that is introduced into a cell and is capable of being transcribed into RNA and optionally, translated and/or expressed under appropriate conditions. The transgene confers a desired property to a cell into which it was introduced, or otherwise leads to a desired therapeutic or diagnostic outcome. In another aspect, it may be transcribed into a molecule that mediates RNA interference, such as a miRNA, an siRNA, an shRNA, or a guide RNA for CRISPR/Cas9 mediated targeting of the mutant allele.

The term “expression” encompasses the processes by which nucleic acids (e.g., DNA) are transcribed to produce RNAs, such as mRNAs that are processed and translated into polypeptides.

The term “gene product” (also referred to herein as “gene expression product” or “expression product”) encompasses products resulting from expression of a gene, such as a mRNA transcribed from a gene and polypeptides arising from translation of the mRNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic nucleotide sequences, cDNA sequences, mRNA sequences, and polypeptide sequences from a variety of species, including human, are known in the art and are available in publicly accessible databases such as those available at the National Center for Biotechnology Information (www.ncbi.nih.gov) or the Universal Protein Resource (www.uniprot.org). Databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest.

As used herein, the term “modified protein” refers to a protein that contains one or more modifications from the wide-type protein. Examples of modifications include, but are not limited to, substitutions, deletions and insertions of one or more amino acid residues, and post transcriptional modifications such glycosylation.

As used herein, the terms “transfection,” “gene transfer”, and “gene delivery” are used interchangeably herein with reference to methods or systems for inserting foreign nucleic acids into host cells. Gene transfer can result in transient expression of non-integrated transferred DNA, extrachromosomal replication, and expression of transferred replicons (e.g., episomes), or integration of transferred genetic material into the genomic DNA of host cells. Various techniques are known to those of ordinary skill in the art to introduce one or more exogenous nucleic acid molecules, into suitable host cells, including chemical, electrical, and viral-mediated transfection procedures.

The term “host cell” as used herein generally refers to a cell (e.g., bacterial cell, yeast cell, insect cell, mammalian cell) which serves as a recipient for exogenously introduced nucleic acids or has been transfected with an exogenous nucleic acid. It should be understood that the progeny of a single parental cell may not necessarily be completely identical in morphology or in genomic or total DNA complement to the original parent, due to natural, accidental, or deliberate mutation. A host cell can be a cell that has been engineered to express a desired gene product (e.g., stably transformed to express one or more exogenous gene products). such a cell that stably expresses adenovirus Ela and Elb proteins (E1-expressing cell).

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

A nucleotide or amino acid residue in a first nucleic acid or protein “corresponds to” a residue in a second nucleic acid or protein if the two residues perform one or more corresponding functions and/or are located at corresponding positions in the first and second nucleic acids or proteins. Corresponding functions are typically the same, equivalent, or substantially equivalent functions, taking into account differences in the environments of the two nucleic acids or proteins as appropriate. Residues at corresponding positions typically align with each other when the sequences of the two nucleic acids or proteins are aligned to maximize identity (allowing the introduction of gaps) using a sequence alignment algorithm or computer program such as those referred to below (see “Identity”) and/or are located at positions such that when the 3-dimensional structures of the proteins is superimposed the residues overlap or occupy structurally equivalent positions and/or form the same, equivalent, or substantially equivalent intramolecular and/or intermolecular contacts or bonds (e.g., hydrogen bonds). The structures may be experimentally determined, e.g., by X-ray crystallography or NMR or predicted, e.g., using structure prediction or molecular modeling software. An alignment may be over the entire length of one or more of the aligned nucleic acid or polypeptide sequences or over at least one protein domain (or nucleotide sequence encoding a protein domain).

The term “expression cassette” is used herein with reference to a polynucleotide containing a gene of interest in operable linkage to regulatory elements (also termed “regulatory sequences”, “expression control elements”, or “expression control sequences”) mediating expression of a coding region therein in an mRNA transcript that can be translated into protein. The expression cassette may be contained or incorporated in a plasmid, virus vector, or may exist as a nucleic acid fragment.

An “expression vector” is a specialized vector that contains a gene or nucleic acid sequence with the necessary regulatory regions needed for expression in a host cell. Expression vectors include, for example, plasmids and viral vectors, such as adeno-associated virus (AAV) vectors, adenovirus vectors, lentivirus vectors, herpes virus vectors.

As used herein, the term “viral vector” refers to a recombinant polynucleotide vector comprising virally-derived nucleic acids containing sequences facilitating replication and expression of exogenously incorporated transgene sequences operatively linked to suitable control elements and one or more heterologous sequences (i.e., nucleic acid sequence not of viral origin).

The term “recombinant virus” is used herein with reference to a virus that has been genetically altered, e.g., by the addition or insertion of a heterologous nucleic acid construct into a virus particle.

The term “parvovirus” refers to a family of animal viruses that constitute the family Parvoviridae. They have linear, single-stranded DNA (ssDNA) genomes that typically contain two genes encoding for a replication initiator protein, called NS1, and the protein the viral capsid is made of. The coding portion of the genome is flanked by an inverted terminal repeat (ITR) at each end that form into hairpin loops that are important during replication. Parvovirus virions are small compared to most viruses, at 23-28 nanometers in diameter, and contain the genome enclosed in an icosahedral capsid that has a rugged surface. The parvovirus family contains three subfamilies and 126 species, including adeno-associated virus (AAV), bocavirus and protoparvovirus.

The term “parvovirus protein coding region” refers to the art-recognized region of a parvovirus genome which encodes non-structure proteins that are required to replicate the viral genome, such as NS1, and structure proteins for the viral capsid. The region need not include all of the wild-type genes but may be altered, e.g., by the insertion, deletion or substitution of nucleotides, so long as the non-structure proteins and structure proteins present provide for sufficient functions to allow production of the relevant recombinant parvovirus in a suitable host cell at a level of at least 25%, at least 40%, at least 50%, at least 60%, at least 70%, at least 80%, at least 90% or at least 95% of the level supported by a parvovirus protein coding region that encodes the corresponding wild-type non-structure and structure proteins.

The term “sequence encoding a recombinant parvovirus genome” refers to a nucleotide sequence that encodes sequences capable of producing a genome of a functional recombinant parvovirus in a suitable host cell. The sequence encoding a recombinant parvovirus genome typically comprises (1) an expression cassette comprising a sequence encoding a gene of interest (GOI), also referred to as a “transgene”, and a regulatory sequence operably linked to the sequence encoding the GOI, and (2) an ITR that flank at least one end of the expression cassette. In some embodiments, the sequence encoding a recombinant parvovirus genome comprises an ITR at each end of the expression cassette.

The term “sequence encoding a recombinant AAV genome” refers to a nucleotide sequence that encodes sequences capable of producing a genome of a functional recombinant AAV in a suitable host cell. The sequence encoding a recombinant AAV genome typically comprises (1) an expression cassette comprising a sequence encoding a GOI, also referred to as a “transgene”, and a regulatory sequence operably linked to the sequence encoding the GOI, and (2) an ITR that flank at least one end of the expression cassette. In some embodiments, the sequence encoding a recombinant AAV genome comprises an ITR at each end of the expression cassette.

The term “adeno-associated virus” (AAV) in the context of the present application includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, caprine AAV, porcine AAV, ovine AAV and any other AAV now known or later discovered serotypes and variants.

The terms “AAV cis construct” and “AAV vector” are used interchangeably herein with reference to a polynucleotide vector comprising one or more heterologous sequences (i.e., nucleic acid sequence not of AAV origin) that are flanked by at least one AAV ITR. Such AAV vectors can be replicated and packaged into infectious viral particles when present in a host cell that has been infected with a suitable helper virus (or that is expressing suitable helper functions) and that is expressing AAV Rep and Cap gene products (i.e. AAV Rep and Cap proteins). When an AAV vector is incorporated into a larger polynucleotide (e.g., in a chromosome or in another vector such as a plasmid used for cloning or transfection), then the AAV vector may be referred to as a “pro-vector” which can be “rescued” by replication and encapsidation in the presence of AAV packaging functions and suitable helper functions. An AAV vector can be in any of a number of forms, including, but not limited to, plasmids, linear artificial chromosomes, complexed with lipids, encapsulated within liposomes, and encapsidated in a viral particle, e.g., an AAV particle. An AAV vector can be packaged into an AAV virus capsid to generate a “recombinant adeno-associated viral particle (AAV particle).”

The “AAV cis construct” or “AAV vector” may be derived from any adeno-associated virus serotype, including without limitation, such as AAV-1, AAV-2, AAV-3, AAV-4, AAV-5, AAV-6, AAV-7, AAV-8, AAV-9, AAV-10 and AAV-11 and others described herein. AAV vectors can have one or more of the AAV wild-type genes deleted in whole or part, preferably the rep and/or Cap genes, but retain functional flanking ITR sequences. Functional ITR sequences are necessary for the rescue, replication, and packaging of the AAV virion. Thus, an AAV cis polynucleotide or AAV vector is defined herein to include at least those sequences required in cis for replication and packaging (e.g., functional ITRs) of the virus. The ITRs need not be the wild-type nucleotide sequences, and may be altered, e.g., by the insertion, deletion, or substitution of nucleotides, so long as the sequences provide for functional rescue, replication and packaging.

The term “AAV vector” also includes self-complementary adeno-associated virus (scAAV) vectors. A scAAV vector is a viral vector engineered from the naturally occurring AAV. scAAV is termed “self-complementary” because the coding region has been designed to form an intra-molecular double-stranded DNA template. A rate-limiting step during replication of the standard AAV genome involves the second-strand synthesis since the typical AAV genome is a single-stranded DNA template. However, this is not the case for scAAV genomes. Upon infection, rather than waiting for cell mediated synthesis of the second strand, the two complementary halves of scAAV will associate to form one double stranded DNA (dsDNA) unit that is ready for immediate replication and transcription.

The terms “recombinant AAV virion,” “rAAV virion,” and “rAAV virus particle” are used interchangeably herein with reference to an infectious, replication-defective virus particle composed of viral particle composed of at least one AAV capsid protein and an encapsidated rAAV vector genome comprising a heterologous nucleotide sequence of interest that is flanked on both sides by AAV ITRs. A rAAV virion is produced in a suitable host cell comprising an AAV vector, AAV helper functions, and accessory functions. A host cell containing these components is capable of encoding AAV polypeptides required for packaging the AAV vector (containing a recombinant nucleotide sequence of interest) into infectious recombinant virion particles for subsequent gene delivery.

The term “adeno-associated virus inverted terminal repeats” or “AAV ITRs” refers to the art-recognized regions found at each end of the AAV genome which function together in cis as origins of DNA replication and as packaging signals for the viral genome. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are well known in the art. As used herein, an “AAV ITR” need not have the wild-type nucleotide sequence depicted in the previously cited references, but may be altered, e.g., by the insertion, deletion, or substitution of nucleotides. Additionally, the AAV ITR may be derived from any of several AAV serotypes, including without limitation, AAV-1, AAV-2, AAV-3, AAV-4, AAV-5, AAV-6, AAV-7, AAV-8, AAV-9, AAV-10, AAV-11 etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

The term “Rep coding region” refers to the art-recognized region of a parvovirus that encodes a parvovirus Rep protein. The term also includes functional homologues thereof, such as the human herpesvirus 6 (HHV-6) rep gene which is also known to mediate AAV DNA replication and the rep genes in other parvoviruses such as bocavirus and protoparvovirus.

The term “AAV Rep coding region” refers to the art-recognized region of the AAV genome which encodes the replication proteins of the virus which are required to replicate the viral genome and to insert the viral genome into a host genome during latent infection. The Rep coding region, as used herein, can be derived from any viral serotype, such as the AAV serotypes described above.

The region need not include all of the wild-type genes but may be altered, e.g., by the insertion, deletion or substitution of nucleotides, so long as the altered rep genes present provide for sufficient integration functions when expressed in a suitable host cell. An altered Rep gene will be considered to encode a “functional variant” of a wild-type Rep protein, if the function variant supports rAAV production at a level at least 25%, at least 40%, at least 50%, at least 70%, at least 80%, at least 90% or at least 95% of the level supported by the wild type rep gene product.

The term “long forms of Rep” refers to the Rep 78 and Rep 68 gene products of the AAV Rep coding region, including functional homologues thereof. The long forms of Rep are normally expressed under the direction of the AAV p5 promoter.

The phrase “short forms of Rep” refers to the Rep 52 and Rep 40 gene products of the AAV Rep coding region, including functional homologues thereof. The short forms of Rep are expressed under the direction of the AAV p19 promoter.

The term “Cap coding region” refers to the art-recognized region of the parvoviruse genome which encodes the coat proteins of the virus which are required for packaging the viral genome.

The term “AAV Cap coding region” refers to the art-recognized region of the AAV genome which encodes the coat proteins of the virus (e.g., VP1, VP2 and VP3) which are required for packaging the viral genome. The AAV Cap coding region, as used herein, can be derived from any AAV serotype, as described above. The region need not include all of the wild-type Cap genes but may be altered, e.g., by the insertion, deletion or substitution of nucleotides, so long as the altered Cap genes provide for sufficient packaging functions when present in a host cell along with an AAV vector. An altered Cap gene will be considered to encode a “functional variant” of a wild-type Cap protein, if the function variant supports rAAV production at a level at least 25%, at least 40%, at least 50%, at least 70%, at least 80%, at least 90% or at least 95% of the level supported by the wild type Cap gene product.

The term “AAV helper functions” refers to AAV-derived coding sequences that can be expressed to provide AAV gene products that, in turn, function in trans for productive AAV replication. Thus, AAV helper functions include the Rep and Cap regions. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

The term “AAV trans construct” or “AAV helper construct” refers to a nucleic acid molecule that includes nucleotide sequences providing AAV functions deleted from an AAV vector which is to be used to produce a transducing vector for delivery of a nucleotide sequence of interest. AAV trans constructs are commonly used to provide transient expression of AAV Rep and/or Cap genes to complement missing AAV functions that are necessary for lytic AAV replication; however, trans constructs lack AAV ITRs and can neither replicate nor package themselves. AAV trans constructs can be in the form of a plasmid, mini-circle, phage, transposon, cosmid, virus, or virion. A number of AAV trans constructs have been described, such as the commonly used plasmids pAAV/Ad and pIM29+45 which encode both Rep and Cap expression products. See, e.g., Samulski et al. (1989) J. Virology 63, 3822-3828; McCarty et al. (1991) J. Virology 65, 2936-2945. A number of other vectors have been described which encode Rep and/or Cap expression products. See, e.g., U.S. Pat. No. 5,139,941.

The term “adenovirus” as used herein, has the same meaning as an adenovirus vector, and refers to a member of the family Adenoviridae. The Adenoviridae includes all animal adenoviruses of the genus Mastadenovirus. In particular, human adenoviruses include the A-F subgenera and the individual serotypes thereof. The A-F subgenera includes human adenovirus types 1, 2, 3, 4, 4a, 5, 6, 7, 8, 9, 10, 11 (Ad11A and Ad11P), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48 and 51.

The term “accessory functions” refers to non-AAV derived viral and/or cellular functions upon which AAV is dependent for its replication. Thus, the term embraces DNAs, RNAs and protein that are required for AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

Adenovirus-derived accessory functions have been widely studied, and a number of adenovirus genes involved in accessory functions have been identified and partially characterized. Specifically, early adenoviral E1A, E1B 55K, E2A, E4, and VA RNA gene regions are thought to participate in the accessory process.

A “functional homolog” or a “functional equivalent” of a given adenoviral nucleotide region includes similar regions derived from a heterologous adenovirus serotype, nucleotide regions derived from another virus or from a cellular source, and recombinantly produced or chemically synthesized polynucleotides which function in a manner similar to the reference nucleotide region to achieve a desired result. Thus, a functional homolog of an adenoviral VA RNA gene region or an adenoviral E2A gene region encompasses derivatives and analogues of such gene regions—including any single or multiple nucleotide base additions, substitutions and/or deletions occurring within the regions, so long as the homologue retains the ability to provide its inherent accessory function to support AAV virion production at levels detectable above background.

The term “percent identical” is used herein with reference to comparisons among nucleic acid or amino acid sequences. Nucleic acid and amino acid sequences are often compared using computer programs in e.g. the National Library of Medicine BLAST alignment program.

### II. Compositions and Kits

One aspect of the present application relates to an expression construct for production of recombinant parvovirus, such as an rAAV. The expression construct of the present application encodes and is capable of expressing adenovirus proteins that provides accessory functions for production of recombinant parvovirus in a host cell.

In some embodiments, the expression construct of the present application is capable of providing adenovirus helper functions, such as adenovirus E4, E2, VA RNA and/or E1 functions, and can be used in a triple-plasmid system for recombinant parvovirus production.

In some embodiments, the expression construct of the present application comprises (1) sequences encoding adenovirus genes that provide adenovirus helper functions, and (2) a sequence encoding parvovirus non-structure and structure proteins, such as AAV Rep and Cap proteins, or a sequence encoding a recombinant parvovirus genome, and can be used in a dual-plasmid system for recombinant parvovirus production.

In some embodiments, the expression construct of the present application comprises (1) sequences encoding adenovirus genes that provide adenovirus helper functions, (2) a sequence encoding parvovirus non-structure and structure proteins, such as AAV Rep and Cap proteins, and (3) a sequence encoding a recombinant parvovirus genome, and can be used in a single-plasmid system for production of recombinant pravovirus. Table 1 shows some exemplary expression constructs for the production of rAAV.

TABLE 1
Exemplary expression constructs for AAV production.

| | | E4orf6 | | | | | |
|---|---|---|---|---|---|---|---|
| | | and/or | | | | | |
| | E2A | E4orf6/7 | VA RNA | Rep | Cap | AAV-GOI | Size Limit |
| Mini-pHelper | Yes | Yes | Yes | No | No | No | 2-11 kb |
| Mini -pHelper- | Yes | Yes | Yes | Yes | Yes | No | 6-15 kb |
| Rep-Cap | | | | | | | |
| Mini-pHelper-GOI | Yes | Yes | Yes | Yes | Yes | Yes | 3-16 kb |
| pAAVone-AAVs- | Yes | Yes | Yes | Yes | Yes | Yes | 7-20 kb |
| GOI (Mini-pHelper- | | | | | | | |
| Rep-Cap-GOI) | | | | | | | |
| Mini-pHelper-Rep | Yes | Yes | Yes | Yes | No | No | 4-13 kb |

In some embodiments, the expression construct of the present application also encodes functional adenovirus Ela and/or E1b proteins. In some embodiments, the expression construct of the present application does not encode a functional adenovirus Ela and/or a E1b proteins.

Expression Constructs for Triple-Plasmid Parvovirus Production System

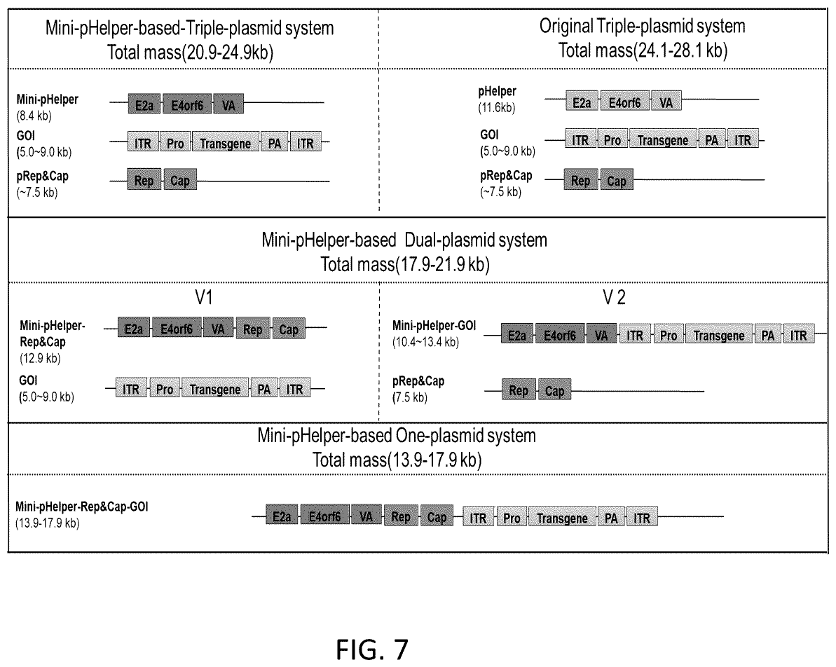

In some embodiments, the expression construct of the preset application is designed to be used in a triple-plasmid parvovirus production system. FIG. 7 shows an exemplary construct for a triple-plasmid AAV production system. The expression construct comprises a modified adenovirus E4 coding region or a modified adenovirus E2a coding region and is capable of expressing one or more adenovirus E4 proteins, or an adenovirus E2a protein, that are required for AAV production in a host cell.

In some embodiments, the expression construct comprises both a modified adenovirus E4 coding region and a modified adenovirus E2a coding region, and is capable of expressing one or more adenovirus E4 proteins and an adenovirus E2a protein to provide the necessary adenovirus helper function for the production of recombinant parvovirus in a suitable host cell.

In some embodiments, the expression construct comprises a modified adenovirus E4 coding region, a modified adenovirus E2a coding region, and a sequence encoding one or more adenovirus-associated RNAs (VA RNAs), and is capable of expressing the encoded adenovirus proteins and RNAs to provide the necessary adenovirus helper function for the production of recombinant parvovirus in a suitable host cell. In some embodiments, the expression construct does not encode a functional adenovirus Ela and/or E1b protein, and the host cells provide the Ela and/or E1b helper function.

In some embodiments, the expression construct comprises a modified adenovirus E4 coding region, a modified adenovirus E2a coding region, a sequence encoding one or more adenovirus-associated RNAs (VA RNAs), an AAV Rep coding region, and is capable of expressing the encoded adenovirus proteins, VA RNAs and the AAV Rep protein for the production of recombinant AAV in a suitable host cell.

Modified Adenovirus E4 Coding Region

The modified adenovirus E4 coding region encodes one or more functional E4 proteins capable of supporting a parvovirus replication in a host cell. In some embodiments, the one or more functional E4 proteins comprise only adenovirus E4 open reading frames 6/7 (E4orf6/7). In some embodiments, the one or more functional E4 proteins comprise adenovirus E4 open reading frames 6 (E4orf6) and E4orf6/7. In some embodiments, the one or more functional E4 proteins comprise a polypeptide sequence encoded by all or a significant portion of E4orfs6/7 and does not comprise polypeptide sequences encoded by all or a portion of E4orfs 1-4, E4orf6 and E4 34K protein. In some embodiments, the one or more comprise (1) a polypeptide sequence encoded by all or a significant portion of E4orf6, and (2) a polypeptide sequence encoded functional E4 proteins by all or a significant portion of E4orf6/7, wherein the one or more functional E4 proteins do not comprise polypeptide sequences encoded by all or a portion of E4orfs 1˜4 and E4 34K protein.

In some embodiments, the modified adenovirus E4 coding region comprises one or more deletions. In some embodiments, the modified adenovirus E4 coding region comprises a partial or complete deletion of E4orf6/7 intron 1. In some embodiments, the modified adenovirus E4 coding region comprises (1) a partial or complete coding sequence of E4orf6, (2) a partial or complete coding sequence of E4orf6/7, and (3) a partial or complete deletion of E4orf6/7 intron 1.

In some embodiments, the modified adenovirus E4 coding region comprises a partial deletion of E4orf6/7 intron 1, wherein up to 10%, 20, 30%, 40%, 50%, 60%, 70%, 80%, 90% or 95% of the E4orf6/7 intron 1 sequence is deleted.

In some embodiments, the modified adenovirus E4 coding region comprises a partial or complete deletion of E4orf6/7 intron 2. In some embodiments, the modified adenovirus E4 coding region comprises a partial deletion of E4orf6/7 intron 2, wherein up to 10%, 20, 30%, 40%, 50%, 60%, 70%, 80%, 90% or 95% of the E4orf6/7 intron 2 sequence is deleted.

In some embodiments, the modified adenovirus E4 coding region comprises a partial or complete deletion of E4orf6/7 intron 1 and a partial or complete deletion of E4orf6/7 intron 2.

In some embodiments, the modified adenovirus E4 coding region has a total size of 400-500, 400-600, 400-800, 400-1000, 400-1200, 400-1500, 400-2000, 500-600, 500-800, 500-1000, 500-1200, 500-1500, or 500-2000 bp and encodes a functional E4 protein that is capable of providing the required E4 functions for parvovirus production in a non-E4 expressing host cell.

In some embodiments, the modified adenovirus E4 coding region comprises a sequence corresponding to nucleotide sequence 32645-35835 of the Ad2 genome with (a) a partial or complete deletion of E4orf6/7 intron 1, (b) a partial or complete deletion of E4orf6/7 intron 2, or both (a) and (b).

In some embodiments, the modified adenovirus E4 coding region comprises a sequence corresponding to nucleotide sequence 32645-35835 of the Ad2 genome with a deletion of the complete sequence of E4orf6/7 intron 2 (711 bp, SEQ ID NO:2) and a deletion of the complete sequence of E4orf6/7 intron 1 (1275 bp, SEQ ID NO:1). In some embodiments, the modified adenovirus E4 coding region comprises a codon optimized nucleotide sequence (SEQ ID NO:7) encoding adenovirus E4orf6/7 protein (SEQ ID NO:8).

In some embodiments, the modified adenovirus E4 coding region comprises a sequence corresponding to nucleotide sequence 32645-35835 of the Ad2 genome with a deletion of the complete sequence of E4orf6/7 intron 1 (1275 bp, SEQ ID NO:1). In some embodiments, the modified adenovirus E4 coding region comprises a nucleotide sequence (SEQ ID NO:24) encoding adenovirus E4orf6 protein (SEQ ID NO:25) or a codon optimized nucleotide sequence (SEQ ID NO:25) encoding adenovirus E4orf6 protein (SEQ ID NO:26). a codon optimized nucleotide sequence (SEQ ID NO:7) encoding adenovirus E4orf6/7 protein (SEQ ID NO:8).

Modified Adenovirus E2a Coding Region

In some embodiments, the modified adenovirus E2a coding region comprises (a) a partial or complete deletion of early-E2a or later-E2a intron 1, (b) a partial or complete deletion of E2a intron 2, or both (a) and (b).

In some embodiments, the modified adenovirus E2a coding region comprises a partial deletion of later-E2a intron 1 and/or later-E2a intron 2, wherein up to 10%, 20, 30%, 40%, 50%, 60%, 70%, 80%, 90% or 95% of the later-E2a intron 1 and/or later-E2a intron 2 is deleted.

In some embodiments, the modified adenovirus E2a coding region has a total size of 1600-3500, 1600-3000, 1600-2500, 1600-2000, 1600-1800 or 1600-1700 bp and encodes a functional E2a protein that is capable of providing the required E2a functions for parvovirus production in a non-E2a expressing host cell.

In some embodiments, the modified adenovirus E2a coding region comprises a sequence corresponding to nucleotide sequence 22233-27575 of the Ad2 genome with (a) a partial or complete deletion of early-E2a or later-E2a intron 1, (b) a partial or complete deletion of E2a intron 2, or both (a) and (b). In some embodiments, the modified adenovirus E2a coding region comprises a sequence corresponding to nucleotide sequence 22233-27575 of the Ad2 genome with a 1897 bp deletion (SEQ ID NO:3) that encompasses the complete sequence of later-E2a intron 1 and later-E2a intron 2. In some embodiments, the modified adenovirus E2a coding region comprises a partial deletion of later-E2a intron 1 and/or later-E2a intron 2, wherein up to 10%, 20, 30%, 40%, 50%, 60%, 70%, 80%, 90% or 95% of the SEQ ID NO:3 is deleted.

In some embodiments, the modified adenovirus E2a coding region comprises (a) a partial or complete deletion of later a sequence encoding a codon optimized adenovirus E2a protein E2a intron 1, (b) a partial or complete deletion of later E2a intron 2, and (c) a codon modified nucleotide sequence encoding an adenovirus E2a protein.

Adenovirus VA RNA Coding Region

In some embodiments, the expression construct further comprises an adenovirus VA RNA coding region that encodes one or more adenovirus VA RNAs. In some embodiments, the expression construct comprises an adenovirus VA RNA coding region that encodes adenovirus VAI RNA and/or VAII RNA. In some embodiments, the VA RNA coding region encodes functional VA RNA I and/or VA RNAII that are capable of providing the required VA RNA functions for parvovirus production in a non-VA RNA expressing host cell. In some embodiments, the expression construct comprises SEQ ID NO:16.

As used herein and thereafter, a “functional” adenovirus E4 protein, adenovirus E2a protein or adenovirus VA RNA I or VA RNA II, is able to facilitate production of a recombinant parvovirus, such as bocavirus. It is within the abilities of the skilled person to determine whether a given VA RNA I and II, E2A protein or E4 protein is functional. In an embodiment, an adenovirus E4 protein, adenovirus E2a protein or adenovirus VA RNA I or VA RNA II will be considered to be “functional” if it supports parvovirus production at a level at least 25%, at least 40%, at least 50%, at least 70%, at least 80%, at least 90% or at least 95% of the level supported by the corresponding wild type protein or RNA in the same tri-plasmid, dual-plasmid or single-plasmid system.

Adenoviruses and their nucleic acid fragments for use in the compositions and methods of the present application include all human adenoviruses of the Adenoviridae family, which include members of the Mastadenovirus genera. To date, over fifty-one human serotypes of adenoviruses have been identified. The adenovirus may be of serogroups A, B, C, D, E, or F. The human adenovirus may be a serotype 1 (Ad 1), serotype 2 (Ad2), serotype 3 (Ad3), serotype 4 (Ad4), serotype 5 (Ad5), serotype 6 (Ad6), serotype 7 (Ad7), serotype 8 (Ad8), serotype 9 (Ad9), serotype 10 (Ad10), serotype 11 (Ad11), serotype 12 (Ad12), serotype 13 (Ad13), serotype 14 (Ad14), serotype 15 (Ad15), serotype 16 (Ad16), serotype 17 (Ad17), serotype 18 (Ad18), serotype 19 (Ad19), serotype 19a (Ad19a), serotype 19p (Ad19p), serotype 20 (Ad20), serotype 21 (Ad21), serotype 22 (Ad22), serotype 23 (Ad23), serotype 24 (Ad24), serotype 25 (Ad25), serotype 26 (Ad26), serotype 27 (Ad27), serotype 28 (Ad28), serotype 29 (Ad29), serotype 30 (Ad30), serotype 31 (Ad31), serotype 32 (Ad32), serotype 33 (Ad33), serotype 34 (Ad34), serotype 35 (Ad35), serotype 36 (Ad36), serotype 37 (Ad37), serotype 38 (Ad38), serotype 39 (Ad39), serotype 40 (Ad40), serotype 41 (Ad41), serotype 42 (Ad42), serotype 43 (Ad43), serotype 44 (Ad44), serotype 45 (Ad45), serotype 46 (Ad46), serotype 47 (Ad47), serotype 48 (Ad48), serotype 49 (Ad49), serotype 50 (Ad50), serotype 51 (Ad51), or combinations thereof, but are not limited to these examples. In certain embodiments, the adenovirus is serotype 5 (Ad5).

Regulatory Elements

In some embodiments, the expression construct of the present application comprises one or more regulatory elements that control expression of the encoded adenovirus proteins and/or RNAs. Exemplary promoters include, for example, viral promoters, mammalian promoters, composite promoters, cell type- or tissue-specific promoters, and inducible/repressible promoters.

Viral promoters include, for example, the SV40 early promoter, mouse mammary tumor virus LTR promoter; adenovirus major late promoter (Ad MLP); adenovirus E4 promoter, adenovirus e2a promoter, herpes simplex virus (HSV) promoter, cytomegalovirus (CMV) promoter, such as the CMV immediate early promoter region (CMV-IE), SFFV promoter, and Rous Sarcoma Virus (RSV) promoter.

Mammalian promoters include, for example, those for expressing EFla, ubiquitin (e.g., ubiquitin B or C), globin, actin, phosphoglycerate kinase (PGK), NSE (neuronal specific enolase), synapsin or NeuN, as well as composite promoters, such as the CAG promoter (combination of the CMV early enhancer element and chicken beta-actin promoter).

The promoters can be of human origin or from other species, including mice. In some embodiments a human promoter may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase I promoter, e.g., a promoter for transcription of ribosomal RNA (other than 5S rRNA) or a functional variant thereof may be used. In some embodiments, a promoter that ordinarily directs transcription by a eukaryotic RNA polymerase III (a “pol III promoter”), e.g., (a U6, H1, 7SK or tRNA promoter or a functional variant thereof) may be used.

In some embodiments, the modified adenovirus E4 coding region in the expression construct of the present application is operably linked to a regulatory element, such as a promoter, an enhancer or both. In some embodiments, the modified adenovirus E4 coding region is operably linked to an adenovirus E4 promoter. In some embodiments, the modified adenovirus E4 coding region is operably linked to an adenovirus E4 promoter comprising SEQ ID NO:22.

In some embodiments, the modified adenovirus E2a coding region in the expression construct of the present application is operably linked to a regulatory element, such as a promoter, an enhancer or both. In some embodiments, the modified adenovirus E2a coding region is operably linked to an adenovirus E2a promoter. In some embodiments, the modified adenovirus E2a coding region is operably linked to an adenovirus E2a promoter comprising SEQ ID NO:23.

Codon Optimized Coding Sequences

In some embodiments, the expression construct of the present application includes one or more polynucleotides encoding one or more codon optimized proteins for expression in e.g., human, mammalian or primate cells, such as HuH7, HEK293T or CHO cells. The polynucleotides encoding the proteins of the present application may be codon optimized to improve the activity, stability or expression in host cells without changing the encoded amino acid sequence. Codon optimization replaces codons present in a polynucleotide sequence with preferred codons encoding the same amino acid, for example, codons preferred for mammalian expression. Thus, the amino acid sequence is not altered during the process. Codon optimization can be performed using gene optimization software. The codon optimized nucleotide sequence is translated and aligned to the original protein sequence to ensure that no changes were made to the amino acid sequence. Methods of codon optimization are known in the art and are described, for example, in U.S. Application Publication No. 2008/0194511 and U.S. Pat. No. 6,114,148.

In some embodiments, the expression construct of the present application comprises (a) a modified adenovirus E4 coding region comprising a codon-modified E4 coding sequence or (b) a modified adenovirus E2a coding region comprising a codon-modified E2a coding sequence.

In some embodiments, the expression construct of the present application comprises (a) a modified adenovirus E4 coding region comprising a codon-modified E4 coding sequence and (b) a modified adenovirus E2a coding region comprising a codon-modified E2a coding sequence.

In some embodiments, the expression construct of the present application comprises a modified adenovirus E4 coding region comprising a codon modified nucleotide sequence (SEQ ID NO:7) encoding E4orf6/7. In some embodiments, the expression construct of the present application comprises a modified adenovirus E2a coding region comprising a codon modified nucleotide sequence (SEQ ID NO:4) encoding E2a.

In some embodiments, the expression construct of the present application comprises a modified adenovirus E4 coding region comprising a codon modified nucleotide sequence (SEQ ID NO:25) encoding E4orf6.

In some embodiments, the expression construct of the present application comprises a modified adenovirus E4 coding region comprising a codon modified nucleotide sequence (SEQ ID NO:25) encoding E4orf6 and a codon modified nucleotide sequence (SEQ ID NO:7) encoding E4orf6/7.

Additional Elements

In some embodiments, the expression construct of the present application further comprises a sequence that encodes adenovirus Ela and E1b proteins.

In some embodiments, the expression construct further comprises one or more additional elements, such as plasmid replication origin sequences and selection markers. Examples of selection marks include, but are not limited to, ampicillin resistant genes, neomycine resistant genes and kanamycin resistant genes.

In some embodiments, the expression construct designed for triple-plasmid pravovirus production system has a size of less than 13 kb, 12 kb, 11 kb, 10 kb, 9 kb, 8 kb, 7 kb, 6, kb, 5 kb, 4 kb, 3 kb or 2 kb. In some embodiments, the expression construct has a size in the range of 2-11 kb, 2-4 kb, 2-6 kb, 2-8 kb, 2-10 kb, 4-6 kb, 4-8 kb, 4-10 kb, 4-11 kb, 6-8 kb, 6-10 kb, 6-11 kb, 8-10 kb, 8-11 kb or 10-11 kb.

In some embodiments, the expression construct designed for triple-plasmid pravovirus production system comprises a modified adenovirus E4 coding region, a modified adenovirus E2a coding region, a sequence encoding one or more adenovirus-associated RNAs (VA RNAs), a sequence encoding an AAV Rep protein, and has a size of less than 13 kb, 12 kb, 11 kb, 10 kb, 9 kb, 8 kb, 7 kb, 6, kb, 5 kb, or 4 kb. In some embodiments, the expression construct has a size in the range of 4-13 kb, 4-11 kb, 4-9 kb, 4-7 kb, 7-13 kb, 7-11 kb, 7-9 kb, 9-13 kb, 9-11 kb, or 11-13 kb.

Preferred Embodiments of Expression Constructs for Triple-Plasmid Parvovirus Production System

In some embodiments, the expression construct is a plasmid for triple-plasmid AAV production. The plasmid comprises a modified adenovirus E4 coding region comprising coding sequences for E4orf6 and E4orf6/7, a modified adenovirus E2a coding region comprising coding sequence for E2a, and a sequence that encodes adenovirus VAI RNA coding region, wherein the plasmid has a size in the range of 6-7 kb, 6-8 kb, 6-9 kb, 7-8 kb, 7-9 kb and 8-9 kb. In some embodiments, the plasmid has a size of 7-9 kb.

In some embodiments, the expression construct is a plasmid for triple-plasmid AAV production. The plasmid comprises a modified adenovirus E4 coding region comprising SEQ ID NO:17 or SEQ ID NO:18, a modified adenovirus E2a coding region comprising SEQ ID NO:4, a sequence that encodes adenovirus VAI RNA coding region comprising SEQ ID NO:16, and a regulatory region comprising SEQ ID NO:19, wherein the plasmid has a size in the range of 6-7 kb, 6-8 kb, 6-9 kb, 7-8 kb, 7-9 kb and 8-9 kb. In some embodiments, the plasmid has a size of 7-9 kb.

Expression Constructs for Dual-Plasmid Parvovirus Production System

In some embodiments, the expression construct of the preset application is designed to be used in a dual-plasmid parvovirus production system. FIG. 7 shows two exemplary dual-plasmid systems for AAV production.

Constructs Encoding Adenovirus Proteins and Parvovirus Proteins

In some embodiments, the expression constructs for the dual-plasmid parvovirus production comprise, in addition to one or more of the elements described in the expression constructs for triple-plasmid parvovirus production system, a parvovirus protein coding region. In some embodiments, the parvovirus coding region comprises a sequence encompassing an AAV Rep coding region and a sequence encompassing an AAV Cap coding region.

In some embodiments, the expression construct for dual-plasmid parvovirus production is constructed for AAV production and comprises (1) a modified adenovirus E4 coding region that encodes (a) E4orf6/7 or (b) both E4orf6 and E4orf6/7, (2) a modified adenovirus E2a coding region that encodes an E2a protein, (3) an adenovirus VA RNA coding region, (4) an AAV Rep coding region, (5) an AAV Cap coding region, and (6) one or more regulatory elements that allow expression of the E4orf6/7 or both E4orf6 and E4orf6/7; the E2a protein, the adenovirus VA RNAs, the AAV Rep protein, and the AAV Cap protein in a host cell. As shown in FIG. 7, such an expression construct may be used in combination with another expression vector encoding a recombinant AAV genome (e.g., a transgene expression cassette flanked by at least one ITR at one side) for production of the recombinant AAV in a host cell. In some embodiments, the AAV Rep coding region is operably linked to one or more regulatory elements, such as an AAV P5 promoter or AAV P5I promoter. In some embodiments, the AAV Rep coding region is operably linked to one or more regulatory elements, such as an AAV P19 promoter. In some embodiments, the regulatory element comprises a P5I promoter (SEQ ID NO:15). In some embodiments, the expression construct for dual-plasmid AAV production further comprises a sequence that encodes adenovirus Ela and/or E1b proteins. In some embodiments, the expression construct for dual-plasmid AAV production does not comprise a sequence that encodes adenovirus Ela or E1b protein. In some embodiments, the expression construct has a size of less than 15 kb, 14 kb, 13 kb, 12 kb, 11 kb, 10 kb, 9 kb, 8 kb, or 7 kb. In some embodiments, the expression construct has a size in the range of 6-15 kb, 6-13 kb, 6-11 kb, 6-9 kb, 9-15 kb, 9-13 kb, 9-11 kb, 11-15 kb, 11-13 kb or 13-15 kb.

Construct Encoding Adenovirus Proteins and Recombinant Parvovirus Genome

In some embodiments, the expression construct for dual-plasmid parvovirus production system comprises (1) a modified adenovirus E4 coding region that encodes (a) E4orf6/7 or (b) both E4orf6 and E4orf6/7, (2) a modified adenovirus E2a coding region that encodes an E2a protein, (3) a sequence that encodes an adenovirus VA RNA, (4) a sequence encoding a recombinant parvovirus genome (e.g., a transgene expression cassette flanked by at least one ITR on one side), and (5) one or more regulatory elements that allow expression of the E4 open reading frame 6/7; the E2a protein, and the adenovirus VA RNA in a host cell.

In some embodiments, the expression construct designed for dual-plasmid AAV production system comprise (1) a modified adenovirus E4 coding region that encodes (a) E4orf6/7 or (b) both E4orf6 and E4orf6/7, (2) a modified adenovirus E2a coding region that encodes an E2a protein, (3) a sequence that encodes an adenovirus VA RNA, (4) a sequence encoding a recombinant AAV genome (e.g., a transgene expression cassette flanked by two ITRs), (5) one or more regulatory elements that allow expression of the E4 open reading frame 6/7; the E2a protein, and the adenovirus VA RNA in a host cell. As shown in FIG. 7, such an expression construct may be used in combination with another expression vector encoding the AAV Rep and Cap proteins for production of the recombinant AAV in a host cell. In some embodiments, the expression construct for dual-plasmid AAV production further comprises a sequence that encodes adenovirus Ela and E1b proteins. In some embodiments, the expression construct has a size of less than 16 kb, 15 kb, 14 kb, 13 kb, 12 kb, 11 kb, 10 kb, 9 kb, 8 kb, 7 kb, 6 kb, 5 kb, or 4 kb. In some embodiments, the expression construct has a size in the range of 3-16 kb, 3-14 kb, 3-12 kb, 3-10 kb, 3-8 kb, 3-6 kb, 6-16 kb, 6-14 kb, 6-12 kb, 6-10 kb, 6-8 kb, 8-16 kb, 8-14 kb, 8-12 kb, 8-10 kb, 10-16 kb, 10-14 kb, 10-12 kb, 12-16 kb, 12-14 kb, or 14-16 kb.

Preferred Embodiments of Expression Constructs for Dual-Plasmid AAV Production System

In some embodiments, the expression construct for dual-plasmid parvovirus production is a plasmid for AAV production. The plasmid comprises a modified adenovirus E4 coding region comprising coding sequences for E4orf6 and E4orf6/7, a modified adenovirus E2a coding region comprising coding sequence for E2a, a sequence that encodes adenovirus VAI RNA coding region, a sequence that encodes an AAV Rep protein, and a sequence that encodes an AAV Cap protein, wherein the plasmid has a size in the range of 6-7 kb, 6-8 kb, 6-9 kb, 7-8 kb, 7-9 kb and 8-9 kb. In some embodiments, the plasmid has a size of 7-9 kb.

In some embodiments, the expression construct for dual-plasmid parvovirus production system is a plasmid for AAV production. The plasmid comprises a modified adenovirus E4 coding region comprising SEQ ID NO:17 or SEQ ID NO:18, a modified adenovirus E2a coding region comprising SEQ ID NO:4, a sequence that encodes adenovirus VAI RNA coding region comprising SEQ ID NO:16, a regulatory region comprising SEQ ID NO:19, a sequence that comprise SEQ ID NO:20 and encodes an AAV Rep protein, and a sequence that comprising SEQ ID NO:21 and encodes an AAV Cap protein, wherein the plasmid has a size in the range of 12-14 kb.

In some embodiments, the expression construct for dual-plasmid parvovirus production system is a plasmid for AAV production. The plasmid comprises a modified adenovirus E4 coding region comprising SEQ ID NO:17 or SEQ ID NO:18, a modified adenovirus E2a coding region comprising SEQ ID NO:4, a sequence that encodes adenovirus VAI RNA coding region comprising SEQ ID NO:16, a regulatory region comprising SEQ ID NO:19, a sequence that encodes a recombinant AAV genome, wherein the recombinant AAV genome comprises transgene expression cassette comprising a transgene of interest operably linked to one or more regulatory elements, and two AAV ITRs flanking the transgene expression cassette, wherein the plasmid has a size in the range of 10-14 kb.

Expression Construct for One-Plasmid Parvovirus Production System

In some embodiments, the expression construct of the preset application is designed to be used in a single-plasmid parvovirus production system. The expression construct contains any or all of the foregoing modified adenovirus coding regions, a parvovirus protein coding region, as well as sequences encoding a recombinant parvovirus genome that comprises an expression cassette having a GOI operably liked to a regulatory element, and at least one ITR at one end of the expression cassette. The expression construct allows for production of a recombinant parvovirus by transfecting a host cell with a single plasmid. In some embodiments, the expression construct does not provide adenovirus E1a and E1b function and needs to be used for parvovirus production in host cells that provide endogenous expression of adenovirus E1a and E1b proteins. In some embodiments, the expression construct also provides adenovirus Ela and E1b function and can be used for parvovirus production in host cells that do not provide endogenous expression of adenovirus E1a and E1b proteins.

FIG. 7 provides an exemplary embodiment for an expression construct for a single-plasmid AAV production system. In some embodiments the expression construct Comprises (1) a modified adenovirus E4 coding region that encodes (a) E4orf6/7 or (b) E4orf6 and E4orf6/7, (2) a modified adenovirus E2a coding region that encodes an E2a protein, (3) an adenovirus VA RNA coding region, (4) an AAV Rep coding region, (5) an AAV Cap coding region, (6) a sequence encoding a recombinant AAV genome, comprising an expression cassette having a nucleotide sequence encoding a transgene and a regulatory element operably linked to the nucleotide sequence, and at least one ITR at one end of the expression cassette, and (7) one or more regulatory elements that allow expression of the E4orf6 and/or E4orf6/7; the E2a protein, the adenovirus VA RNA, the AAV Rep protein, and the AAV Cap protein in a host cell. In some embodiments, the expression cassette in the recombinant AAV genome is flanked by an AAV ITR on each end.

In some embodiments, the expression construct of the preset application comprises (1) a modified adenovirus E4 coding region that encodes (a) E4orf6/7 or (b) E4orf6 and E4orf6/7, (2) a modified adenovirus E2a coding region that encodes an E2a protein, (3) an adenovirus VA RNA coding region, (4) a parvovirus protein coding region, (5) a recombinant parvovirus cloning region, and (6) one or more regulatory elements that allow expression of the E4orf6 and/or E4orf6/7; the E2a protein, the adenovirus VA RNA, and parvovirus proteins in a host cell.

In some embodiments, the recombinant parvovirus cloning region comprises a cloning site for insertion of a recombinant parvovirus sequence comprising (1) an expression cassette comprising a gene of interest (GOI) operably linked to a regulatory element and (2) at least one ITR at one side of the expression cassette. In some embodiments, the recombinant parvovirus sequence comprises two ITRs, one at each end of the expression cassette.

In some embodiments, a recombinant parvovirus cloning region comprises (1) a cloning site for insertion of a sequence comprising an expression cassette comprising a GOI operably linked to a regulatory element, and (2) at least one ITR at one side of the cloning site. In some embodiments, the recombinant parvovirus sequence comprises two ITRs, one at each side of the cloning site.

In some embodiments, a recombinant parvovirus cloning region comprises (1) an expression cassette comprising a cloning site for insertion of a GOI and a regulatory element in the proximity of the cloning site and is capable of being operably linked to the GOI when the GOI is inserted at the cloning site, and (2) at least one ITR at one side of the expression cassette. In some embodiments, the recombinant parvovirus sequence comprises two ITRs, one at each side of the expression cassette.

In some embodiments, the modified adenovirus E4 coding region encodes E4orf6 and E4orf6/7 with a partial or complete deletion of E4orf6/7 intron 1. In some embodiments, the modified adenovirus E4 coding region encodes E4orf6/7 with a partial or complete deletion of E4orf6/7 intron 2. In some embodiments, the modified adenovirus E4 coding region encodes E4orf6/7 with a partial or complete deletion of both E4orf6/7 intron 1 and E4orf6/7 intron 2.

In some embodiments, the modified adenovirus E4 coding region has a total size of 400-500, 400-600, 400-800, 400-1000, 400-1200, 400-1500, 400-2000, 500-600, 500-800, 500-1000, 500-1200, 500-1500, or 500-2000 bp and encodes a functional E4 protein that is capable of providing the required E4 functions for parvovirus production in a non-E4 expressing host cell.

In some embodiments, the modified adenovirus E4 coding region comprises a sequence corresponding to nucleotide sequence 32645-35835 of the Ad2 genome with (a) a partial or complete deletion of E4orf6/7 intron 1, (b) a partial or complete deletion of E4orf6/7 intron 2, or both (a) and (b). In some embodiments, the modified adenovirus E4 coding region comprises a sequence corresponding to nucleotide sequence 32645-35835 of the Ad2 genome with a partial or complete deletion of E4orf6/7 intron 1.

In some embodiments, the modified adenovirus E4 coding region comprises a sequence corresponding to nucleotide sequence 32645-35835 of the Ad2 genome with a deletion of the complete sequence of E4orf6/7 intron 2 (711 bp, SEQ ID NO:2) and a deletion of the complete sequence of E4orf6/7 intron 1 (1275 bp, SEQ ID NO:1).

In some embodiments, the modified adenovirus E4 coding region comprises a sequence corresponding to nucleotide sequence 32645-35835 of the Ad2 genome with a deletion of the complete sequence of E4orf6/7 intron 1 (1275 bp, SEQ ID NO:1).

In some embodiments, the modified adenovirus E4 coding region comprises a partial deletion of E4orf6/7 intron 1, wherein up to 10%, 20, 30%, 40%, 50%, 60%, 70%, 80%, 90% or 95% of SEQ ID NO:1 is deleted, and/or a partial deletion of E4orf6/7 intron 2, wherein up to 10%, 20, 30%, 40%, 50%, 60%, 70%, 80%, 90% or 95% of SEQ ID NO:2 is deleted.

In some embodiments, the modified adenovirus E4 coding region comprises a codon optimized nucleotide sequence (SEQ ID NO:7) encoding adenovirus E4orf6/7 protein (SEQ ID NO:8).

In some embodiments, the modified adenovirus E4 coding region comprises a codon optimized nucleotide sequence (SEQ ID NO:7) encoding adenovirus E4orf6/7 protein (SEQ ID NO:8) and a codon optimized nucleotide sequence (SEQ ID NO:25) encoding adenovirus E4orf6 protein (SEQ ID NO:26).

In some embodiments, the modified adenovirus E4 coding region encodes Ad2 E4orf6/7. In some embodiments, the modified adenovirus E4 coding region comprises SEQ ID NO:17. In some embodiments, the modified adenovirus E4 coding region comprises SEQ ID NO:18.

In some embodiments, the modified adenovirus E2a coding region contains a codon-modified, or un-modified sequence encoding adenovirus E2a protein with a partial or complete deletion of early E2a intron 1.

In some embodiments, the modified adenovirus E2a coding region contains a codon-modified, or un-modified, sequence encoding adenovirus E2a protein with a partial or complete deletion of late E2a intron 1.

In some embodiments, the modified adenovirus E2a coding region contains a codon-modified, or un-modified, sequence encoding adenovirus E2a protein with a partial or complete deletion of E2a intron 2.

In some embodiments, the modified adenovirus E2a coding region contains a codon-modified, or un-modified sequence encoding adenovirus E2a protein with a partial or complete deletion of early E2a intron 1 and a partial or complete deletion of E2a intron 2.

In some embodiments, the modified adenovirus E2a coding region contains a codon-modified, or un-modified sequence encoding adenovirus E2a protein with a partial or complete deletion of late E2a intron 1 and a partial or complete deletion of E2a intron 2.

In some embodiments, the modified adenovirus E2a coding region comprises a partial deletion of later-E2a intron 1 and/or later-E2a intron 2, wherein up to 10%, 20, 30%, 40%, 50%, 60%, 70%, 80%, 90% or 95% of the later-E2a intron 1 and/or later-E2a intron 2 is deleted.

In some embodiments, the modified adenovirus E2a coding region has a total size of 1600-3500, 1600-3000, 1600-2500, 1600-2000, 1600-1800 or 1600-1700 bp and encodes a functional E2a protein that is capable of providing the required E2a functions for parvovirus production in a non-E2a expressing host cell.

In some embodiments, the modified adenovirus E2a coding region comprises a sequence corresponding to nucleotide sequence 22233-27575 of the Ad2 genome with (a) a partial or complete deletion of early-E2a or later-E2a intron 1, (b) a partial or complete deletion of E2a intron 2, or both (a) and (b). In some embodiments, the modified adenovirus E2a coding region comprises a sequence corresponding to nucleotide sequence 22233-27575 of the Ad2 genome with a 1897 bp deletion (SEQ ID NO:3) that encompasses the complete sequence of later-E2a intron 1 and later-E2a intron 2. In some embodiments, the modified adenovirus E2a coding region comprises a partial deletion of later-E2a intron 1 and/or later-E2a intron 2, wherein up to 10%, 20, 30%, 40%, 50%, 60%, 70%, 80%, 90% or 95% of the SEQ ID NO:3 is deleted.

In some embodiments, the modified adenovirus E2a coding region comprises (a) a partial or complete deletion of later a sequence encoding a codon optimized adenovirus E2a protein E2a intron 1, (b) a partial or complete deletion of later E2a intron 2, and (c) a codon modified nucleotide sequence encoding an adenovirus E2a protein.

In some embodiments, the modified adenovirus E2a coding region comprises SEQ ID NO:4.

In some embodiments, the adenovirus VA RNA coding region comprises sequences encoding adenovirus VA1, adenovirus VAII or both. In some embodiments, the adenovirus VA RNA coding region comprises sequences encoding Ad2 VA1, Ad2 VAII or both. In some embodiments, the adenovirus VA RNA coding region comprises SEQ ID NO:16.

In some embodiments, the AAV Rep coding region comprises SEQ ID NO:20.

In some embodiments, the AAV Cap coding region comprises SEQ ID NO:21.

In some embodiments, the one or more regulatory elements comprise an adenovirus E4 promoter operably linked to the modified adenovirus E4 coding region, an adenovirus E2a promoter operably linked to the modified adenovirus E2a coding region, and a modified AAV P5 promoter operably linked to the sequences that encode AAV Rep and Cap proteins. In some embodiments, the adenovirus E4 promoter comprises SEQ ID NO:22. In some embodiments, the adenovirus E2a promoter comprises SEQ ID NO:23. In some embodiments, the modified AAV P5 promoter comprises SEQ ID NO:16.

In some embodiments, the expression construct further comprises a nucleotide sequence encoding an adenovirus Ela protein and/or a nucleotide sequence encoding an adenovirus E1b protein.

In some embodiments, the expression construct designed for single-plasmid parvovirus production system has a size of less than 20 kb, 19 kb, 18 kb, 16 kb, 15 kb, 14 kb, 13 kb, 12 kb, 11 kb, 10 kb, 9 kb, 8 kb, or 7 kb.

In some embodiments the expression construct designed for single-plasmid parvovirus production system has a size in the range of 7-25 kb, 7-20 kb, 7-18 kb, 7-16 kb, 7-14 kb, 7-12 kb, 7-10 kb, 10-25 kb, 10-20 kb, 10-18 kb, 10-16 kb, 10-14 kb, 10-12 kb, 12-25 kb, 12-20 kb, 12-18 kb, 12-16 kb, 12-14 kb, 14-25 kb, 14-20 kb, 14-18 kb, 14-16 kb, 16-25 kb, 16-20 kb, 16-18 kb, 18-25 kb, 18-20 kb, or 20-25 kb.

In some embodiments, the expression construct is a plasmid having a total size of 10-15 kb, 10-20 kb, 10-25 kb, 15-20 kb, 15-25 kb or 20-25 kb.

Preferred Embodiments of Expression Constructs for One-Plasmid AAV Production System

In some embodiments, the expression construct is a plasmid for one-plasmid AAV production and comprises a modified adenovirus E4 coding region comprising a coding sequence for E4orf6 and a coding sequence for E4orf6/7, a modified adenovirus E2a coding region comprising a coding sequence for E2a, a sequence that encodes adenovirus VAI RNA, a sequence that encodes an AAV Rep protein, a sequence that encodes an AAV Cap protein, and a sequence that encodes a recombinant AAV genome, wherein the expression construct has a size in the range of 7-19 kb.

In some embodiments, the expression construct is a plasmid for one-plasmid AAV production and comprises a modified adenovirus E4 coding region comprising SEQ ID NO:17 or SEQ ID NO:18, a modified adenovirus E2a coding region comprising SEQ ID NO:4, a sequence that encodes adenovirus VAI RNA coding region comprising SEQ ID NO:16, a regulatory region comprising SEQ ID NO:19, an AAV Rep coding region comprising SEQ ID NO:20, an AAV Cap coding region comprising SEQ ID NO:21, and a sequence encoding a recombinant AAV genome, wherein the expression construct has a size in the range of 7-19 kb.

In another embodiment, the present application provides one or more cells, each containing one or more of the expression constructs described herein. In another embodiment, the present application provides one or more cell lines, each stably transformed with one or more of the expression constructs described herein.

In another aspect, the present application provides a kit for producing recombinant AAV particles. In one embodiment, the kit comprises a plasmid comprising the expression construct of the present application.

In a particular embodiment, the kit further comprises instructions for use, e.g., instructions for use in a method described herein. In some embodiments, the kit further comprises one or more tubes or other types of containers for cell lysate (e.g., Eppendorf tubes) and/or one or more tubes or other types of containers for waste generated.

### III. Methods for Producing Recombinant Parvovirus

Another aspect of the present application relates to methods for producing a recombinant parvovirus particle using the expression constructs of the present application. The method comprises the step of introducing the expression construct of the present application into a host cell that provides the necessary helper function for the parvovirus production, incubating the host cell harboring the expression cassette for a desired period of time to product recombinant parvovirus particles, and harvesting recombinant parvovirus particles after the incubation period. In some embodiments, the parvovirus is AAV.

In some embodiments, the method comprises the step of (1) introducing into a host cell (A) the expression construct of the triple-plasmid parvovirus production system, (B) a parvovirus trans construct that is capable of expressing parvovirus proteins necessary for the production of recombinant parvovirus, and (C) a parvovirus cis construct containing a sequence encoding the recombinant parvovirus genome, (2) incubating the host cell harboring the three constructs for a desired period of time to produce recombinant parvovirus particles, and (3) harvesting recombinant parvovirus particles after the incubation period. In some embodiments, constructs A, B and C are cotransfected into the host cell at a A:B:C molar ratio of 1-10:1-10:1-10. In some embodiments, constructs A, B and C are cotransfected into the host cell at a A:B:C molar ratio of 1:1:1. This method is often referred to as triple-transfection method. In some embodiments, the parvovirus is AAV and the trans construct comprises an AAV Rep coding region and an AAV Cap coding region.

In some embodiments, the method comprises the step of (1) introducing into a host cell (A) an expression construct (construct A) of the dual-plasmid parvovirus production system that contains a modified adenovirus E4 coding region that encodes E4orf6/7, a modified adenovirus E2a coding region that encodes an E2a protein, an adenovirus VA RNA coding region, a parvovirus protein coding region, and one or more regulatory elements that allow expression of the adenovirus E4orf6/7, E2a, VA RNA and the parvovirus proteins, and (B) an expression construct (construct B) comprising a sequence encoding a recombinant parvovirus genome (2) incubating the host cell harboring the constructs for a desired period of time to produce recombinant parvovirus particles, and (3) harvesting the recombinant parvovirus particles after the incubation period. In some embodiment, the recombinant parvovirus is a rAAV. In some embodiments, constructs A and B are cotransfected into the host cell at a A:B molar ratio in the range of 1:10 to 10:1. In some embodiments, constructs A and B are cotransfected into the host cell at a A:B molar ratio of 1:1, 1:2, 1:3, 2:1 or 3:1.

In some embodiments, the method comprises the step of (1) introducing into a host cell (A) an expression construct (construct A) of the dual-plasmid parvovirus production system that contains a modified adenovirus E4 coding region that encodes E4orf6/7, a modified adenovirus E2a coding region that encodes an E2a protein, an adenovirus VA RNA coding region, a sequence encoding a recombinant parvovirus genome, and one or more regulatory elements that allow expression of the adenovirus E4orf6/7, E2a, VA RNA, and (B) an expression construct (construct B) comprising parvovirus protein coding region (2) incubating the host cell harboring the constructs for a desired period of time to produce recombinant parvovirus particles, and (3) harvesting the recombinant parvovirus particles after the incubation period. In some embodiments, the recombinant parvovirus is a rAAV. In some embodiments, constructs A and B are cotransfected into the host cell at a A:B molar ratio in the range of 1:10 to 10:1. In some embodiments, constructs A and B are cotransfected into the host cell at a A:B molar ratio of 1:1, 1:2, 1:3, 2:1 or 3:1.

In some embodiments, the method comprises the step of (1) introducing into a host cell the expression construct of the single-plasmid parvovirus production system, (2) incubating the host cell harboring the expression cassette for a desired period of time to product recombinant parvovirus particles, and (3) harvesting recombinant parvovirus particles after the incubation period. In some embodiments, the expression construct is a plasmid for production of recombinant AAV. In some embodiments, the plasmid does not encode for adenovirus Ela and E1b proteins and the host cell expresses adenovirus Ela and E1b proteins endogenously.

A host cell for producing parvovirus particles can contain the expression construct of the present application in the form of e.g., episomal plasmids. The expression construct of the present application may also be stably integrated into the host cell genome. Further, the host cell can constitute an expression system for producing adenovirus proteins, such as Ela and E1b, that are required for parvovirus production. Examples of host cells include, but are not limited to, microorganisms, yeast cells, insect cells and animal cells. In some embodiments, the host cell is a mammalian host cell, such as human HuH7 and HEK293 cells, Chinese hamster ovary cells (“CHO”), and baby hamster kidney (“BHK”) cells. Mammalian cells suitable for carrying out the present application include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, HuH7, HEK293 and NS-1 cells. The term “host cell” includes the progeny of the original cell which has been transfected. It is understood that the progeny of a single parental cell may not necessarily be completely identical in morphology or in genomic or total DNA complement as the original parent, due to natural, accidental, or deliberate mutation. A “mammalian host cell” is a cell originally derived from a mammal or is a progeny cell thereof.

In one embodiment, the host cell expresses Ad Ela and E1b protein. In certain preferred embodiments, the host cell is an HEK-293 cell that expresses adenovirus Ela and E1b.

In some embodiments, the expression construct of the present application and other expression constructs (if any) are transfected into HEK293 cells (available from ATCC®) via CaPO₄-mediated transfection, lipids or polymeric molecules such as Polyethylenimine (PEI). The HEK293 cells are then incubated for at least 60 hours to allow for recombinant parvovirus particle production.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### IV. Kits

Another aspect of the present application relates to a kit for producing a recombinant parvovirus. In some embodiments, the kit comprises an expression construct of the present application and an instruction on how to use the expression construct.

In some embodiments, the expression construct comprises (1) a modified adenovirus E4 coding region that encodes E4 open reading frame 6/7, (2) a modified adenovirus E2a coding region that encodes an E2a protein, (3) an adenovirus VA RNA coding region, (4) a parvovirus protein coding region, (5) a recombinant parvovirus cloning region, and (6) one or more regulatory elements that allow expression of the E4 open reading frame 6/7; the E2a protein, the adenovirus VA RNA, and parvovirus proteins in a host cell. In some embodiments, the recombinant parvovirus cloning region comprises a cloning site for insertion of a recombinant parvovirus sequence comprising (1) an expression cassette comprising a gene of interest (GOI) operably linked to a regulatory element and (2) at least one ITR at one side of the expression cassette. In some embodiments, the recombinant parvovirus sequence comprises two ITRs, one at each end of the expression cassette. In some embodiments, a recombinant parvovirus cloning region comprises (1) a cloning site for insertion of a sequence comprising an expression cassette comprising a GOI operably linked to a regulatory element, and (2) at least one ITR at one side of the cloning site. In some embodiments, the recombinant parvovirus sequence comprises two ITRs, one at each side of the cloning site. bIn some embodiments, a recombinant parvovirus cloning region comprises (1) an expression cassette comprising a cloning site for insertion of a GOI and a regulatory element in the proximity of the cloning site and is capable of being operably linked to the GOI when the GOI is inserted at the cloning site, and (2) at least one ITR at one side of the expression cassette. In some embodiments, the recombinant parvovirus sequence comprises two ITRs, one at each side of the expression cassette.

In some embodiments, the expression construct comprises (1) a modified adenovirus E4 coding region that encodes E4 open reading frame 6/7, (2) a modified adenovirus E2a coding region that encodes an E2a protein, (3) an adenovirus VA RNA coding region, (4) an AAV Rep coding region, (5) an AAV Cap coding region, (6) a sequence encoding a recombinant AAV genome, comprising an expression cassette having a nucleotide sequence encoding a transgene and a regulatory element operably linked to the nucleotide sequence, and at least one ITR at one end of the expression cassette, and (7) one or more regulatory elements that allow expression of the E4 open reading frame 6/7; the E2a protein, the adenovirus VA RNA, the AAV Rep protein, and the AAV Cap protein in a host cell. In some embodiments, the expression cassette in the recombinant AAV genome is flanked by an AAV ITR on each end.

In some embodiments, the kit further comprises a reagent for transfection of the expression construct.

### EXAMPLES

### Example 1: Construction of AAV Helper Constructs

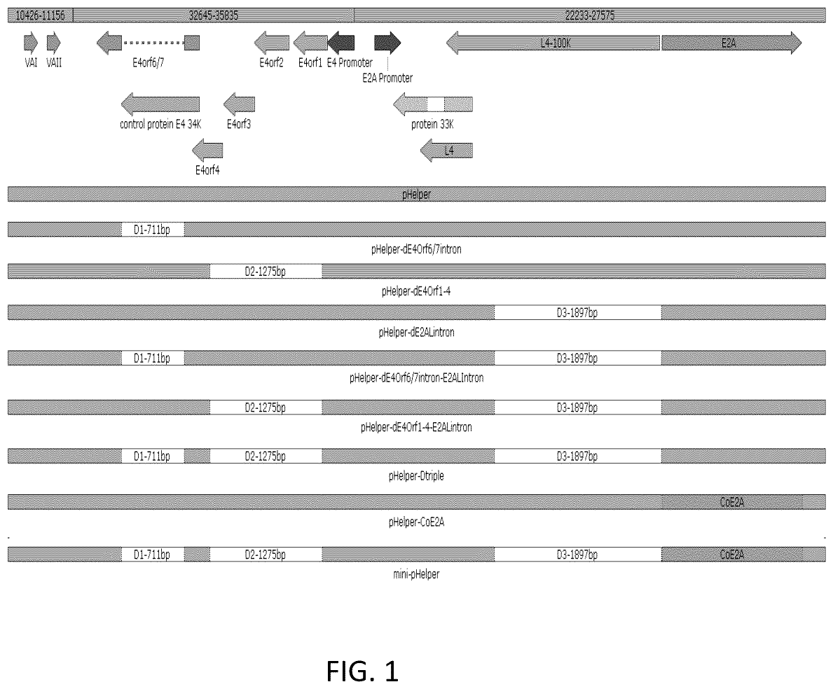

To examine the dispensability of Ad sequences for packaging AAV vectors, a series of Ad-based deletion mutants were constructed in order to reduce the size of plasmids and increase helper functions for producing recombinant AAV virus particles. FIG. 1 depicts a series of expression cassettes with regard to their arrangement of adenovirus (Ad2) coding regions and surrounding deletions relative to Ad2 nucleotide sequence coordinates.

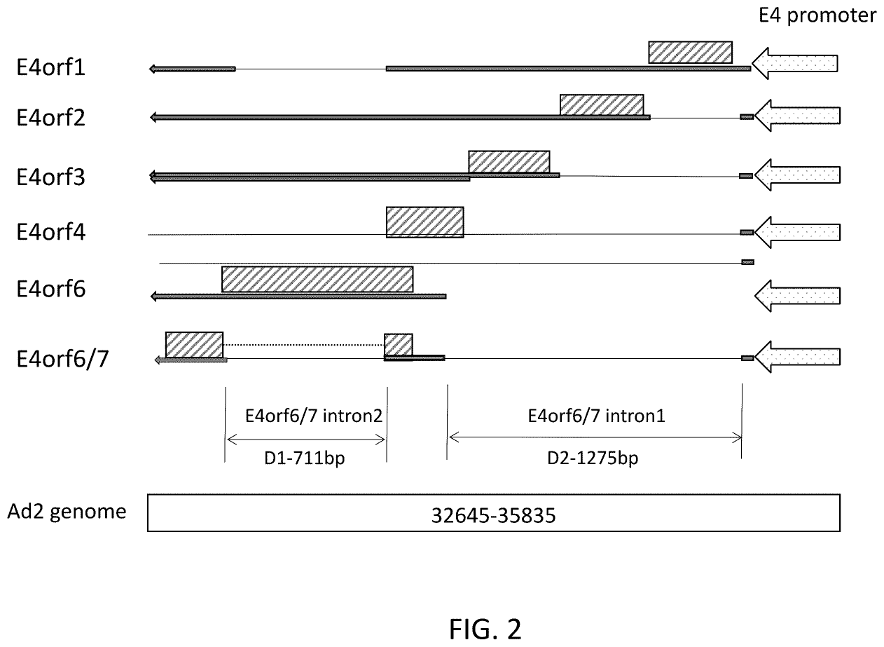

FIG. 2 shows an expanded map depicting the arrangement of Ad E4orfs and the surrounding deletions depicted in FIG. 1 relative to the Ad2 genomic sequences.

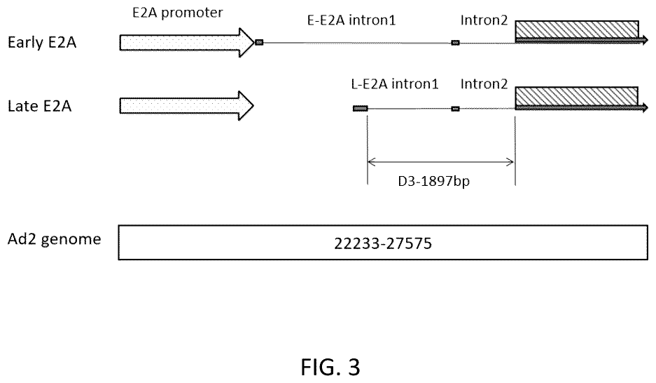

FIG. 3 shows an expanded map depicting the Ad2 E2a promoter and Ad2 mRNAs expressed at early and late times in infection, including the surrounding deletions depicted in FIG. 1 relative to the Ad genomic sequences.

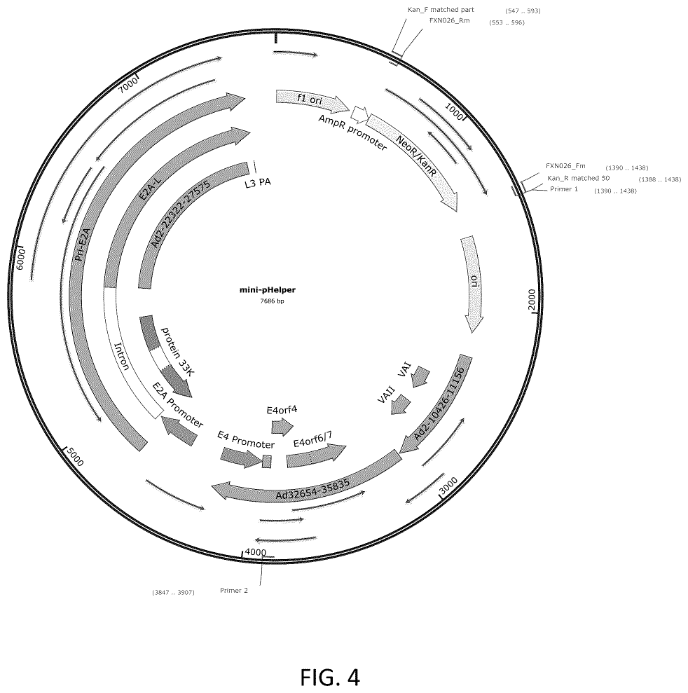

FIG. 4 shows an exemplary helper plasmid, mini-pHelper plasmid (SEQ ID NO:6) that contains an embodiment of the expression cassette of the present application. The plasmid contains the mini-pHelper Ad helper gene arrangement shown in FIG. 1. The mini-pHelper backbone plasmid includes a codon-optimized Ad E2a coding region sequence (SEQ ID NO:4) encoding the wild-type Ad E2a amino acid sequence set forth in SEQ ID NO:5.

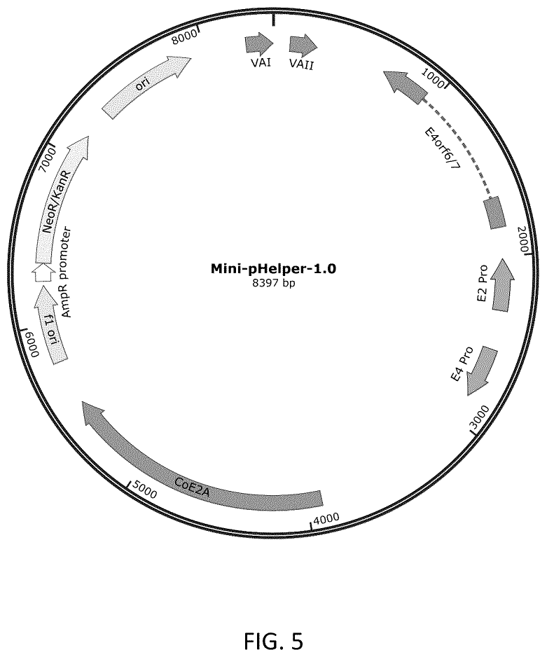

FIG. 5 shows another exemplary helper plasmid, mini-pHelper-1.0 plasmid (SEQ ID NO:10) that contains another embodiment of the expression cassette of the present application. The plasmid contains the mini-pHelper Ad helper gene arrangement shown in FIG. 1.

### Example 2: Identification of Adenovirus Regions Dispensable for AAV Production

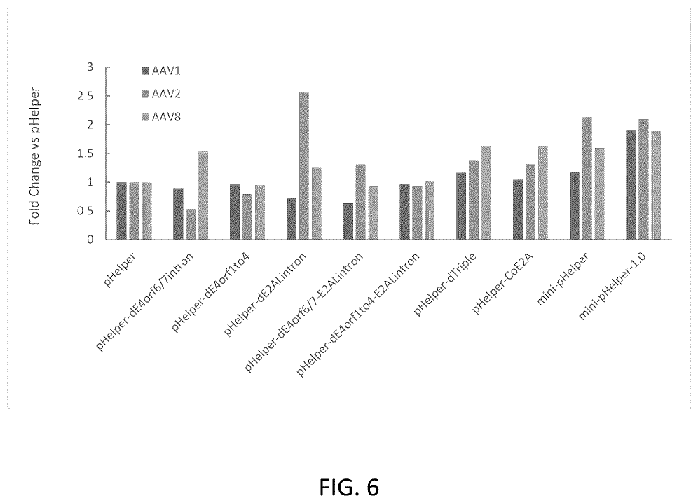

FIG. 6 shows the effects of the deletions or combinations of deletions in FIG. 1 on the ability of the expression cassette to sufficiently provide helper functions for adeno-associated virus (AAV) packaging. The production titers of rAAV were obtained using a triple plasmid transfection system: an ITR containing plasmid (such as pAAV-CAG-EGFP), an AAV helper plasmid (such as pRep2-AAV1, pRep2-AAV2 or pRep2-AAV9), and an Ad helper plasmid Ad with an Ad helper gene arrangement shown in FIG. 1 (such as pHelper, pHelper-dE4Orf6/7intron,mini-pHelper1.0 etc.) that provided the required adenoviral functions. All the AAV vectors were produced in HEK 293T cells. The Y-axis showed the fold increase of using different novel pHelpers compared with the original pHelper.

### Example 3: Construction of Mini-Helper Plasmids for AAV Library Production

FIG. 7 shows schematics comparing the original triple-plasmid transfection system with the mini-pHelper based-production system. Green—Ad genes, blue—AAV genes, pink transgene elements. In the original triple-plasmid system, the total mass of three plasmids (pHelper, pRep&Cap, GOI) is between 24.1-28.1 kb. In the mini-phelper plasmid system, the total mass of three plasmids was reduced to 20.9-24.9 kb. There are two different versions of dual-plasmid system (V1, the AAV genes (Rep and Cap) were inserted into mini-pHelper; V2, the transgene elements were inserted into mini-pHelper. The total mass of V1 and/or V2 is 17.9-21.9 kb. In the one plasmid system, both the AAV genes and the transgene elements were inserted into mini-pHelper. The total mass of the plasmid is 13.9-17.9 kb.

Construction of mini-pHelper-AAV2 (SEQ ID NO:10): Mini-pHelper1.0 plasmid was digested with ClaI. AAV helper genes (Rep and Cap) were PCR amplified and assembled into ClaI site.

Construction of mini-pHelper-CMV-EGFP (SEQ ID NO:11): Mini-pHelper1.0 plasmid (SEQ ID NO:9) was digested with PmeI and used as backbone. The insert ITR-CMV-EGFP-ITR cassette was digested from pAAV-CMV-EGFP with SbfI. The backbone and insert were ligated with T4 DNA ligase.

Construction of pAAVone-AAV2-CMV-EGFP (SEQ ID NO:12): Mini-pHelper-AAV2 plasmid was digested with PmeI and used as backbone. The insert ITR—CMV-EGFP-ITR cassette was digested from pAAV-CMV-EGFP with SbfI. The backbone and insert were ligated with T4 DNA ligase.

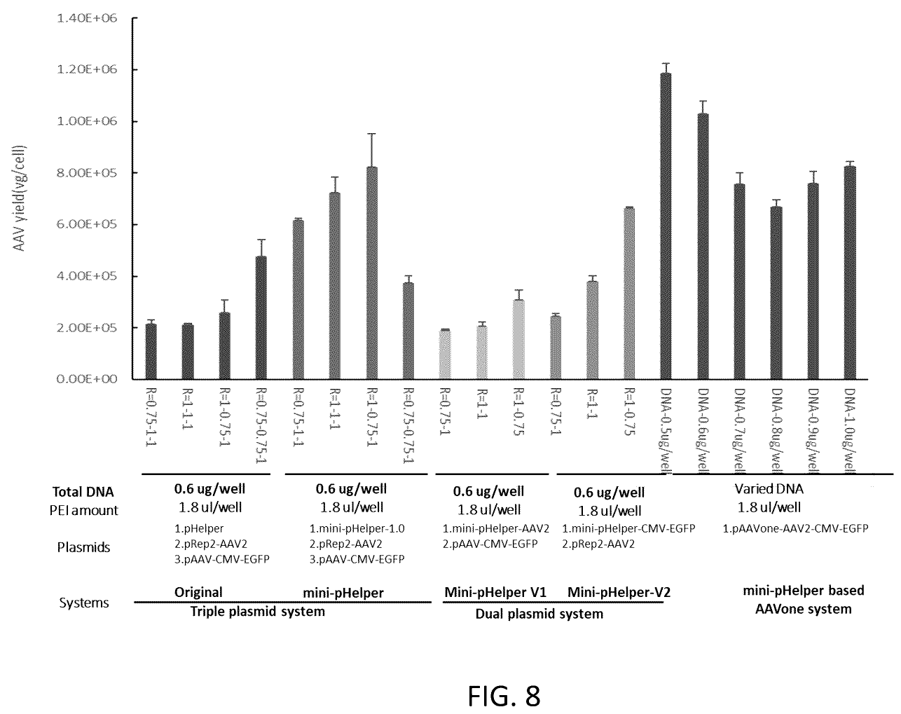

FIG. 8 shows the package efficiency of mini-helper based AAV production system in adherent HEK 293 T cells. The production titers of rAAV were obtained in HEK 293T cells at 72 h post transfection. In the triple plasmid transfection systems, 4 different molecular ratios of three plasmids (pHelper or mini-phelper-1.0):pRep2-AAV2:pAAV-CMV-EGFP) with total DNA mass of 0.6 ug/well were transfected into HEK 293T cells used fixed amount of polyethylenimine (PEI) (1.8 ul/well). For the dual plasmid system, 3 different molecular ratios were evaluated. In the one plasmid system, different amounts of plasmids were transfected with fixed amount of PEI.

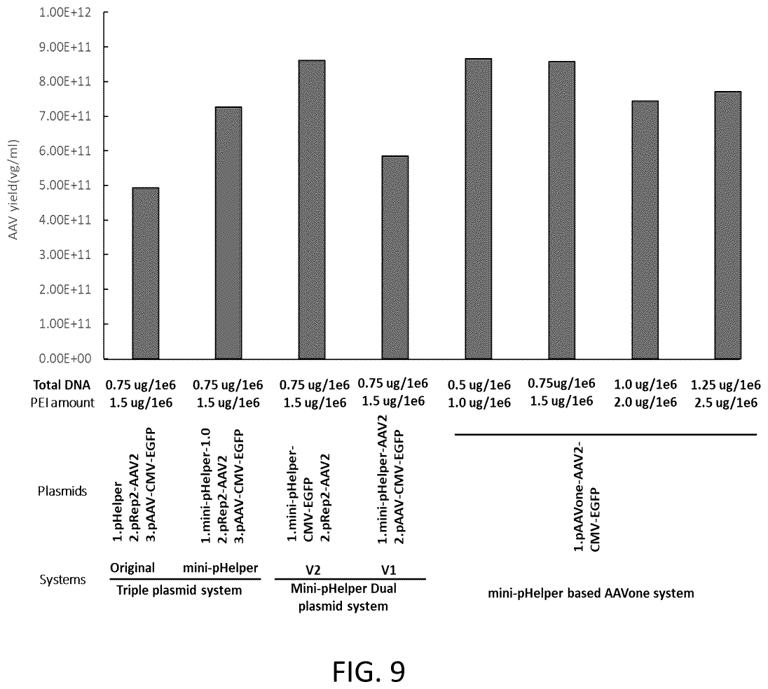

FIG. 9 shows the package efficiency of mini-helper based AAV production system in suspension cultured HEK 293T cells. The production titers of rAAV were obtained at 72 h post transfection. In the triple plasmid transfection system, the ratio of three plasmid is 1:1:1 and the total DNA is 0.75 ug/10⁶ cells, In the dual plasmid transfection systems, the ratio of two plasmids is 1:1 and the total DNA is 0.75 ug/10⁶ cells. In the one plasmid system, different amounts of plasmids were transfected with fixed PEI:DNA ratio of 2.

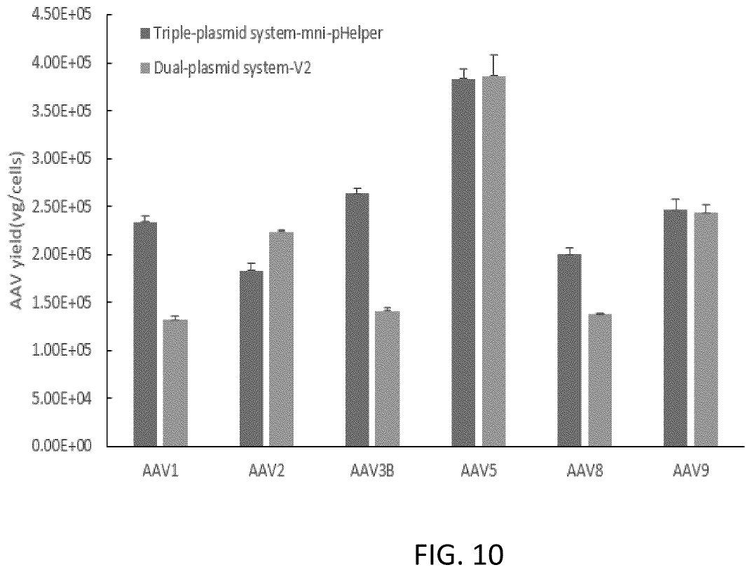

FIG. 10 shows the package efficiency of mini-helper based dual-plasmid V2 system for different AAV serotypes. In the dual-plasmid V2 system, the transgene elements were inserted into mini-pHelper. AAV production system in suspension cultured HEK 293T cells. The production titers of rAAV were obtained at 72 h post transfection. In the triple plasmid transfection system, the molecular ratio of three plasmids is 1:1:1 and the total DNA is total DNA mass of 0.6 ug/well. In the dual plasmid transfection systems, the molecular ratio of two plasmids is 1:1 and the total DNA is total DNA mass of 0.6 ug/well.

### Example 4: Use of Mini Helper for AAV Library Production

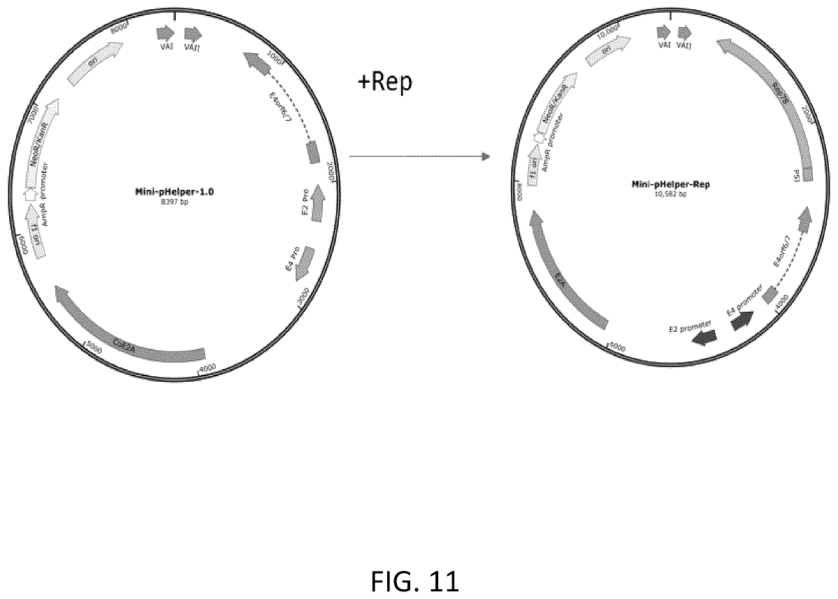

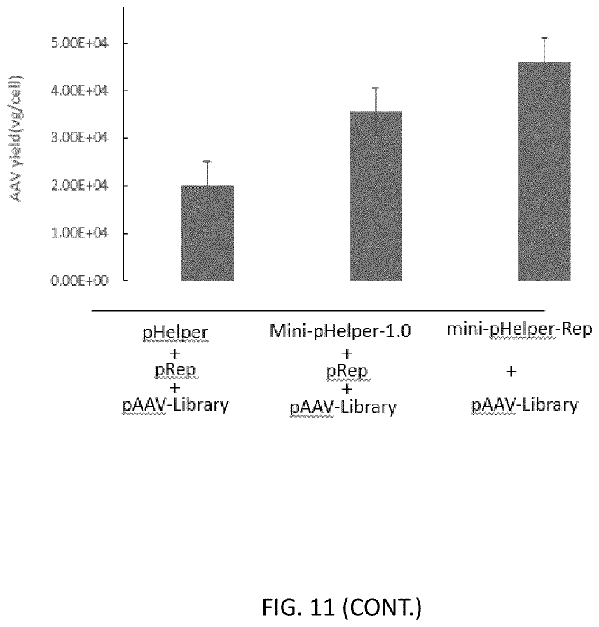

FIG. 11 shows the use of mini-helper for AAV library production. The production titers of rAAV were obtained in HEK 293T cells at 72 h post transfection. The pAAV-Library plasmid was used as 200 copies/cell. pHelper and pRep, mini-pHelper-1.0 and mini-pHelper-Rep (SEQ ID NO:13) were used as 10⁵ copies/cell. The PEI was used as 1.5 ug/well. Mini-pHelper-Rep: Mini-pHelper-AAV2 plasmid was digested with SwaI and SnaBI to remove the Cap gene.

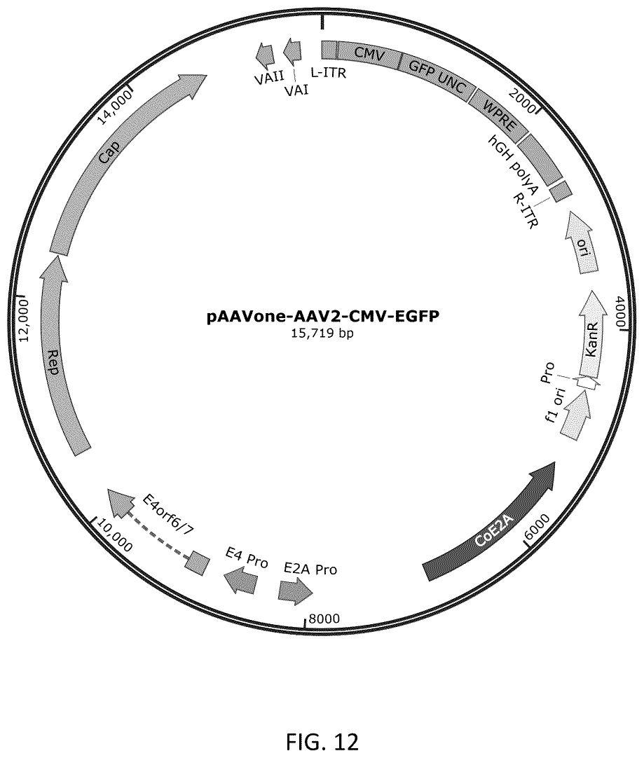

FIG. 12 shows an exemplary helper plasmid pAAVone-AAV2-CMV-EGFP (SEQ ID NO:12) that contains another embodiment of the expression cassette of the present application. The plasmid contains the mini-pHelper Ad helper gene arrangement shown in FIG. 1, AAV Rep and Cap genes, and the sequence of a recombinant AAV genome (ITR-CMV-EGFP-PolyA-ITR)

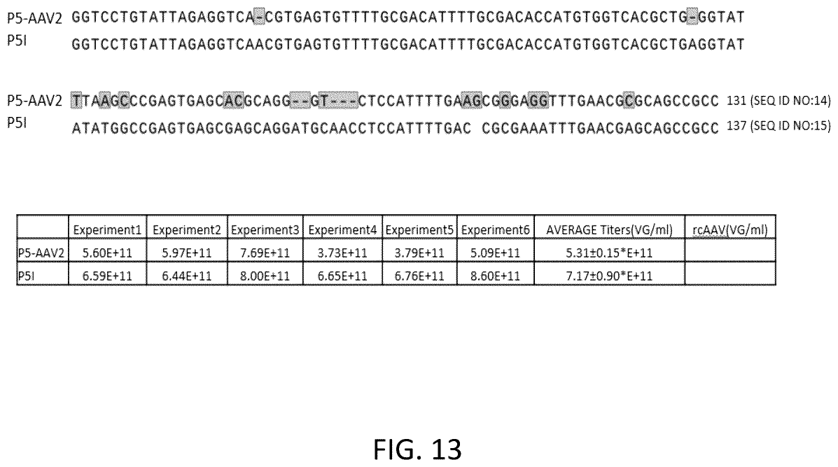

FIG. 13 shows the sequences of a wild-type AAV2 P5 promoter P5 (P5-AAV2, SEQ ID NO:14) and a modified AAV P5 promoter P5 (PSI, SEQ ID NO:15) and their effect on package efficiency of AAVone system. The total DNA is total DNA mass of 0.6 ug/well. The production titers of rAAV were obtained at 72 h post transfection.

### Example 5: Package Efficiency of Mini-Helper-1.0

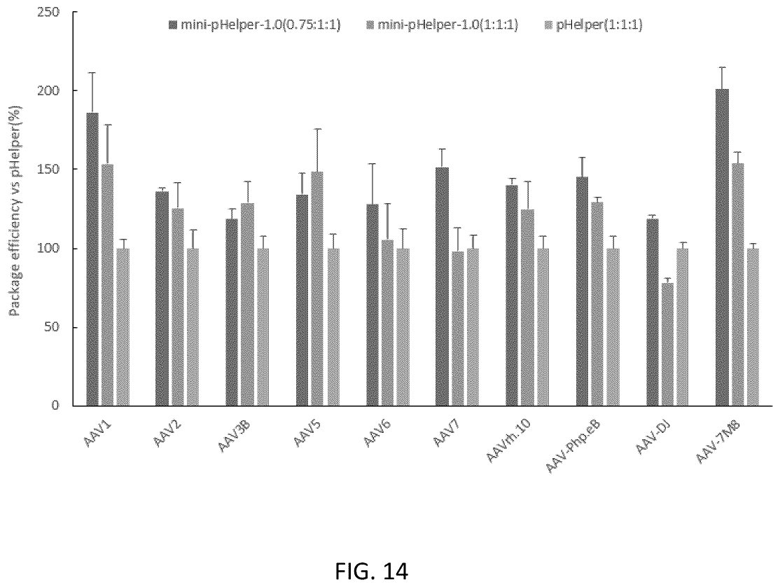

FIG. 14 shows the package efficiency of mini-helper-1.0 plasmid compared with original pHelper plasmid for different AAV serotypes. The production titers of rAAV were obtained at 72 h post transfection. In the triple plasmid transfection system, the molecular ratio of three plasmids is 1:1:1 or 0.75:1:1 and the total DNA is total DNA mass of 0.6 ug/well were transfected into HEK 293T cells used fixed amount of polyethylenimine (PEI) (1.8 ul/well).

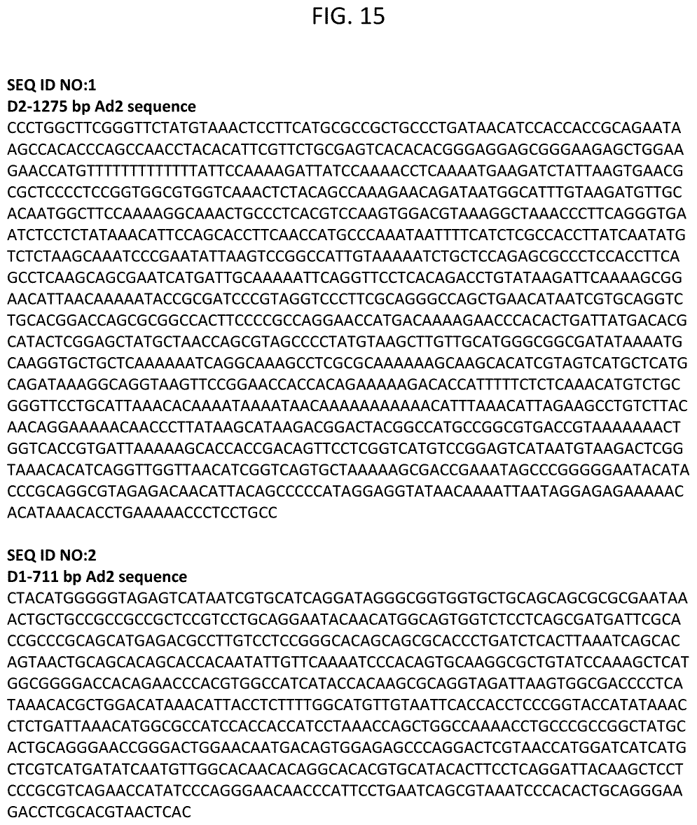

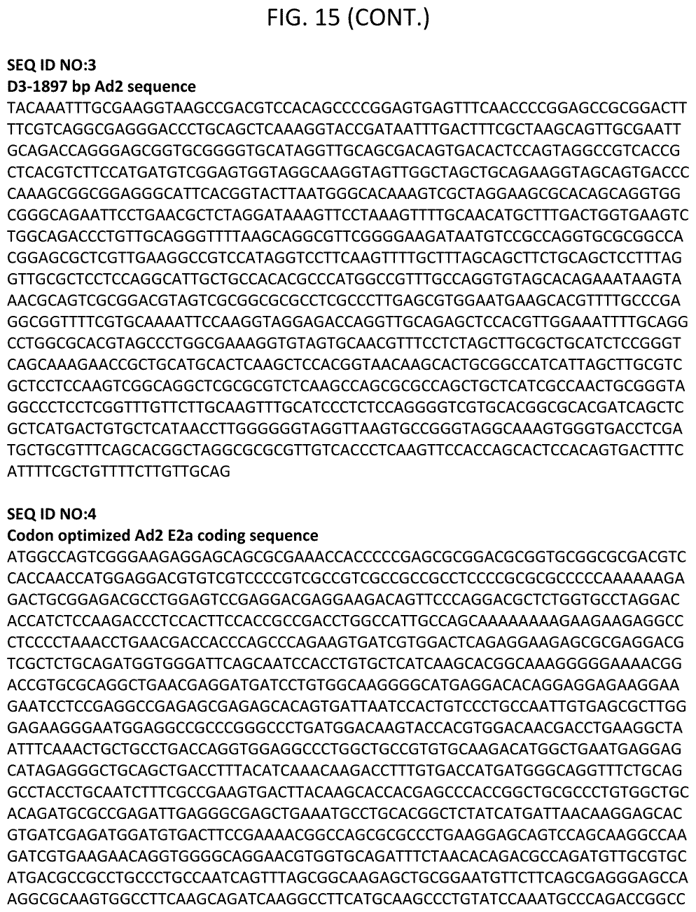

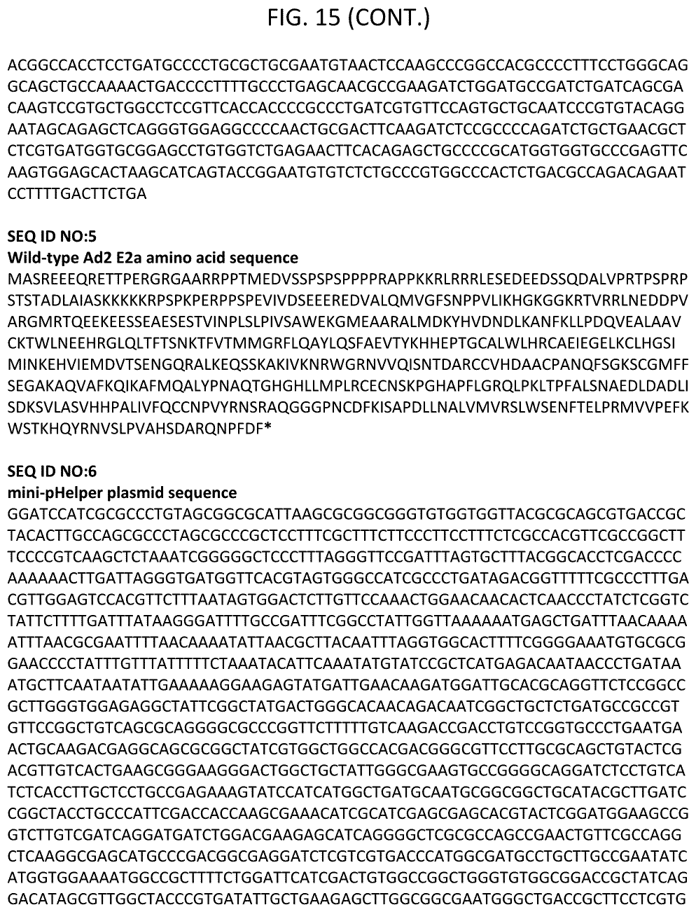

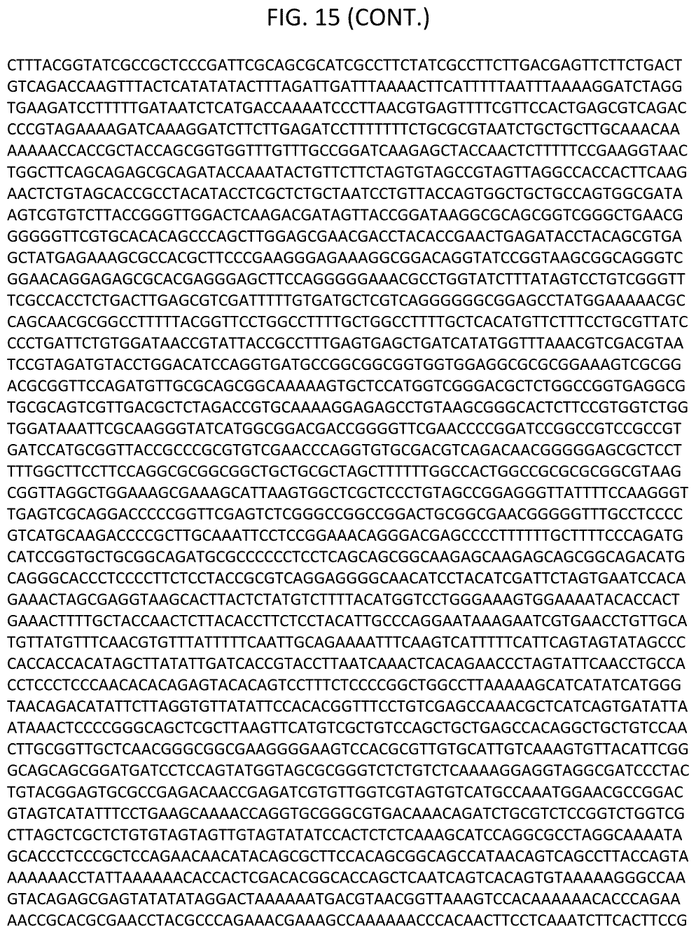

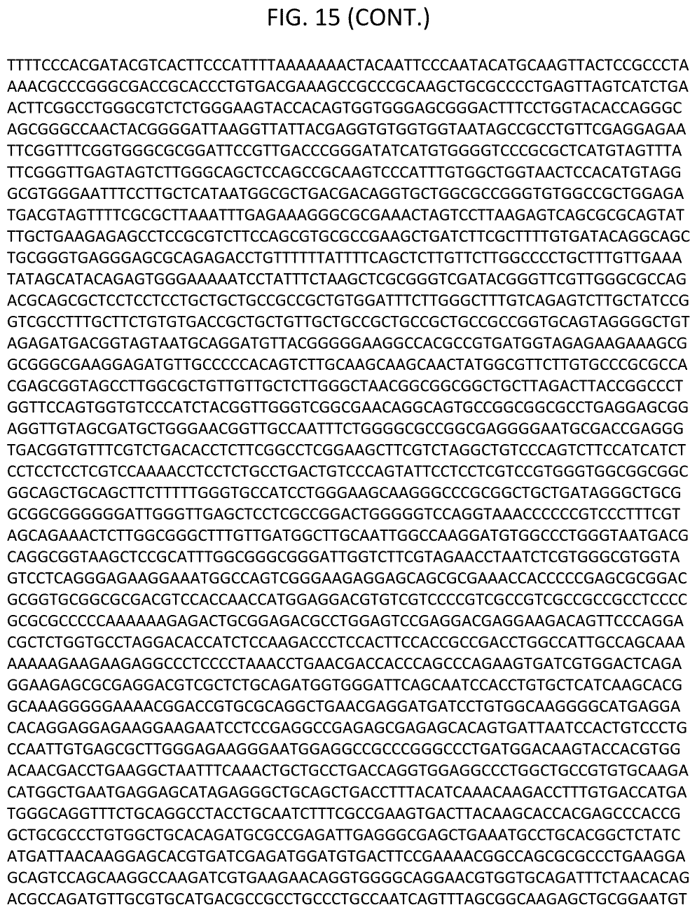

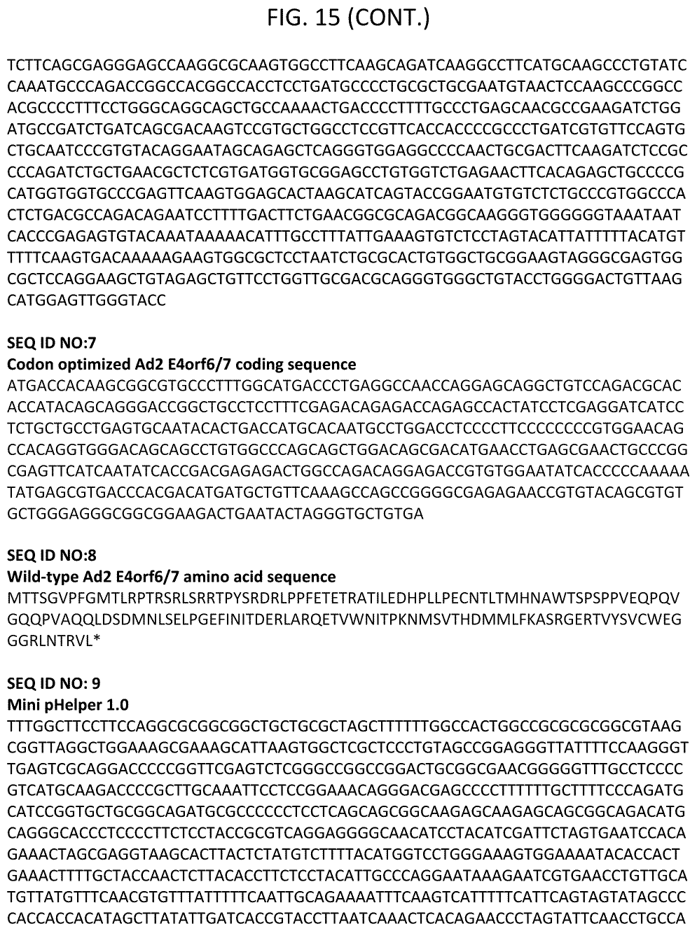

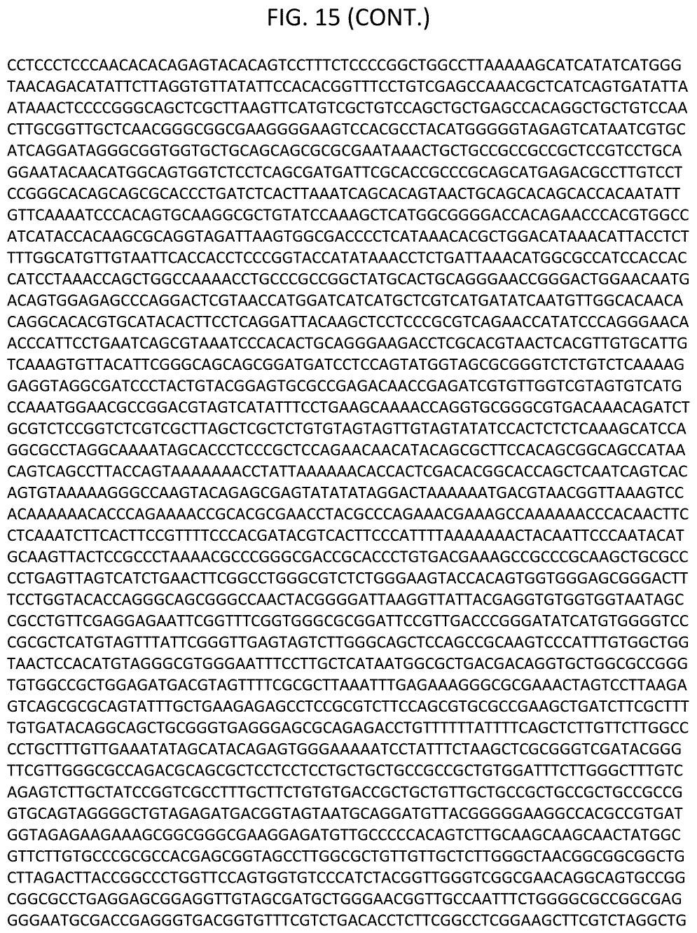

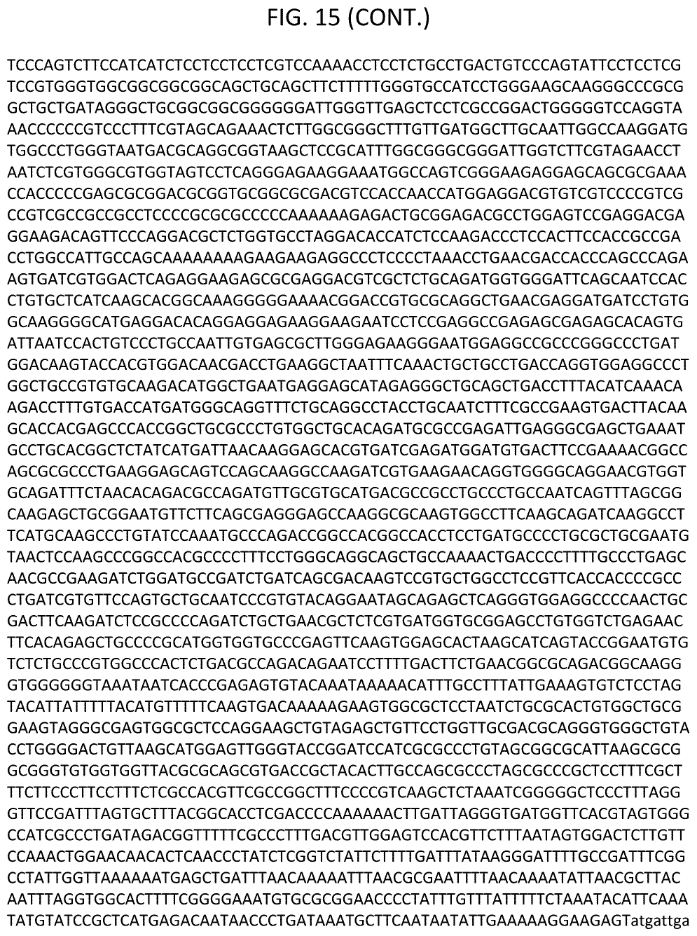

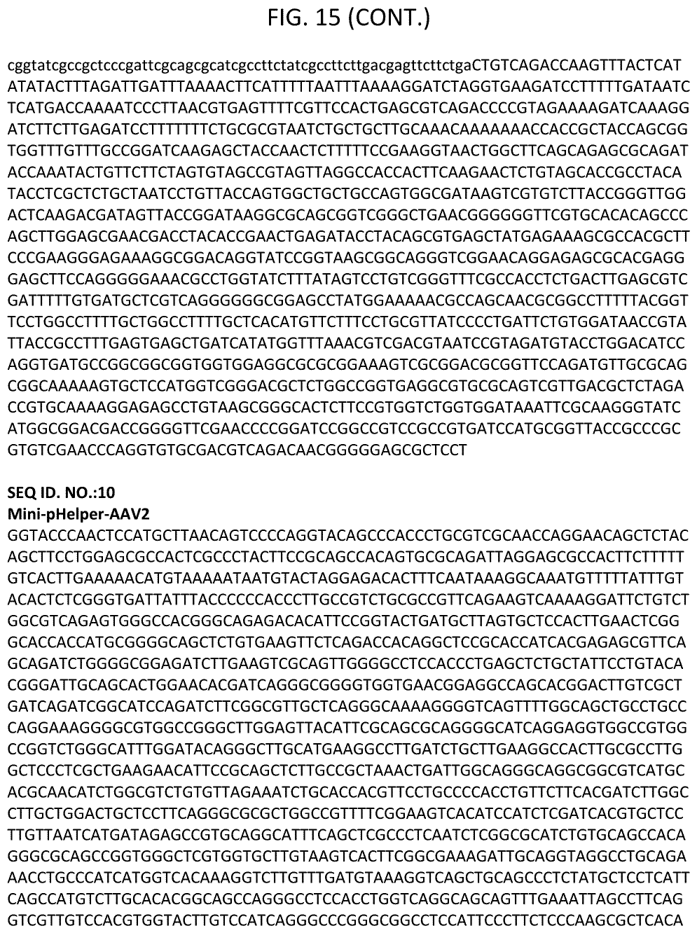

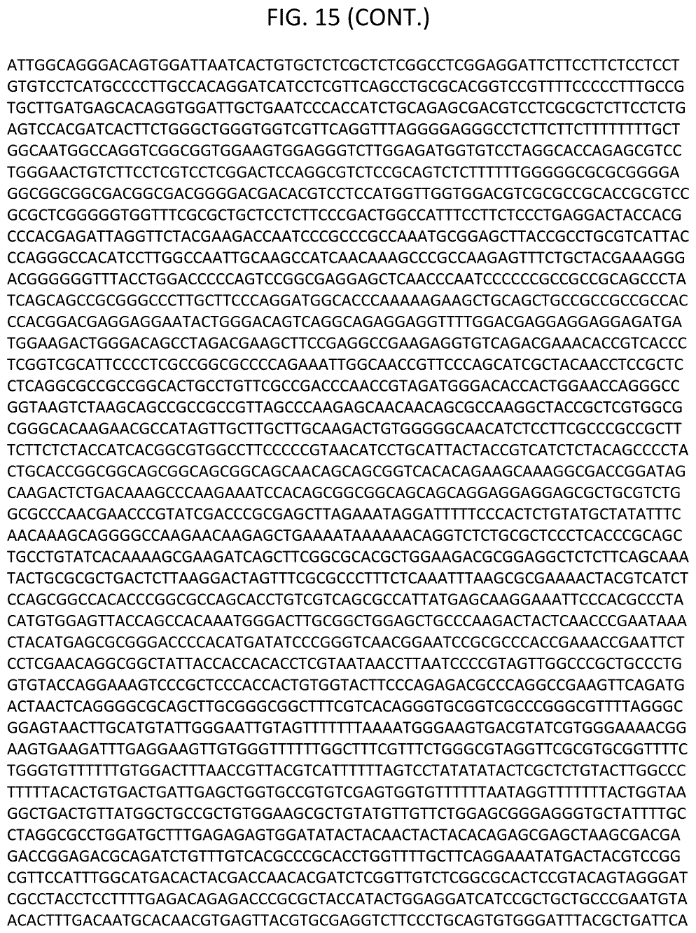

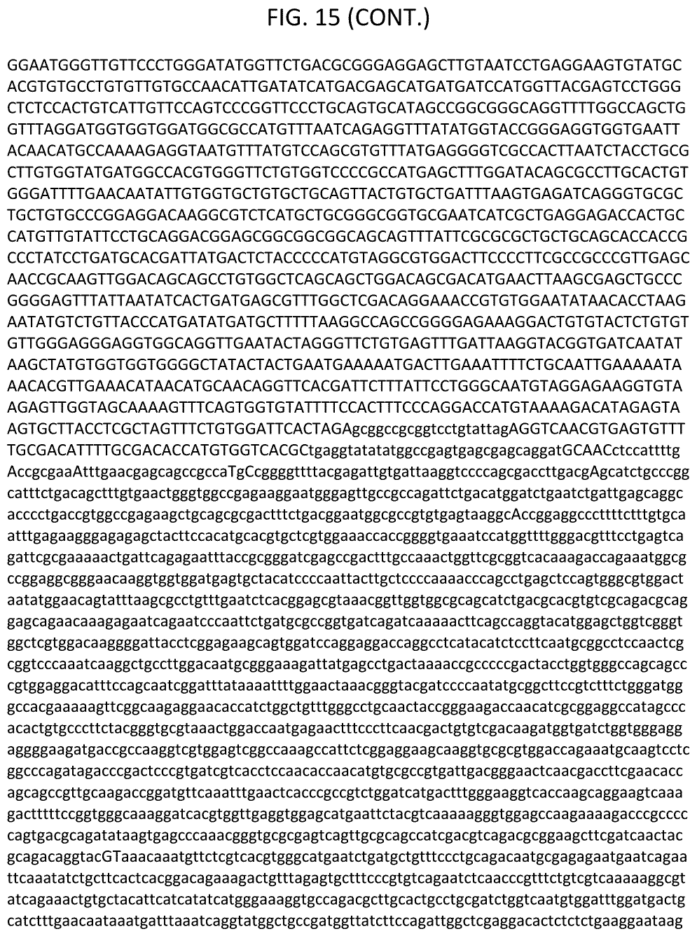

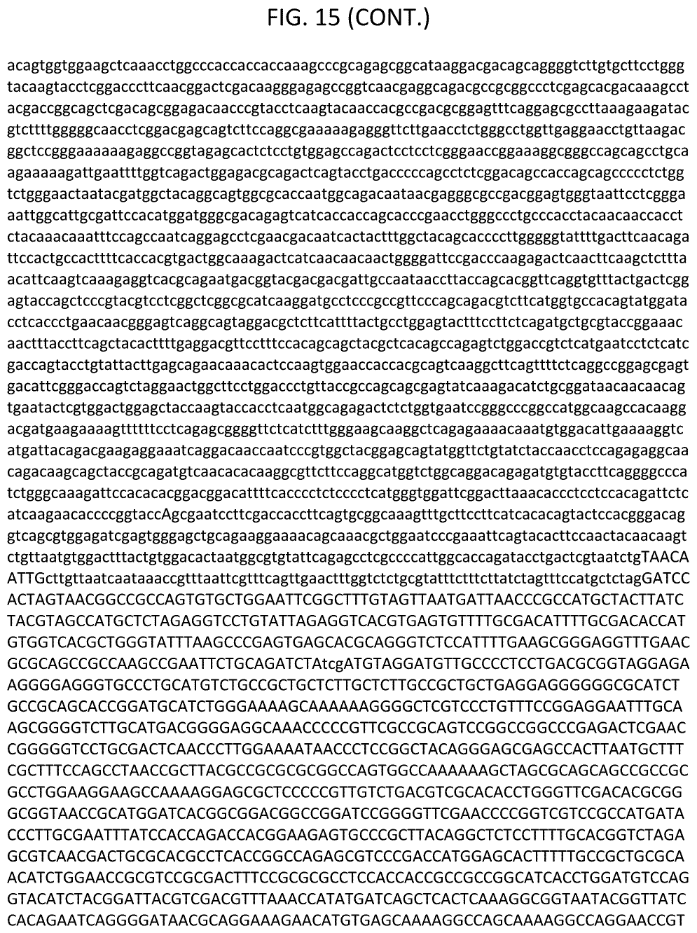

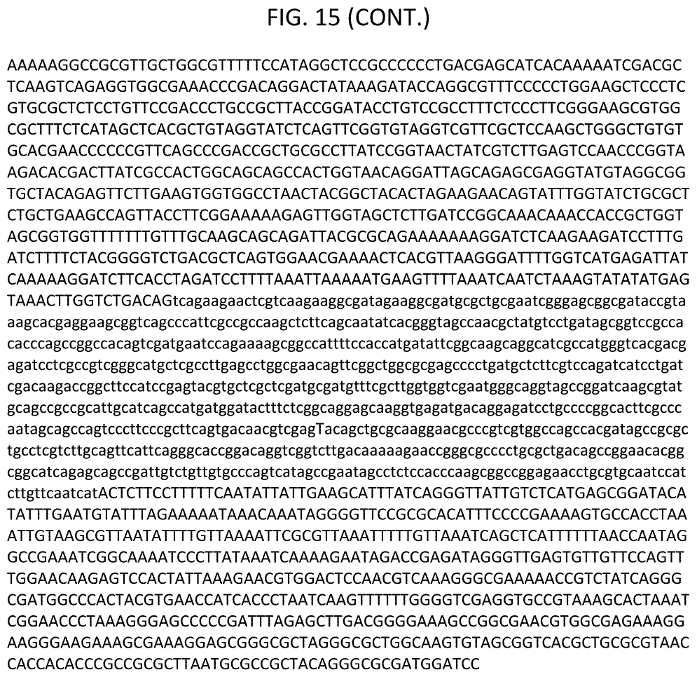

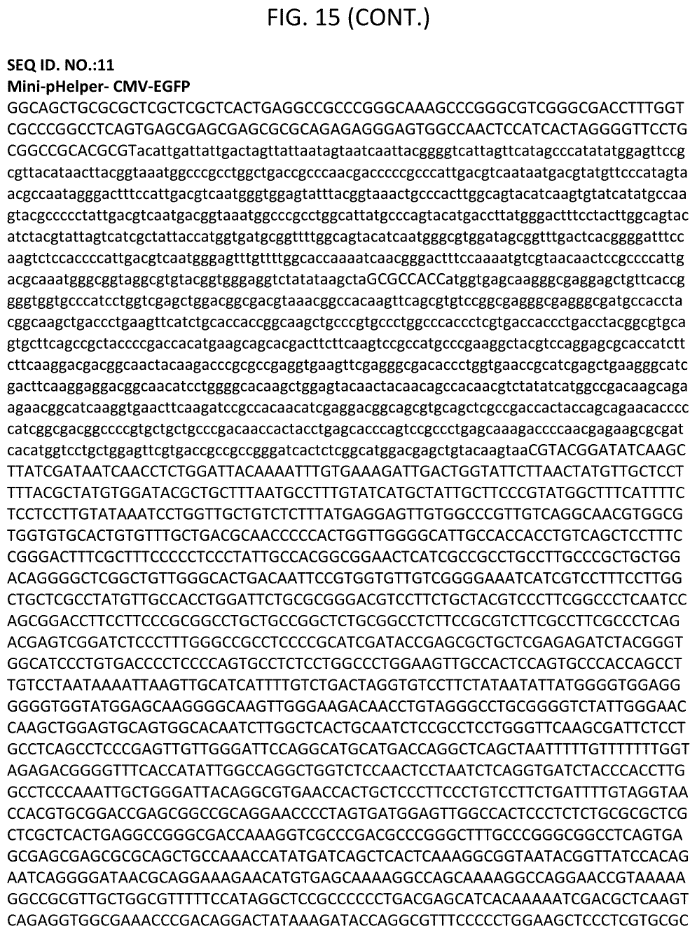

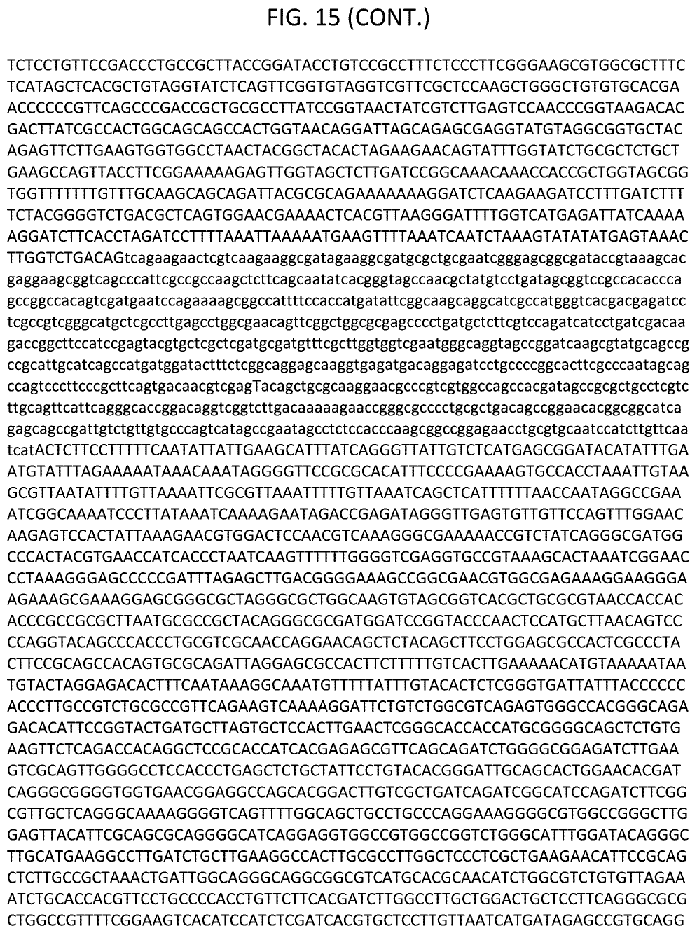

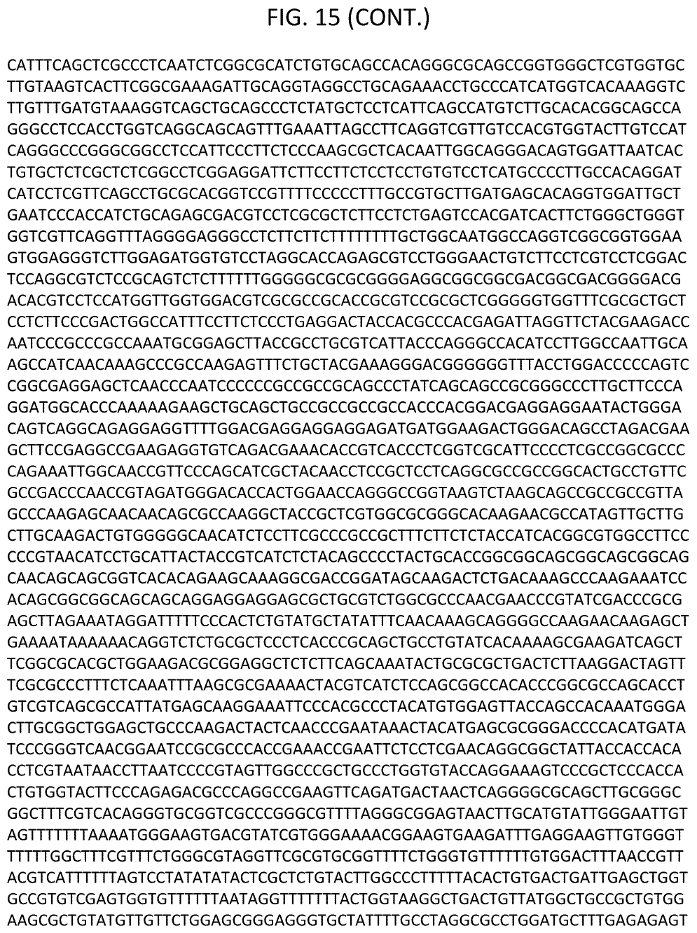

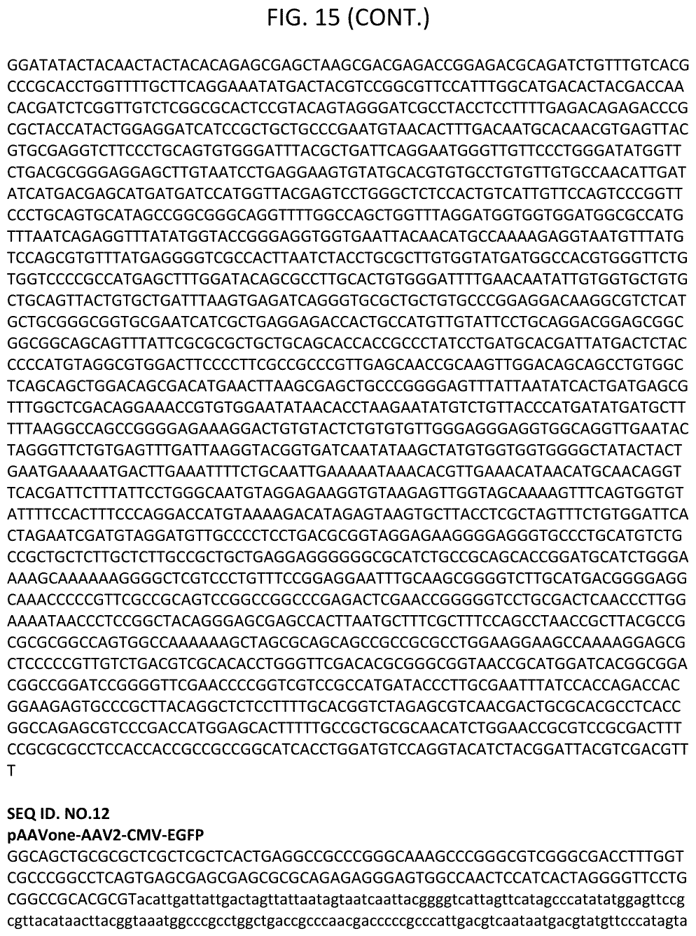

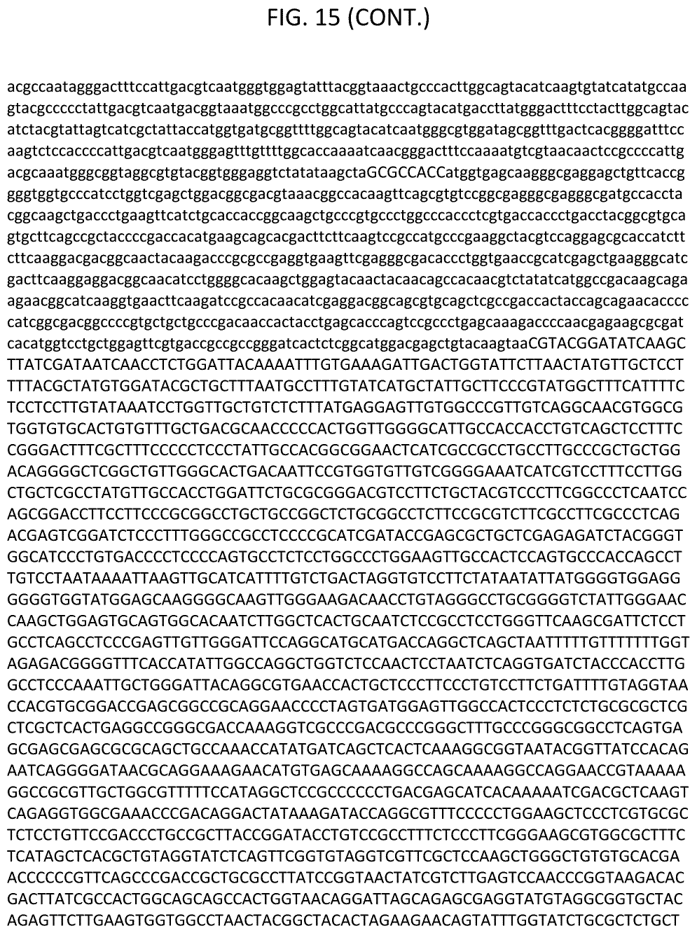

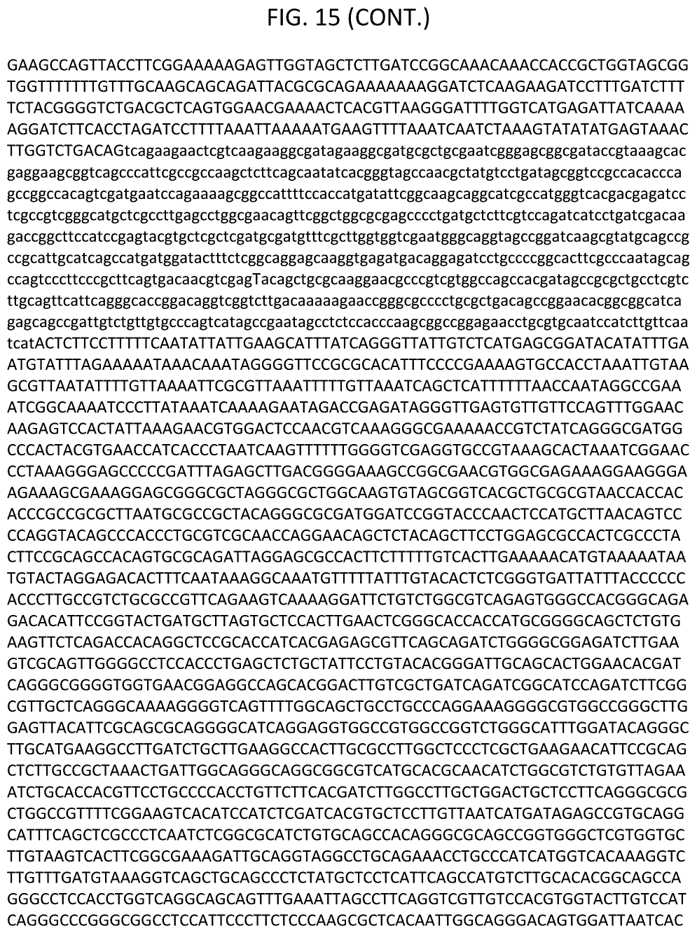

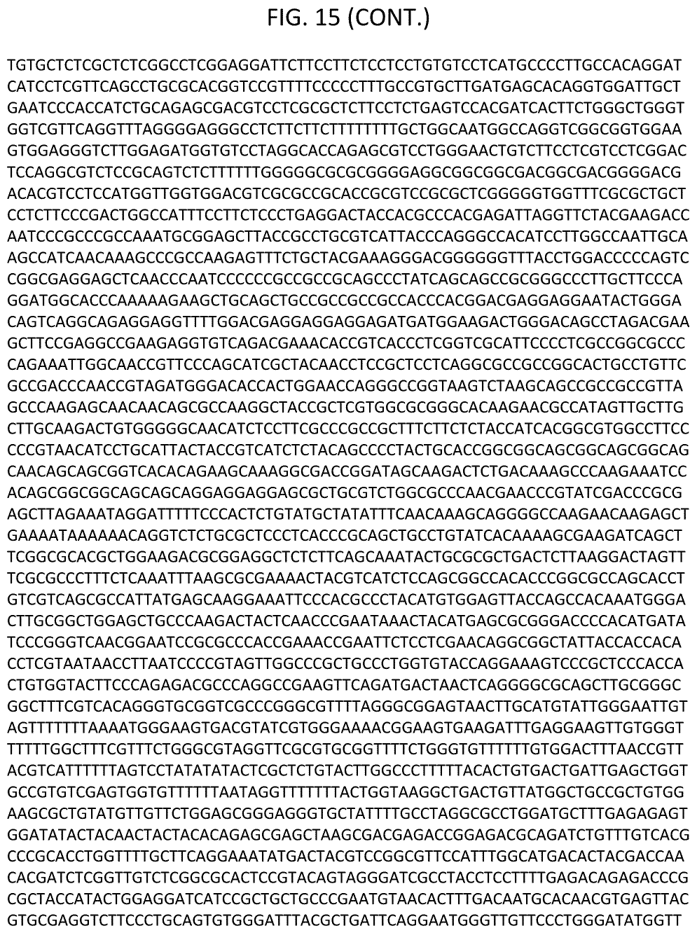

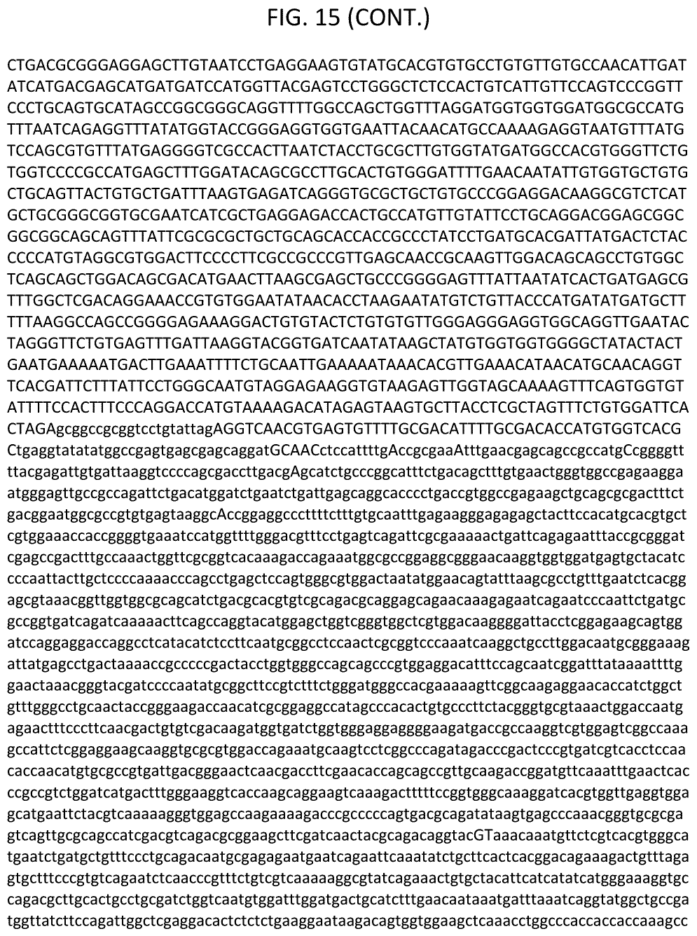

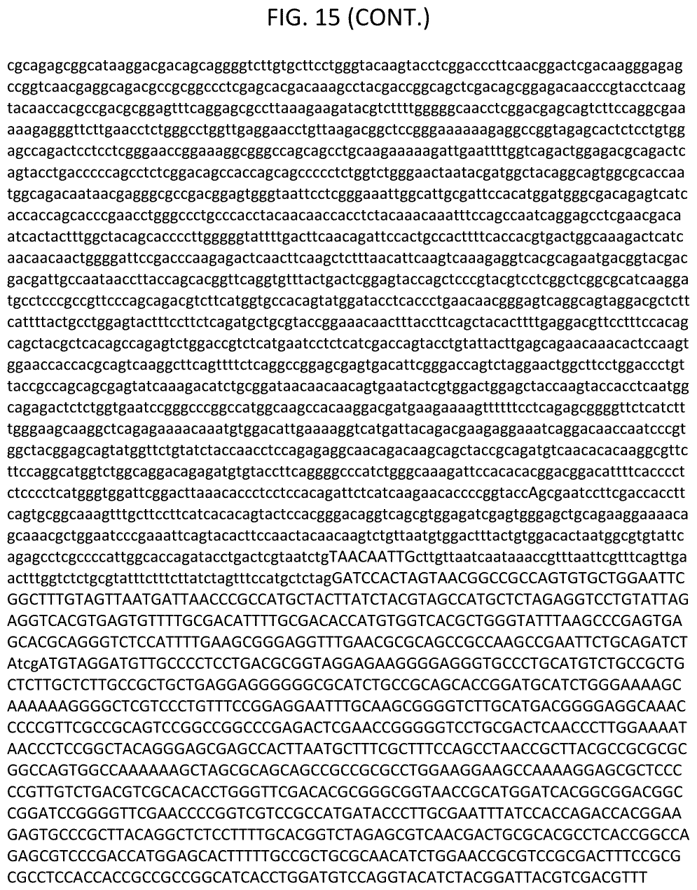

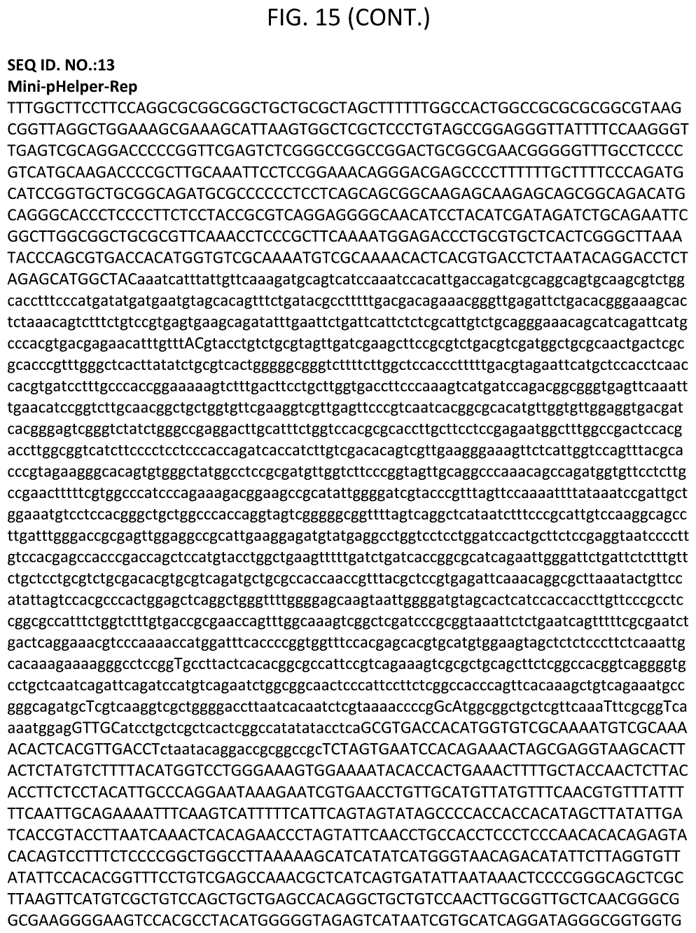

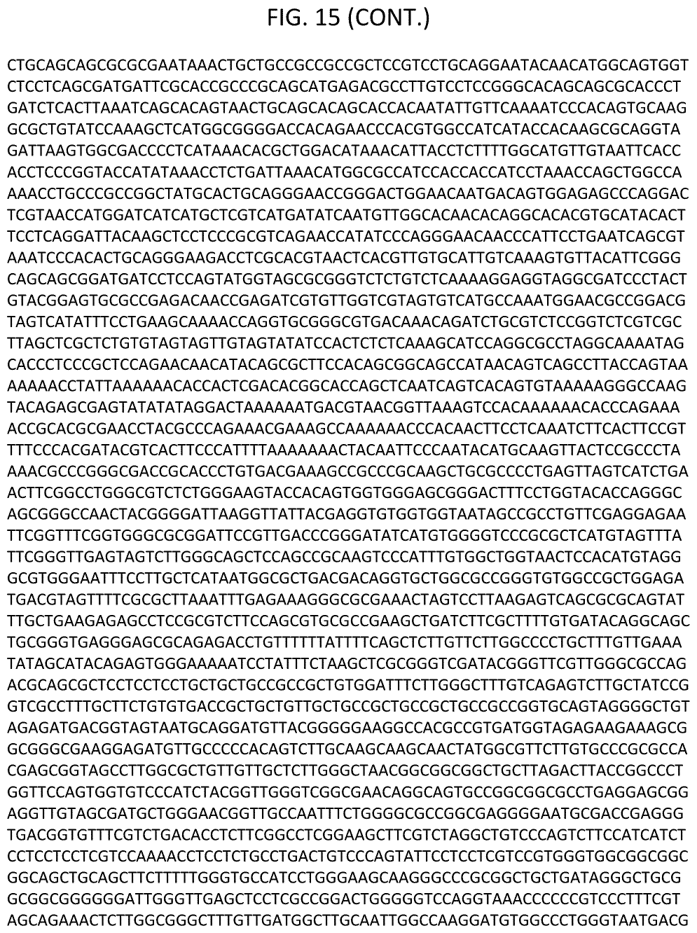

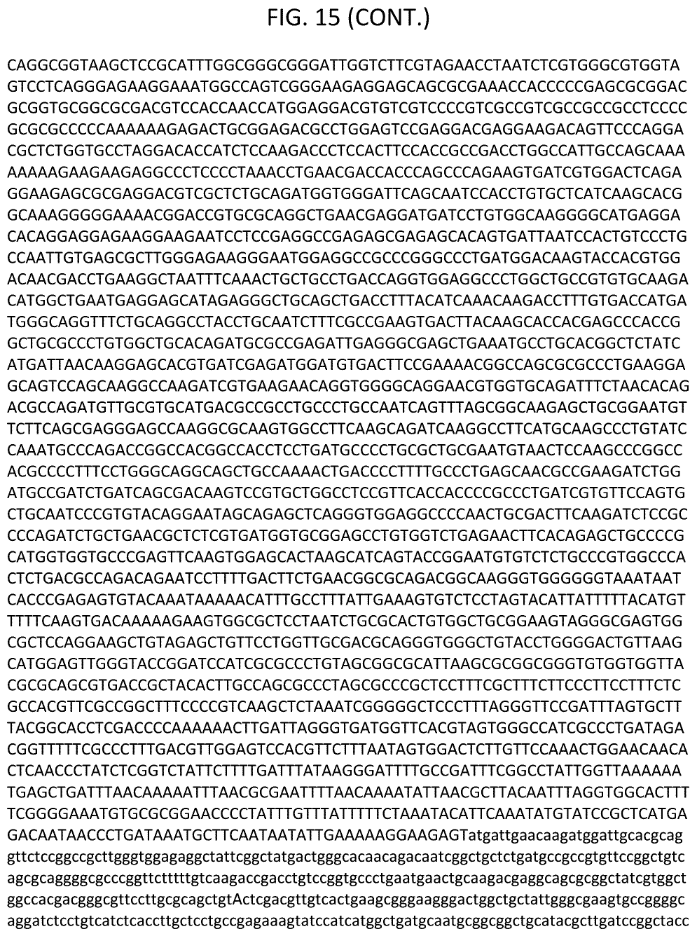

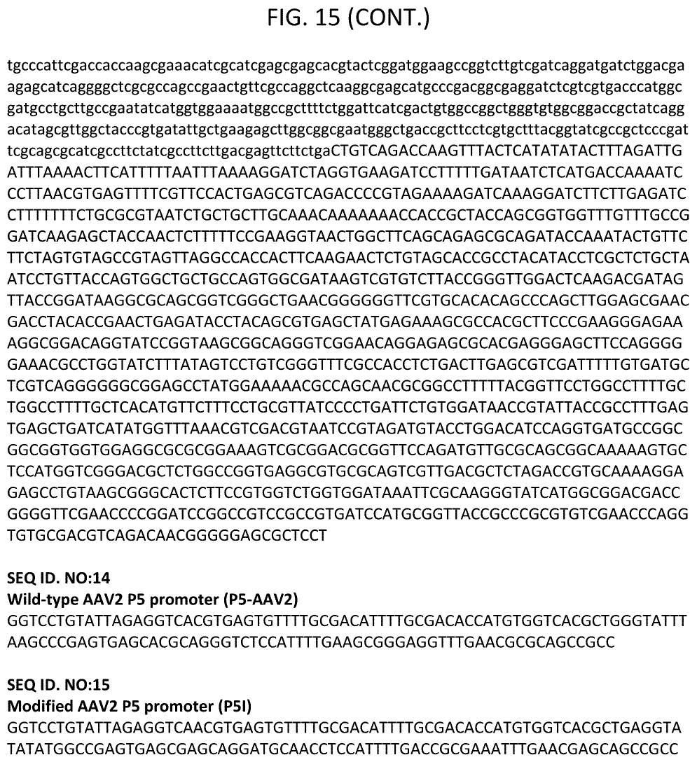

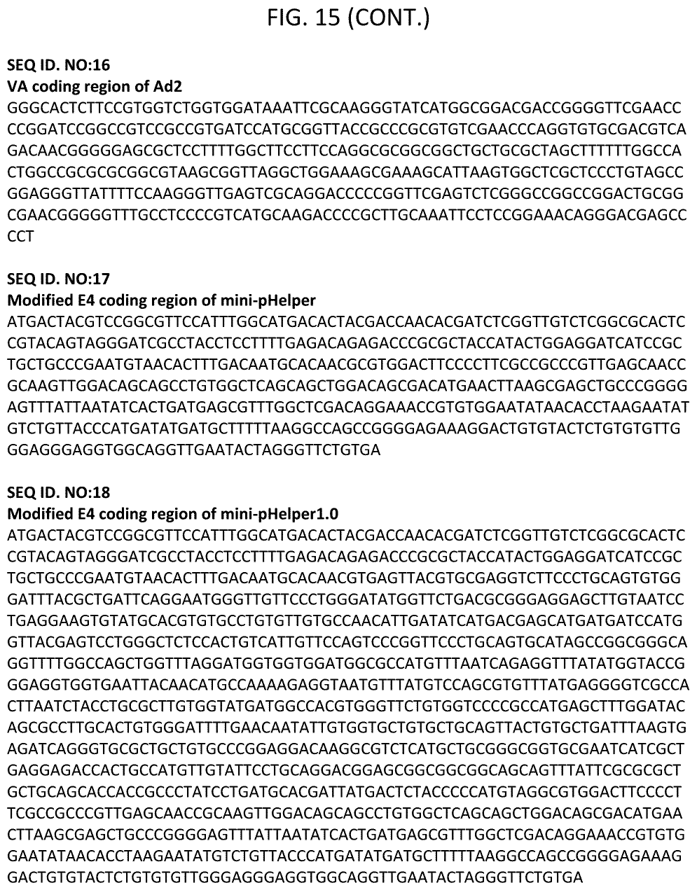

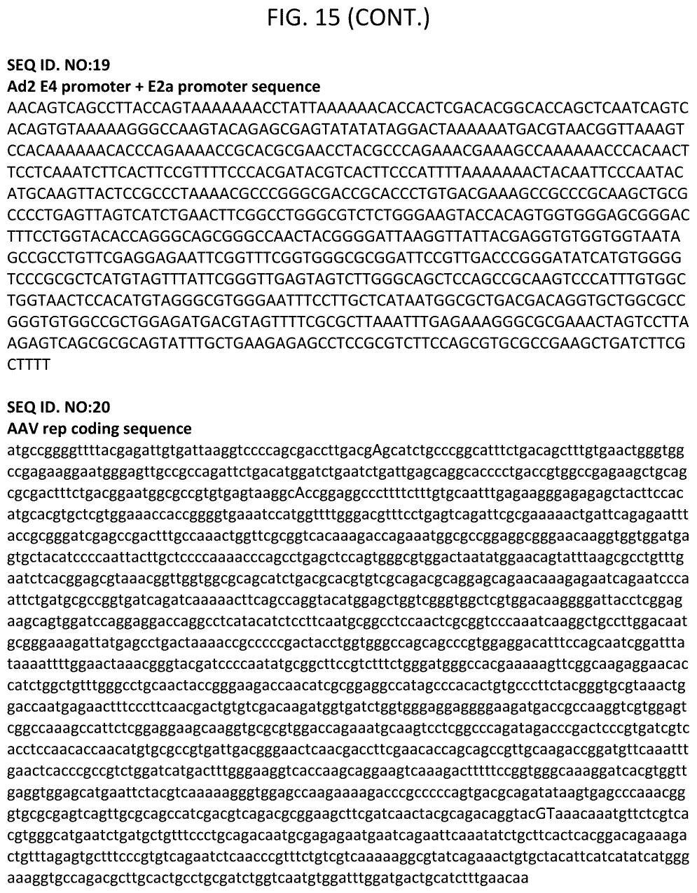

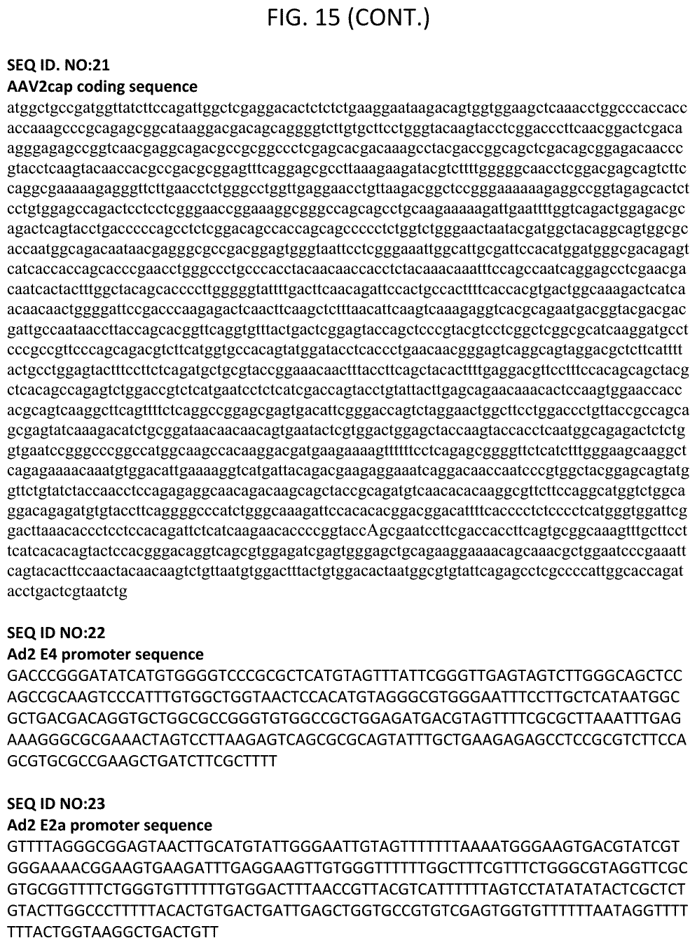

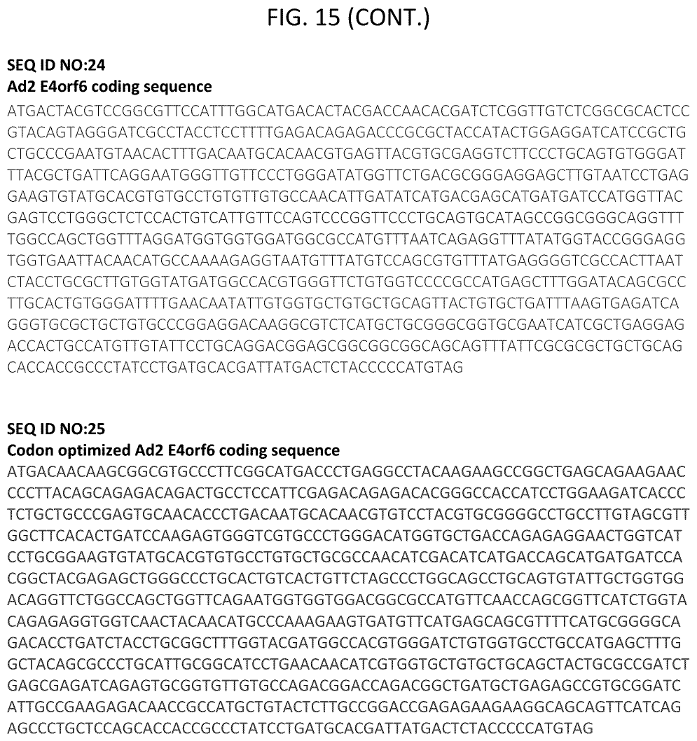

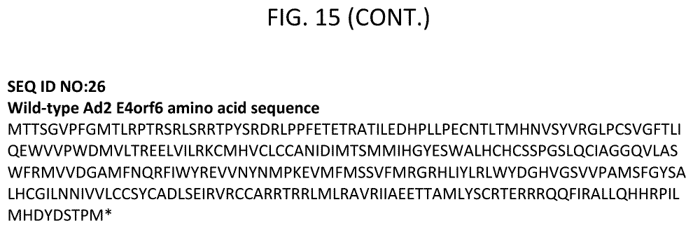

FIG. 15 is a list of sequences described in the present application.

Herein, incorporated by reference is the sequence listing xml file filed on Jul. 6, 2023, called 2037-005 CIP.xml, created on Jul. 3, 2023, and 115,446 bytes in size.

The above description is for the purpose of teaching a person of ordinary skill in the art how to practice the present application. It is not intended to detail all those obvious modifications and variations of it which will become apparent to the skilled worker upon reading the description. It is intended, however, that all such obvious modifications and variations be included within the scope of the present application, which is defined by the following claims. Any patents or patent publications described herein are expressly incorporated by reference in their entirety. The claims are intended to cover the claimed components and steps in any sequence effective to meet the objectives there intended, unless the context specifically indicates the contrary.

What is claimed is:

1. A polynucleotide expression construct for production of a recombinant parvovirus, comprising:
(1) a modified adenovirus E4 coding region that encodes E4 open reading frame 6 (E4orf6) and E4 open reading frame 6/7 (E4orf6/7);
(2) a modified adenovirus E2a coding region that encodes an E2a protein;
(3) an adenovirus VA RNA coding region that encodes adenovirus VAI RNA and VAII;
(4) a parvovirus protein coding region that encodes parvovirus proteins necessary for the production of the recombinant parvovirus;
(5) a recombinant parvovirus sequence comprising (1) an expression cassette comprising a nucleotide sequence encoding a transgene and a regulatory element operably linked to the nucleotide sequence, and (2) at least one ITR at one end of the expression cassette; and
(6) one or more regulatory elements that allow expression of the E4orf6 and E4orf6/7, the E2a protein, the adenovirus VA RNA, and the parvovirus proteins necessary for the production of the recombinant parvovirus in a host cell.

2. The expression construct of claim 1, wherein the modified adenovirus E4 coding region comprises (i) partial or complete deletion of E4orf6/7 intron 1.

3. The expression construct of claim 2, wherein the modified adenovirus E4 coding region comprises a sequence corresponding to nucleotide sequence 32645-35835 of the Ad2 genome with a partial or complete deletion E4orf6/7 intron 1, wherein the E4orf6/7 intron 1 has the sequence of SEQ ID NO:1.

4. The expression construct of claim 1, wherein the modified adenovirus E2a coding region comprises (i) a partial or complete deletion of early-E2a or later-E2a intron 1, (ii) a partial or complete deletion of E2a intron 2, or both (i) and (ii).

5. The expression construct of claim 1, wherein the modified adenovirus E2a coding region comprises a sequence corresponding to nucleotide sequence 22233-27575 of the Ad2 genome with (i) a partial or complete deletion of early-E2a or later-E2a intron 1, (ii) a partial or complete deletion of E2a intron 2, or both (i) and (ii).

6. The expression construct of claim 1, wherein the recombinant parvovirus is rAAV and wherein the parvovirus protein coding region comprises an AAV Rep coding region encoding a AAV Rep protein and an AAV Cap coding region encoding an AAC Cap protein.

7. The expression construct of claim 6, wherein the AAV Rep coding region comprises an AAV Rep coding sequence operably linked to an AAV P5I promoter.

8. The expression construct of claim 1, comprising:
(1) SEQ ID NO:18;
(2) SEQ ID NO:4;
(3) SEQ ID NO:16;
(4) SEQ ID NO:19;
(5) SEQ ID NO:20; and
(6) SEQ ID NO:21.

9. The expression construct of claim 1, having a size of less than 20 kilobases.

10. A method of producing recombinant parvovirus, comprising the steps of:
introducing the expression construct of claim 1 into a host cell;
incubating the host cell harboring the expression construct for a desired period of time to produce recombinant parvovirus particles; and
harvesting the recombinant parvovirus particles after the incubation period.

11. A polynucleotide expression construct for production of recombinant parvovirus, comprising:
(a) a modified adenovirus E4 coding region encoding adenovirus E4orf6 and E4orf6/7, wherein the modified adenovirus E4 coding region comprises a partial or complete deletion of E4orf6/7 intron 1,
(b) a modified adenovirus E2a coding region encoding an adenovirus E2a protein, wherein the modified adenovirus E2a coding region comprises at least one deletion in early E2a intron 1, late E2a intron 1 or E2a intron 2;
(c) a sequence encoding one or more adenovirus VA RNAs; and
(d) one or more regulatory elements operably linked to (a), (b) and (c).

12. The expression construct of claim 11, wherein the modified adenovirus E4 coding region comprises a sequence corresponding to nucleotide sequence 32645-35835 of the Ad2 genome with a partial or complete deletion of E4orf6/7 intron 1, wherein the E4orf6/7 intron 1 has the sequence of SEQ ID NO:1.

13. The expression construct of claim 11, wherein the modified adenovirus E2a coding region comprises a sequence corresponding to nucleotide sequence 22233-27575 of the Ad2 genome with a 1897 bp deletion of SEQ ID NO:3 that encompasses the complete sequence of later-E2a intron 1 and E2a intron 2.

14. The expression construct of claim 11, comprising (1) SEQ ID NO:18; (2) SEQ ID NO:4; (3) SEQ ID NO:16; and (4) SEQ ID NO:19.

15. The expression construct of claim 11, wherein the expression construct further comprises an AAV Rep coding region and an AAV Cap coding region.

16. The expression construct of claim 15, comprising SEQ ID NO:20 and SEQ ID NO:21.

17. The expression construct of claim 11, wherein the expression construct further comprises a recombinant AAV genome comprising:
an expression cassette comprising a sequence encoding a gene of interest and a regulatory element operably linked to the sequence encoding a gene of interest; and
two AAV ITRs flanking the expression cassette.

18. A method for producing recombinant AAV, comprising the step of:
introducing the expression construct of claim 11, an AAV trans plasmid encoding an AAV Rep protein and an AAV Cap protein, and an AAV cis plasmid encoding a recombinant AAV genome into a host cell;
incubating the host cell harboring the expression construct of claim 11, the AAV trans plasmid and the AAV cis plasmid for a desired period of time to produce recombinant AAV particles; and
harvesting the recombinant AAV particles after the incubation period.

19. A method for producing recombinant AAV, comprising the step of:
introducing the expression construct of claim 15 and an AAV cis plasmid encoding a recombinant AAV genome into a host cell;
incubating the host cell harboring the expression construct and the AAV cis plasmid for a desired period of time to produce recombinant AAV particles; and
harvesting the recombinant AAV particles after the incubation period.

20. A method for producing recombinant AAV, comprising the step of:
introducing the expression construct of claim 17 and an AAV trans plasmid encoding an AAV Rep protein and an AAV Cap protein into a host cell;
incubating the host cell harboring the expression construct and the AAV trans plasmid for a desired period of time to produce recombinant AAV particles; and
harvesting the recombinant AAV particles after the incubation period.
